SEQUENCE LISTING (98 sequences)

SEQ ID NO: 1 - DNA, 22743 bp, Homo sapiens
ggagttagcgacagggagggatgcgcgcctgggtgtagttgtgggggaggaagtggctagctcagggcttcaggggacagacagggagagatgactgagttagatgagacgagggggcgggctgggggtgcgagaaggaagcttggcaaggagactaggtctagggggaccacagtggggcaggctgcatggaaaatatccgcagggtcccccaggcagaacagccacgctccaggccaggctgtccctactgcctggtggagggggaacttgacctctgggagggcgccgctcttgcatagctgagcgagcccgggtgcgctggtctgtgtggaaggaggaaggcagggagaggtagaaggggtggaggagtcaggaggaataggccgcagcagccctggaaatgatcaggaaggcaggcagtgggtgcagggctgcaggagggccgggagggctaatcttcaacttgtccatgccagcagcccctttttttccagaccaagggctgtgaacccgcctggggatgaggcctggtcttgtggaactgaacttagctcgacggggctgaccgctctggcccagggtggtatgtaattttcgctcggcctgggacggggcccaggccgggcccagcctggtggagcgtccaggtctgggtgcgaagccaggcccctgggcggaggtgaggggtggtctgaggagtgatgtggagttaaggcgccatcctcaccggtgactggtgcggcacctagcatgtttgacaggcggggactgcgaggcacgctgctcgggtgttggggacaacattgaccaacgctttattttccaggtggcagtgctccttttggacttttctctaggtttggcgctaaactcttcttgtgagctcactccaccccttcttcctccctttaacttatccattcacttaaaacattacctggtcatctggtaagcccgggacagtaagccgagtggctgttggagtcggtattgttggtaatggtggaggaagagaggccttcccgctgaggctggggtggggcggatcggtgttgcttgcctgcagagagggtggggagtgaatgtgcacccttgggtgggcctgcagccatccagctgaaagttacaaaaatgcttcatggaccgtggtttgttactatagtgttcctcatggcgagcagatggaaccgggagacatggagtccctggccagtgtgagtcctagcattgcaggaggggagaccctggaggagagagcccgcctcaattgatgcctgcagattgaatttccagaggcttaggaggaggaagttctccaatgttctgtttccaggccttgctcaggaagccctgtattcaggaggctaccatttaaagtttgcagatgagcttatggggggcaatcttaaaaagtccacagcagatgcatccggctcgaggggccatcagctttgaataaatgcttgttccagagcccatgaatgccagcaggcacccctcctttcctggggtaaaggttttcagatgctgcatcttctaaattgagcctccggtcatactagttttgtgcttggaaccttgcttcaagaagatccctaagctgtagaacattttaacgttgatgccacaacgcagattgatgccttgtagatggagcttgcagatggagccccgtgacctctcacctacccacctgtttgcctgccttcttgtgcgtttctcggagaagttcttagcctgatgaaataacttggggcgttgaagagctgtttaattttaaatgccttagactggggatatattagaggaagcagattgtcaaattaagggtgtcattgtgttgtgctaaacgctgggagggtacaagttggtcattcctaaatctgtgtgtgagaaatggcaggtctagtttgggcattgtgattgcattgcagattactaggagaagggaatggtgggtacaccggtagtgctcttttgttcttgcttcgtttttttaaacttgaactttacttcgttagatttcataatactttcttggcattctagtaagaggaccctgaggtgggagttgtgggggacggggagaaggggacagcttggcaccggtcccgtgggcgttgcagtgtgggggatgggggtatgcagcttggcactggtactgggagggatgagggtgaagaaggggagagggttggttagagatacagtgtgggtggtgggggtggtaggaaatgcaggttgaagggaattctctggggctttggggaatttagtgcgtgggtgagccaagaaaatactaattaataatagtaagttgttagtgttggttaagttgttgcttggaagtgagaagttgcttagaaactttccaaagtgcttagaactttaagtgcaaacagacaaactaacaaacaaaaattgttttgctttgctacaaggtggggaagactgaagaagtgttaactgaaaacaggtgacacagagtcaccagttttccgagaaccaaagggaggggtgtgtgatgccatctcacaggcaggggaaatgtctttaccagcttcctcctggtggccaagacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttcaaattgtagtctttgtaacaaccaaataaccttttgtggtcactgtaaaattaatatttggtagacagaatccatgtacctttgctaaggttagaatgaataatttattgtatttttaatttgaatgtttgtgctttttaaatgagccaagactagaggggaaactatcacctaaaatcagtttggaaaacaagacctaaaaagggaaggggatggggattgtggggagagagtgggcgaggtgcctttactacatgtgtgatctgaaaaccctgcttggttctgagctgcgtctattgaattggtaaagtaataccaatggctttttatcatttccttcttccctttaagtttcacttgaaattttaaaaatcatggttatttttatcgttgggatctttctgtcttctgggttccattttttaaatgtttaaaaatatgttgacatggtagttcagttcttaaccaatgacttggggatgatgcaaacaattactgtcgttgggatttagagtgtattagtcacgcatgtatggggaagtagtctcgggtatgctgttgtgaaattgaaactgtaaaagtagatggttgaaagtactggtatgttgctctgtatggtaagaactaattctgttacgtcatgtacataattactaatcacttttcttcccctttacagcacaaataaagtttgagttctaaactcattagaattgttgtattgctatgttacatttctcgacccctatcacattgccttcataacgactttggatgtatcttcatattgtagatttaggtctagatttgctagctccaagtaattaaggccatgtaggagagcatggtaaccacagatagaactggtattatcccaagtggtctgcagactgctgagtggggatgggatctgctctctgttgagagttggtaatcattggtttgaaatgtgatgaaaccactcaagccaatgaaggtgggtgtgtaggtggggagtactttgccataatattttaaaacattacctggttagagttctaagtggtacttatttttgtttggttaggggaaagcctgaataaaaacagaaatggacacataatatgcatattccatagtctttgggaggctggaatgtgcctgggatttgggtctaagtgtatgcgtaattcttacctcactaaagaatttgccttgtttttttccttttggtgagtgactaaaacgtctgggcttccctgtgtgcgtgctacagtaagcaagcagaggctgtgcaaaggtgtgagcaggatcacgtggaatctggaggatacatcttggcttgcaaactgcctctgtctcctgggtgggactgttctgtccttgcactgctgttctgtgttacctcttggggtgtaaggttttgcttacaggagacaaactttgggcgtagaatggaagccactgccagcctctgtgctgagaaggaaggtgcttgtttcaaagggagcagcaagggaggcttgttctactcacctgggcctgtttgcctgagaaggggagataagggctgaactgggactagccagggggaccaacacaaatggtgggggatcatgacctgaaggattctttccttcccatgagctgcagggctggttgccgtccttgcaactgtgtcttatttgcctgtgccgttatatcttggtgacccctccacgtgtacactactgacaaacgggtggagtgctggggagaagtcactgtgccgcccacctagtaaaccttctgtctgtgctcatggcatctccaagatggggcactgctgtgtgcagaatccagggtcctctttctgcttgcaactcctttccctggatgccccagaaacaatccaggcctcctttcctatcttacccctttgctttgctttttaccccagcacctctataaccgccttctcttcttttcagaactccttgtttctcgtcctgttttttatgattacaaaactcttgcttccaccctggaagataactgctatagatgcctgtatgtaaatggtgctgtctccagcaactggcatgctgaagaagaattgattcacggggtataaatgttggggattggaagtggggatgaaatggcacttgttgatacaggagcagagaggtgaggccgactgctgaagacagctcgccaccctccttgcctccactccaatccaggggctggggccacattctttgccttcatttatcctcagatcaggtgagatcgacaggaggtgttgatggcagtgccagcaattattgctaatccgtttgcatccttatgcatagatctgaattcagactttgtgaatttccagaggtgtgggtaatataatagaattcagtgagtgggcatggctgatcttgtgcaaattaaaagttatggggcataagaatagcaaaagttgaacttcttttaaaaaggaaagtaccctgagagccagtattggttgaggctcttcagtatgcccaggttggcagcactgagaaccgcaggaacggcctgttgttacaaaaaggagattgactcagctgcccttggtgcatctgactgactatgactgctgagagattccaaggacccttaatgccagggctaacctctccatgtgcagtgagacctctggaggaagtgtcatcctctggctttgtgtggtactcattatggtgcagtgcgggcatgaaatgaagacacccaaataggcttacagatacgatatgttttaaatgttcgtatttaacaaaaacatactgacactgtttggaaatggcaacaggaagatagcaaaatgaatactaacattacgaaaagatgaacaggtacatgttccaaggcaggtggctgtgaacttcctctgagtgaaggcatcccctccagcacctttcagcctgctagttaggacgacccgccgccaccctccaggacctccagccctgcactgcctttcctctcttttaaataattcttcattgagttctaatatgtaaaaaaaaaaagtttactgtaaagtttgcaaataaggaaattttttttaaaagtcctcagtaatcttaccagtaacaattgttatgggcacatttgcttttggaagatttcttttgtatgcatgggataagtacatttttaaacaaaaatgggattatgccataaattctattttgtgactttaatatatagtgaacaccttttttaatgatgacaggatgttcccttgcatggctgtatcaatttaaacaatcttgtttcaatgggcatacagggtattttctagtttttttttcctcttagaaaataatacttgcgatgactttccttgtagctcagactttttcacgtctgttgttatctctttgggaatgctgaatacatacatttcgagaaggaaatgactgttaaactcttaagacttcaggttcatattgctaaactgcccagcagggagggattttttcaattagtgttctcactggtgaggcaaacctgatgccttcccctcttcctcagaaccggctttatcacattgaaaacctttgctcctccgacggatcgagtctgctttccctctggatgtgagcattgctttgtctgctggtgactgaacatctctaccttgtgtcaattggccatttgtggtgtgtgtgtgtgtgcgtgtgtgtgtgtgtgtgtgtgtatgattttctaattcctagtcatttttctattgattgttttgcaaaagccatttacatcttaaggatattgataatcttttgttatatttgatgcaaatatttttttccagtttataggttgccttttaattttgtgtttcaggtagataaaagttaaacgattttcttaggttagtttatcactgtggtttctgaacttgttatgtgtagatcttttccaccccaagagtacataaatattaatccatactttcttatggaacttgtatggtttcgttttttacatttaaaccttcttccccgtggtgtgtgttgtggaatctgtgtttgtgtgaggaggggcatggtgctctcagaacccacctcctgtggccagagagccctgtcctgtgagggtggttgtcacagtggcagggttcaattcagaagaccttgagggcaggctgatgtttcctgaatgggcccctggttgttgcttgtccctgactctccatttccccatctgagtggatttggacctaatagggcactggagctggttcgaatcctgactggactacttggcaactttatgtctgggagcaagttacttaacctccccaagcctgtgtctgtgaaatgcgggtaaatgaatgtagatgtttggcagcagctactccttgttgagctctcacagtgaactctcctgcctctgccctccttccccgcctcccctggtgcctagcgtcaggtctagccacttcctcctgggcccctctcccttttctgtggctggctgcctgcccgcctggcgctggacctttcatgtaacgggaatcagcatgtatattctggtctggtctgtttctacacttaattttgtttccagtagtatttccctgtaccggcagagttcacaaacacatttgaagaggctttttctcaggattcttaaccttcccaaaggaagtcccatggatgggtttctagaagtctataaatgctctgaaattgtatttttctgtggaaagcataactttcatctgcttgttcgtgctcaaaaaagatcatgaatgaatgattgcatgattttatgccattgtgcttatactaaaggatatgtagcccatctcttgagctgttaaactgttttgactactttaaatcgtgcagctgtgagcatctctgtaaatttagtgtacacatgtatcccctggagtggcattgcctcggcagtgagcacttatggttttataactctcttcacagactcaaatgactccagaaagctacacttcctgttgtgagtatatgatatccatttccctacatagccactaacatcaggtttttacaattttatttatttcttgctactttaagaaatttttgtggtgaaatacatataatagaagttgactatctgaatcatttttaagtatacattcagtagtgttaagtatgtcgccattgttgtacaaccaatctccagaactttttcatcttgcaaaacaaactctgtacccattaaataacattaaacattccattccctccagcctcagcaaccccattctactttctgtttctgtgagtttgactattccaagcacttcatatcagttaaatcatgaagtatttgtctgtctgtgactggcttatttctctgagcacagtgtcctcgagatgcgtctatgttgtagcatatgtcagaatttccttcctttttaaaagatccaaataatattcttattttatatcttttttttatccattcatccattagtggacacttgggttgcttttggctattgtaaataatggtgctatgtacaaatatctatattattgtatttacaagtataatgctgtaatgtacacacatctttttgagatcctaccttcagttcttttgagtatatagccagaagtggtattactaaatcttacgatatttctatttttaatttattgaggaaccactgtagtttttcatagcaactgcaccattttacgttctcaccaagagtgcacaagggttccgaggttcccacatcctccccaacacttgttattttctgctttttttagattgcagccatcatagtgggtgtgaggtgacatttcattgtggttttgatttgcatttccctaatgaggagtgatgctgagcatcttttcatatgcttactggtcatttgtatgttgtctttggaaaaatgtctattcaagtcctttgactattttaaaaattgggttattagagttatcgttgttgttgacttgtaggagtttctttctatattctggatattaatcccctatcagatatatgatttgcaaatatcttctcttattccataaggttactttttcactttgttgattgtgttctttgatgtatagaagtttttagttttgaaatagtctaatttatctgtttttacttttgtggtctgtgcttttggtgtcatatccaagaaatccttgccaaatccaacgttataaggtacttttaaggtattttagttgtcttagtctatatttctgtactcacctttctttatccactcatcagttgatgggcatgtaggttggttccatatctttgcaattctgaattgtgctatgatcaggtgtctttttagtataatgatttactctcctttgggtagatacccagtagtgggattgctggatcgaatggtttttataattttctattttaccacagtttctctctgcatttttcctctttgaccactaaccatgtgaaattctcatattgacctttataatgatcatgaactcttagtatcattgggaaggccacatttgccacttatgattgtaaaccttatcctccatttttcctgttattgttggtgcaaaaagcacctattataccaggactttaaaaatcagtctgataagtctttgataagtctaataataataactgataagtccattgaatttgcttctgattactttttctttagtagctaaacatgtatgtactcctatgattacaatgaacactcctctccatttaaattaattatttacattgatgaaatagcaaaatgttaatgactaaatactgtcttggttttttcgttccaggtcagtcaatattaacttcttataattttcttttttttctttatgtgtgtgtgtgtgtgtatttttttttttttaatttcaatggcttttggggtacaaatggcttttggtcatatagatgaattctacagtagtgaagtctgagattttactgcaccggtcacctgagtagtgtacattgtacccaatatgtggttttttataccttgcccccctcttaccctccccactttgagtctctagtgtccattatgtcactctgtatacctttttgtacccataagttagctctcacttataagtgagaacacacagtatttggttttccattcctgagttgcttcacttagaataatatcctccagctccatccaaaattgctgcaaaaaaaaaaaaaaccacaaacattattttgttcttttttattgctaagtcatattccatggtgtagagataccacattttatttatccactcactggttgatgggttggttccacatctttgcaattgtgacttgtactgccatcaagtgtctttctggtataatgacttcttttcctttgggtagatacccaggagtgggattgctagatcaaatggttcttaacattttctctctggatctatttctggaaattttaggctccagtttttgttgttgttgttaataaaatgcaatggaatgtaatgatcatcacttttcattatgctttaaaatctggtaaatggaggctagaacactcctgtaaggcaagaatattctctctgttggaactcaaatacacagaactgggtaaatctcaatcttaatctttgattcaggacacaacatggctctcttttacttgctttctttaattgttttttaataatgtggtaagcatttctgaatctcctatccaatacaaaaactaggacaatacagacagtaactcctatggttacaatgaacactcctctccacttaaattaattatttacactgatgaaattgaaatagcaaaattttaatgactaaatactgtctttgattttttgttccaggtctgtcaatattaacttcttataattttctttttttttctttatgtgtgtgtgtgtgtgtgtatatatatatatttaatttcaatggcttttggggtacaaatggcttttggtcatatatatgagttctacagtagtgaagtctgagattttactacaccttccacttatgtggtcccacaccacccgcctcccctgccgcctcctgccaccccctaggccaaggtaataatcatcctgaatcctgggtttatctctcacttgctttcttttcatataattttgcaaaagaatctgatctaaatgtgtttttcagagtatatatttatattttagctgttcttagagaaaatttattattttgcatgtaatcttatggaacattctcatttaataccatggtaagattcagcccttgcccaggggatagttcatttagtttgtttactggatagagctcatcatgtgactatacctcagttagtttatcagttctcccatccatggtgactaggttgcctctcagcctctcaacaacactgtttctcagtgtccttgtagaagtgatatgtgggtgttttctccttacacagagttgaaaggtgacgacaacaacgttggcactaccaatcccccaccctccagaggggtaaccagtgttaccagtttgctgtgtttcctgctacacctcgccttattcacttccatttgtatctgaaaaacgtgttgcatggtttcttttctatagaagtggtaaaatgctattgtgtcctgtacattattgattactttttttcatttaacagtagggagatgcctgggagtacacagagaactgccctcattgttttcaacttctgcactgtatgtctgtgagtttagccattctgctgttaatggaaatttacagtattctaatcttttgatattacaaacagttctgtgcgatcatcgtcatacacaaccccttgtgcacaatgcatgagtgtttctcagggtaggtaccaagaagtgaaattcctgggtcatagggcgtgagtccgacatttttctccattctgccctgttgccctccagagtgggtgtccagctttgcatacctaagtatgagagtatctgttgttcatatcctctacgacgctccatatatgaaacttaagtttctgctagttgccatctttgatctatcatgtatgcagtgacctactaagactgtaattggtacagtagattcttgtcatctgtgtgtgaatttagcattcatgggcttaatgctgacaaggcccccagggtccaagacatataatcatgtataattttgtcaaggtataattttttaaattgcttttgtcatgtgtctgctggtgatgcccaacccagtgctctgcacccaggtcacactgtggctttgtcctctgcttatgcctgcattgcagcaactgtcctgaagagaccaaaattatgcagatttaggtaagtccatggctaatgttattatattatgtgctattgtaatggatggggctgtggagtgtatgaatttataaatcactggtcttgtaattaaaattcaaacactatagaaaaaggccatgtagaagataaaagttcctctataatcccggacccctaagataactactaatgacaacttcatttatattccttcagacattttctggctgtggatgtactaaaatgtatcctattattctctgccctaaaatggaatcatacaaggtgtactgttatttttatggctctataacatgtcatattgtacgtgttggtatggtcattttaaccatttttctagtgatggctttgaggttatttgcagtttcctagccatctcaaagtgtgctgcggggatctcttttgcatccctctgggtgcagagctgaggcacccagaggcagtgtccagaggaggcagcatctgtaggtgtcttcacctgctctggctcttggcacatctggttggtgacactgttttgtgagatgggttgaaagcacgtgctgccaaaatagaataatgttggtcctctcctcatgtgccgtggaactggggtaaaactgcgtagtggctgcagctgcctgtccataccggaatcgagtataacacggtgcctggcttagcacaaaacagtagtgggtcctgcaggccccagagtctaattcctggtattctttcccctacacagattaaataaaccaaaaacaaactattctaggaaagcgtctgtgacatttgtaaaaagtggtatttaatgatcttttattcacttgtctgtttagtttgttgaaatcttaagtggcatcctggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgttaaaaatgccagtaagttcatacaaacccagcttgcacccaaggtcacattcagagagcgtagggctgggatgggttgttttccaagcttctgccactgtgtggctagctcttcccactgggaagttctgtgtacccggaatgtcggagtggagtcctgttctagtgtccagcacctgaccctgtgcccaacccctcaacagcctattcctgctgtccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtctgtgctgagccttggcccactttttaattaggcgtttgtctttttattactgagttgtaagagttctttatatattctggattctagacccttatcagatacatggtttgcaaatattttctcccattctgtgggttgtgttttcactttatcgataatgtccttagacatataataaatttgtattttaaaagtgacttgatttggctgtgcaaggtggctcacgcttgtaatcccagcactttgggagactgaggtgggtggatcatatgaggaggctaggagttcgaggtcagcctggccagcatagcgaaaacttgtctctactaaaaatacaaaaattagtcaggcatggtggtgcacgtctgtaataccagcttctcaggaggctgaggcacgaggatcacttgaacccaggaggaggaggttgcagtgagctgagatcatgccagggcaacagaatgagactttgtttaaaaaaaaaaaaaagtgacttgatttaagggaaaaaatgactggctatattcagtcagatatggcaaaaagtctcaaggtgttaatgtgaatgattaaggtcttggggggggtgtcccctatcagactacaggtgtttagaggcacagaaaaaggtgcagttgggttcttaatgtgaaatgatgagaagcacaactccagtgtgtctctttgtgtagaatgtcagcagacaccccctgctagatgtgctggatcatgggaaagcatttccatttgttactagattgttcagaagttttaatttatgatgggtgtggtggctcatgcctgtagtcccagcactgtgggaggctgaggcaggaggatcatctgaggccaagagttcaagatcagcctgggcaacatagtgataccctatctcttaaaaaagaagaagtttttaaatttgaaataataataggtactggatttatgcaaatgtcttttctgcgtcttttgagatgagtatcaggtttttttttttccttttatcatctgatgatgaacttaatgtttccatttgtattaatggaatactaagtccctctgtgatttctgaaccaagctattcctaggcctgagttttattttgttgacacagaaataaattagaaggccaagcgtggtggcatgtgcctgtagtcctagttgctgaggtaagaggattgcttgagcccaggagttcaaggctgcagcaagctttgattgcgccactgcactccagccttggcgacagactaagacgctgtctcaaaaaaaaacaaaaacgacaaaaaaaaaacaaaacagaaaaaataaactaaggcaatgacagtccctggcaaatgctgggagggaggcagcagtggtcagggaaggtaaccctgaagcaggacttgtaaagcaaataagattgggaggccaaggtgggtggatcacgaggtcaggagttcgagaccagcctggccaacatagtgaaaccccgtctttactaaaaatacaaaaaaattagccaggtgtggtggtgggtgcctgtagtcccagctacttgggaggctgaggcaggagaatctcgaacccaggaggcggaggttacagtcagctgagaccgcaccattgcactccagcctgggtgacagagcaagattccgtctcaaaaaaaaaaaaaaaaaaaaaaccaagaagaaaaggaatgaattagaacttcttctgcttggacttaagggcatcatcaggcaggttttgggtaggatagcaggggaggcagagacatagtcggggtcagtggtcatgagtgtggctttgagcccaaaaacttggtttctgttccctactttgccactcagtagtgcatgactttggccaaatttcttaaattcatgaagcaagtttccgggtgaatgaaatggggataaaaatagtgttcaaacctatccgttggtttgtgtgaaactgaaatgaatagtatcgtgcaggtacttgtgagcaaggggagctgctgtttcctgtccctttatgatgggaaatatctagacaagttcccaaccctctgcactgcaggctgcatggcacggagggtcttgtaacaccagctggggctggccttcttttaggagcttcagtggttctgaaaacttttatttgtttgtttgttttagtagatgtggggtctttctgtgttgcccggactggtctcaaacttctggactcaagtgatcctcccccgctcaacctcccaaagtgttgggattacaggtgtgagccactgtgcccagccttgaaaactttttcaggttcttccagggttactgggctattaaatatttctatttcattataagtcagtttttcaaagttatattatcttaattaccttttttatatgtattagtgtagagtagcattttatattttgatatcctccttatgcatagtttttcactttttattcctagtttttcgtttttaataagactttcaagaaatttattttattggccttttgaaaaaagcagctttagataaagtaagcagttctgctttcattttataatttatttctacttttgtttcattaatcttttcctccggcatgccttggattttgttgtgttactctttttctagaggctcgcattgtgtgtctggttcacttatgatcacgcttgcctacttttaagaatggaagaggggaggtggagggtggctgcacagtcgagggtgtgaggcagtcttgctctagccccaccatgccctcagcccgctgtggccacgctggttcctcaattgctggggcgtgcagtgtctgtaagggaggctactgatgccatccgaggaagatgtaaggtttcgtgtgggcagcgagagcctagcaggcatgtggggtgcccagcaaagggtaacagtggacagttgttgcctcattccacagagttttgattttttttttttttttaatggtcactccatcaacatcccccatggccagagcctgagctggtccccagagacacaggcattcagctgacagcctcgccttcacgctgctgctgttctcatgggggacaggcctcaggtggcaatgcacaaatcattagttaagggcagttgtgacagttaccaaggagtgtagtcccccgccccccgcccagtgaaaacagccctaaccaggggtggggacctttgggctctgacccgaagggtaggagaagctggaaggacagcattcctgtctgcgaaggcaggagcaaagctgccaggctatgaaggaaatggctggagcctgaagtcatgcaagctggggctggcagggacagggccaacttccaggcctgggggccaccatgaggattcaggacgtgacccccagggcacatgaaggccttccatctgtatttaagaaaagactttatcagacgagtatggtggctcacgcctgaatcttagcactttgggaggctgaggcaggtggatcacgaggtcaggagttcaataccagcctggccaatatggtaaaaccccatctctactaaaactacaaaaattagccaggcatggtggcgcacgcctgtagtcccagctactcgggaggctgaggcagaagaatcacttgaacccgggaggtggaggttacagtgagccaagatcgcgccactacactccagcctgggtgacagagtgagactccgtctcaaaaaaaccaaaagactttatcttatttcctatatgtttgtggtttcagtcctgatgtataatttgaccctagttagaatggttatctgaggaagtggcctgtacgatttctgcttttttaaatgtgtggctccctttcttcattgattaacgtatgattatttttataaatgttccatggcagtgggaagggattctctgtcacattccacatctggatcagttcctccccattttgttggtcaaatccgatctgccatatcctgtgtaatgacaagtgagttgcattctcaccgtcactcctggggtctctccgcttcccctgagctggctcagcagtctgctccatgtgttttgatgcagggtgacccattggtattcccgacactaacgcccccgtctgtggactgcttgctgcttgggcttcactgtgtctggtgttgacagtgcagacctaaaggtgtgcacacatgtgcacacacactccgctgtcttcttgtttgcactggacttaaatatctatgagggttattttcaactgctgaatttggaatgatttttatatcttttctgctttctgcccatgtacatgtgtttattttacactgttgtgattggtagttactatgtggggacacaattacttgggctgaaataatccacctgttgtggttggggtcctctggggcattccagggtgagaggttgtcactgccacctgggccatgtgggccggcaccagcattttgtggttacgaattctacagtcacaaatatctttgggcaaatccccttctatacctcaaggcagcttttggtttgcaaccccactggccagagggaagggccagtcacttggctctctcactgccctgcgccccagatggttctagggctgctgttttcccttggccctgccaacaccactgtttttacttctgctcattggctgagtgcagtggttcctggaagccagtggcacgtttccccgcgtagctcgcttatcccacagcacacacccaagggttctgttgctaacacgctgaattaattctttgctcatcttacagagtgtgttttgactgcccccatttctgaggccttgtaaggccagagctttgttgcttcatcggcaggttgggacttagatggccgtgaatgtttcctctctgctgctgcagtaagtaagtgcccgcaccatagtgtgtttggaggctgaagttgaagcgaggctgtgaggggagatggacgtgtgaggagggatgatggggcttgagcaaagtgggggagggggcaaaggcagttggcccaacacattccccacccctttgagaggtctgaggcctgcagacctggctcggagcccacctggtagtcctcagactgtgtgtgtgtgtgtgtgtgtgtgtgtgtgtgtgtgtgtgtgtgtgtgtgtgtgtgtgtaaaagagagaagttgtggagaaatggggggctgattctgctcagattcatcaggatgagtagaaggcacccagctctcaccctggcctgacatgtgtgtccctgagcaggttacagtcctctctgagcctctgcttcccatctggaccctgctgggcagggcttctgagctccttagcactagcaggaggggctccaggggccctccctccatggcagccaggacaggactctcaaatgaggacagcagagctcgtggggggctcccacggacccgccgtgggcccaggggaggcagagcctgagccaacagcagtggtgctgtggaccgtggatcctgagggtggcctggggcaagtaccggctgagggtccaggtgggctttgtgtacctttgggtcctggggccctggtgacttggactccaggttagagtcaagtgacaggagaaaggctggtggggccctgtgcttccgacttcatttcgagtgatggcagttcccaggaaggaatccacagctgacggtggctgacagatcagagaatggaaggcgaggcaggcgggcgtctgcgtgacctcaggtgcttggggcccagcagacccagagaaccatttccactaggccagggtgccggaagtgtccacaggtcttagattccctgttcagatgaaaagatttgtgcctttaatgataaaagtgatctgcatagagtcaaaaattcaagccatgggtataaaatgcaagtaaaatccctgccctcacctatcccaccctactacacagagatgtcctctcgagtttcctagactcactctggaaatttctgtatacacacagaagcttgtgcctctgctcgtgaaggcagagggagggagagctgaagggccagcaccttctcacctgtgggccccctcagtgctcggtcccagagcatgcaggactgtgcctcgtgttcagtttgctggtctgacttcatgctccttgggcaggatatgcatgtgccatgctaggagacatgtggatgtgaagctgggggacaatgtcccctggctatgcctttacaagggaagtaaggaaggtaggaggtgagcctgggagggagggagggaggcgcggagccgccgcaggtgtttcttttactgagtgcagcccatggccgcactcaggttttgcttttcaccttcccatctgtgaaagagtgagcaggaaaaagcaaaa

SEQ ID NO: 2 - DNA, 2199 bp, Homo sapiens
catttcgtcttagccacgcagaagtcgcgtgtctagtttgtttcgacgccggaccgcgtaagagacgatgatgttgggcacggaaggtggagagggattcgtggtgaaggtccggggcttgccctggtcttgctcggccgatgaagtgcagaggtttttttctgactgcaaaattcaaaatggggctcaaggtattcgtttcatctacaccagagaaggcagaccaagtggcgaggcttttgttgaacttgaatcagaagatgaagtcaaattggccctgaaaaaagacagagaaactatgggacacagatatgttgaagtattcaagtcaaacaacgttgaaatggattgggtgttgaagcatactggtccaaatagtcctgacacggccaatgatggctttgtacggcttagaggacttccctttggatgtagcaaggaagaaattgttcagttcttctcagggttggaaatcgtgccaaatgggataacattgccggtggacttccaggggaggagtacgggggaggccttcgtgcagtttgcttcacaggaaatagctgaaaaggctctaaagaaacacaaggaaagaatagggcacaggtatattgaaatctttaagagcagtagagctgaagttagaactcattatgatccaccacgaaagcttatggccatgcagcggccaggtccttatgacagacctggggctggtagagggtataacagcattggcagaggagctggctttgagaggatgaggcgtggtgcttatggtggaggctatggaggctatgatgattacaatggctataatgatggctatggatttgggtcagatagatttggaagagacctcaattactgtttttcaggaatgtctgatcacagatacggggatggtggctctactttccagagcacaacaggacactgtgtacacatgcggggattaccttacagagctactgagaatgacatttataattttttttcaccgctcaaccctgtgagagtacacattgaaattggtcctgatggcagagtaactggtgaagcagatgtcgagttcgcaactcatgaagatgctgtggcagctatgtcaaaagacaaagcaaatatgcaacacagatatgtagaactcttcttgaattctacagcaggagcaagcggtggtgcttacgaacacagatatgtagaactcttcttgaattctacagcaggagcaagcggtggtgcttatggtagccaaatgatgggaggcatgggcttgtcaaaccagtccagctacgggggcccagccagccagcagctgagtgggggttacggaggcggctacggtggccagagcagcatgagtggatacgaccaagttttacaggaaaactccagtgattttcaatcaaacattgcataggtaaccaaggagcagtgaacagcagctactacagtagtggaagccgtgcatctatgggcgtgaacggaatgggagggttgtctagcatgtccagtatgagtggtggatggggaatgtaattgatcgatcctgatcactgactcttggtcaaccttttttttttttttttttttttctttaagaaaacttcagtttaacagtttctgcaatacaagcttgtgatttatgcttactctaagtggaaatcaggattgttatgaagacttaaggcccagtatttttgaatacaatactcatctaggatgtaacagtgaagctgagtaaactataactgttaaacttaagttccagcttttctcaagttagttataggatgtacttaagcagtaagcgtatttaggtaaaagcagttgaattatgttaaatgttgccctttgccacgttaaattgaacactgttttggatgcatgttgaaagacatgcttttatttttttgtaaaacaatataggagctgtgtctactattaaaagtgaaacattttggcatgtttgttaattctagtttcatttaataacctgtaaggcacgtaagtttaagctttttttttttttaagttaatgggaaaaatttgagacgcaataccaatacttaggattttggtcttggtgtttgtatgaaattctgaggccttgatttaaatctttcattgtattgtgatttccttttaggtatattgcgctaagtgaaacttgtcaaataaatcctccttttaaaaactgca

SEQ ID NO: 3 - DNA, 21 bp, Artificial Sequence
  Description of Artificial Sequence Synthetic primer
aactttcgatggtagtcgccg

SEQ ID NO: 4 - DNA, 21 bp, Artificial Sequence
  Description of Artificial Sequence Synthetic primer
ccttggatgtggtagccgttt

SEQ ID NO: 5 - DNA, 20 bp, Artificial Sequence
  Description of Artificial Sequence Synthetic primer
gtgggatatgacagggacgc

SEQ ID NO: 6 - DNA, 20 bp, Artificial Sequence
  Description of Artificial Sequence Synthetic primer
ggcaggagcagctaatccaa

SEQ ID NO: 7 - DNA, 19 bp, Artificial Sequence
  Description of Artificial Sequence Synthetic primer
gagtggtaagaccaccacc

SEQ ID NO: 8 - DNA, 20 bp, Artificial Sequence
  Description of Artificial Sequence Synthetic primer
ggttttcctcaggatctaat

SEQ ID NO: 9 - DNA, 22 bp, Artificial Sequence
  Description of Artificial Sequence Synthetic primer
agtgctatctgaacctgtggtc

SEQ ID NO: 10 - DNA, 20 bp, Artificial Sequence
  Description of Artificial Sequence Synthetic primer
agtgctgggtcatccaaagg

SEQ ID NO: 11 - DNA, 20 bp, Artificial Sequence
  Description of Artificial Sequence Synthetic primer
ctccgcacggaccatgtatt

SEQ ID NO: 12 - DNA, 20 bp, Artificial Sequence
  Description of Artificial Sequence Synthetic primer
tcggactgaacgtggaaagg

SEQ ID NO: 13 - DNA, 20 bp, Artificial Sequence
  Description of Artificial Sequence Synthetic primer
tggggttttctggtgccttt

SEQ ID NO: 14 - DNA, 20 bp, Artificial Sequence
  Description of Artificial Sequence Synthetic primer
tagagggtcataccggcaca

SEQ ID NO: 15 - DNA, 20 bp, Artificial Sequence
  Description of Artificial Sequence Synthetic primer
gttatgggaccgcaccttca

SEQ ID NO: 16 - DNA, 20 bp, Artificial Sequence
  Description of Artificial Sequence Synthetic primer
cagtgaactggacccctgtc

SEQ ID NO: 17 - DNA, 20 bp, Artificial Sequence
  Description of Artificial Sequence Synthetic primer
tctggccagcttcgagaaag

SEQ ID NO: 18 - DNA, 20 bp, Artificial Sequence
  Description of Artificial Sequence Synthetic primer
agaactagacgtgcaaggcg

SEQ ID NO: 19 - DNA, 20 bp, Artificial Sequence
  Description of Artificial Sequence Synthetic primer
ccagagcagcatgagtggat

SEQ ID NO: 20 - DNA, 20 bp, Artificial Sequence
  Description of Artificial Sequence Synthetic primer
tagctgctgttcactgctcc

SEQ ID NO: 21 - DNA, 20 bp, Artificial Sequence
  Description of Artificial Sequence Synthetic primer
agcagacacacgtatgcgaa

SEQ ID NO: 22 - DNA, 20 bp, Artificial Sequence
  Description of Artificial Sequence Synthetic primer
gtggttcccaatccccacat

SEQ ID NO: 23 - DNA, 20 bp, Artificial Sequence
  Description of Artificial Sequence Synthetic primer
gtcctgacgacaacggacaa

SEQ ID NO: 24 - DNA, 20 bp, Artificial Sequence
  Description of Artificial Sequence Synthetic primer
agaatgaagaccaaccgccc

SEQ ID NO: 25 - DNA, 20 bp, Artificial Sequence
  Description of Artificial Sequence Synthetic primer
cacgtagacattccccgctt

SEQ ID NO: 26 - DNA, 20 bp, Artificial Sequence
  Description of Artificial Sequence Synthetic primer
ctgcctgcgtaactagaggg

SEQ ID NO: 27 - DNA, 20 bp, Artificial Sequence
  Description of Artificial Sequence Synthetic primer
ccagcaagagcacaagagga

SEQ ID NO: 28 - DNA, 20 bp, Artificial Sequence
  Description of Artificial Sequence Synthetic primer
acatggcaactgtgaggagg

SEQ ID NO: 29 - DNA, 20 bp, Artificial Sequence
  Description of Artificial Sequence Synthetic primer
gtgaagaccacctctcaggc

SEQ ID NO: 30 - DNA, 20 bp, Artificial Sequence
  Description of Artificial Sequence Synthetic primer
ctgtcgcagacactgtcact

SEQ ID NO: 31 - DNA, 20 bp, Artificial Sequence
  Description of Artificial Sequence Synthetic primer
gcgttgtcaacctcaccaac

SEQ ID NO: 32 - DNA, 20 bp, Artificial Sequence
  Description of Artificial Sequence Synthetic primer
gagtctccatgccgatccag

SEQ ID NO: 33 - DNA, 20 bp, Artificial Sequence
  Description of Artificial Sequence Synthetic primer
catggcatcgagagcaaacg

SEQ ID NO: 34 - DNA, 20 bp, Artificial Sequence
  Description of Artificial Sequence Synthetic primer
gacacggtgccagagaagaa

SEQ ID NO: 35 - DNA, 20 bp, Artificial Sequence
  Description of Artificial Sequence Synthetic primer
tgatggccagccactacaag

SEQ ID NO: 36 - DNA, 20 bp, Artificial Sequence
  Description of Artificial Sequence Synthetic primer
ccagcagtcaaaggggatga

SEQ ID NO: 37 - DNA, 52 bp, Artificial Sequence
  Description of Artificial Sequence Synthetic primer
ccagtgagcagagtgacgaggactcgagctcaagcttttttttttttttttt

SEQ ID NO: 38 - DNA, 18 bp, Artificial Sequence
  Description of Artificial Sequence Synthetic primer
gaggactcgagctcaagc

SEQ ID NO: 39 - DNA, 18 bp, Artificial Sequence
  Description of Artificial Sequence Synthetic primer
ccagtgagcagagtgacg

SEQ ID NO: 40 - DNA, 20 bp, Artificial Sequence
  Description of Artificial Sequence Synthetic primer
cagtcaggaagaaggcagca

SEQ ID NO: 41 - DNA, 20 bp, Artificial Sequence
  Description of Artificial Sequence Synthetic primer
gtgtgtagggaagggacagc

SEQ ID NO: 42 - DNA, 20 bp, Artificial Sequence
  Description of Artificial Sequence Synthetic primer
ccgaattggccgcaggtacc

SEQ ID NO: 43 - DNA, 42 bp, Artificial Sequence
  Description of Artificial Sequence Synthetic primer
taatacgactcactataggctgaatatatttatttgcatttg

SEQ ID NO: 44 - DNA, 47 bp, Artificial Sequence
  Description of Artificial Sequence Synthetic primer
taatacgactcactataggaaaaagtaaagactagaaattctttttg

SEQ ID NO: 45 - DNA, 19 bp, Artificial Sequence
  Description of Artificial Sequence Synthetic primer
cagcagcgacttcatccac

SEQ ID NO: 46 - DNA, 22 bp, Artificial Sequence
  Description of Artificial Sequence Synthetic primer
tagatgagaggcctgatcttta

SEQ ID NO: 47 - DNA, 18 bp, Artificial Sequence
  Description of Artificial Sequence Synthetic primer
gtgcctgcagccggcggg

SEQ ID NO: 48 - DNA, 42 bp, Artificial Sequence
  Description of Artificial Sequence Synthetic primer
cttgcggccgctaaccgtctttcttctatttcagactcctcc

SEQ ID NO: 49 - DNA, 39 bp, Artificial Sequence
  Description of Artificial Sequence Synthetic primer
cggttagcggccgcaagcaatgaagcatgcaggtctggg

SEQ ID NO: 50 - DNA, 31 bp, Artificial Sequence
  Description of Artificial Sequence Synthetic primer
cttgtcatttcatgtctctttaattaaagag

SEQ ID NO: 51 - DNA, 28 bp, Artificial Sequence
  Description of Artificial Sequence Synthetic primer
ggttaagcttagatgcatgctcgagcgg

SEQ ID NO: 52 - DNA, 37 bp, Artificial Sequence
  Description of Artificial Sequence Synthetic primer
taatacgactcactatagggtgggtctggcacgaagg

SEQ ID NO: 53 - DNA, 18 bp, Artificial Sequence
  Description of Artificial Sequence Synthetic primer
tgtgacggacgcaactgg

SEQ ID NO: 54 - DNA, 37 bp, Artificial Sequence
  Description of Artificial Sequence Synthetic primer
taatacgactcactataggaatacatggtccgtgcgg

SEQ ID NO: 55 - DNA, 18 bp, Artificial Sequence
  Description of Artificial Sequence Synthetic primer
cgcggcctgaggaagggg

SEQ ID NO: 56 - DNA, 37 bp, Artificial Sequence
  Description of Artificial Sequence Synthetic primer
taatacgactcactataggcgggaggagcgcggaggc

SEQ ID NO: 57 - DNA, 20 bp, Artificial Sequence
  Description of Artificial Sequence Synthetic primer
ccctagactcgccccacttg

SEQ ID NO: 58 - DNA, 37 bp, Artificial Sequence
  Description of Artificial Sequence Synthetic primer
taatacgactcactataggggacctaggcaacggcct

SEQ ID NO: 59 - DNA, 20 bp, Artificial Sequence
  Description of Artificial Sequence Synthetic primer
accggcaggagcttcatcac

SEQ ID NO: 60 - DNA, 37 bp, Artificial Sequence
  Description of Artificial Sequence Synthetic primer
taatacgactcactataggctttaatgccggggtctg

SEQ ID NO: 61 - DNA, 22 bp, Artificial Sequence
  Description of Artificial Sequence Synthetic primer
aagctaaatatggacctacgct

SEQ ID NO: 62 - DNA, 38 bp, Artificial Sequence
  Description of Artificial Sequence Synthetic primer
taatacgactcactataggagttttagaactggaaggg

SEQ ID NO: 63 - DNA, 25 bp, Artificial Sequence
  Description of Artificial Sequence Synthetic primer
tctttcttctatttcagactcctcc

SEQ ID NO: 64 - DNA, 41 bp, Artificial Sequence
  Description of Artificial Sequence Synthetic primer
taatacgactcactataggcaatgaagcatgcaggtctggg

SEQ ID NO: 65 - DNA, 30 bp, Artificial Sequence
  Description of Artificial Sequence Synthetic primer
ctgcaaaagaaaacaacaaaaaaactaaag

SEQ ID NO: 66 - DNA, 15 bp, Artificial Sequence
  Description of Artificial Sequence Synthetic oligonucleotide
aacacgtctatacgc

SEQ ID NO: 67 - DNA, 16 bp, Artificial Sequence
  Description of Artificial Sequence Synthetic oligonucleotide
ttacatgatgaccttt

SEQ ID NO: 68 - DNA, 20 bp, Artificial Sequence
  Description of Artificial Sequence Synthetic oligonucleotide
gcgactatacgcgcaatatg

SEQ ID NO: 69 - DNA, 20 bp, Artificial Sequence
  Description of Artificial Sequence Synthetic oligonucleotide
ctccagctccccccggcaac

SEQ ID NO: 70 - DNA, 91 bp, Artificial Sequence
  Description of Artificial Sequence Synthetic probe
attattatagatgagaggcctgatctttattgaaaacatattccaagtgttgaagacttttcattcttgtaagtccatacttattttcaaa

SEQ ID NO: 71 - DNA, 92 bp, Artificial Sequence
  Description of Artificial Sequence Synthetic probe
cagaacagcatagtctgttcattcattcattcaattcatgaattcattcacataattatccaatttcttgagcacctatttgatagtcactg

SEQ ID NO: 72 - DNA, 91 bp, Artificial Sequence
  Description of Artificial Sequence Synthetic probe
ttacttttttattacaaatggaatacgtatacttgcaaataattcagatactgtggaagagatcaaatgaattgcaaaagtgtccctcctc

SEQ ID NO: 73 - DNA, 92 bp, Artificial Sequence
  Description of Artificial Sequence Synthetic probe
ccttcaccactatctcccatggcatgcagagagagtaaccattatttgtgtgtccctccagaaatttttttattcaactactatttttttat

SEQ ID NO: 74 - DNA, 91 bp, Artificial Sequence
  Description of Artificial Sequence Synthetic probe
taaaaaagacttgaagtcaccctatgaagacaaaaaataatcacattaagtgtgaaagaacctattcttccagtacaggataagccatact

SEQ ID NO: 75 - DNA, 92 bp, Artificial Sequence
  Description of Artificial Sequence Synthetic probe
cacagttatgttatcaggtcacttgagttcaaagttttgtgttggcactagctaagtaaaggaaaacacctctgctttcattgttgagtttc

SEQ ID NO: 76 - DNA, 93 bp, Artificial Sequence
  Description of Artificial Sequence Synthetic probe
tatttcctgcaatggcgtccctgtcatatcccacaatggcctccctgccatttggatatcccttccatatcctgttgaaattactccctaata

SEQ ID NO: 77 - DNA, 92 bp, Artificial Sequence
  Description of Artificial Sequence Synthetic probe
ccttggtctttccattcctgggctaactaccatcaatctgagggctaacaatacaagtagaaaaagtatacatttgtcactgatcactgatc

SEQ ID NO: 78 - DNA, 92 bp, Artificial Sequence
  Description of Artificial Sequence Synthetic probe
tggtccctgatactggctcaggtaatgccactattgtcaagaagataccacttgtaaagtagatttaattttcattatattttaccatatgc

SEQ ID NO: 79 - DNA, 91 bp, Artificial Sequence
  Description of Artificial Sequence Synthetic probe
aagatattaaagaaatcatcatgacttagcctcatcaacagcattgctagatctgggatggaaaggaagagtataatcctggcagtcagga

SEQ ID NO: 80 - DNA, 92 bp, Artificial Sequence
  Description of Artificial Sequence Synthetic probe
agtagggaagggttttattcaggtcgtctgggctccataatatcccttgtgtatctgcagtctcctttgccatggatcaacacaataggaaa

SEQ ID NO: 81 - DNA, 92 bp, Artificial Sequence
  Description of Artificial Sequence Synthetic probe
tgtctctttaattaaagagttttgatttcagaggagggtacctgccttcgcagtccgcgtcctcgttccaccgaattggccgcaggtacccg

SEQ ID NO: 82 - DNA, 93 bp, Artificial Sequence
  Description of Artificial Sequence Synthetic probe
ccggctgcaggcacgtcctccagccggcggtccggggtggatgaagtcgctgctgccgaaaatccctgagcctccccgcgttggaccgacggc

SEQ ID NO: 83 - DNA, 93 bp, Artificial Sequence
  Description of Artificial Sequence Synthetic probe
cgcggcgtgcagactccgcacgctccccggccgagcccagacttgttcttctctcccttcccccattccccgagggacaaatttgtttaaact

SEQ ID NO: 84 - DNA, 93 bp, Artificial Sequence
  Description of Artificial Sequence Synthetic probe
aggaacgccactctatccgaccacggatcctcgtccattccgacactagggtctcaaccggtaagtcctggttgcgtgttcatctacgaccga

SEQ ID NO: 85 - DNA, 25 bp, Artificial Sequence
  Description of Artificial Sequence Synthetic oligonucleotide
caccgtactttaaaggtcgccgttt

SEQ ID NO: 86 - DNA, 25 bp, Artificial Sequence
  Description of Artificial Sequence Synthetic oligonucleotide
aaacaaacggcgacctttaaagtac

SEQ ID NO: 87 - RNA, 43 nt, Artificial Sequence
  Description of Artificial Sequence Synthetic oligonucleotide
ggggcgggccgguugccggggggagcuggagucacgugacggc

SEQ ID NO: 88 - RNA, 42 nt, Artificial Sequence
  Description of Artificial Sequence Synthetic oligonucleotide
uaaggggugaggggcaggagaauggguauggauggagguaga

SEQ ID NO: 89 - RNA, 42 nt, Artificial Sequence
  Description of Artificial Sequence Synthetic oligonucleotide
auacgugggaguggggugcugagcugggcagggugagaguau

SEQ ID NO: 90 - DNA, 22 bp, Artificial Sequence
  Description of Artificial Sequence Synthetic primer
tctgggagccatcttattatgc

SEQ ID NO: 91 - DNA, 23 bp, Artificial Sequence
  Description of Artificial Sequence Synthetic primer
gtttgtatcttggatgccacatt

SEQ ID NO: 92 - DNA, 21 bp, Artificial Sequence
  Description of Artificial Sequence Synthetic primer
ggtaattctgggagccatctt

SEQ ID NO: 93 - DNA, 21 bp, Artificial Sequence
  Description of Artificial Sequence Synthetic primer
cttgcagccatgatccaattc

SEQ ID NO: 94 - DNA, 21 bp, Unknown
  Description of Unknown shRNA 1 target sequence
gccattgcaggaaatataaga

SEQ ID NO: 95 - DNA, 25 bp, Unknown
  Description of Unknown shRNA 2 target sequence
cagctctcaattctgtgaaactcaa

SEQ ID NO: 96 - DNA, 16 bp, Artificial Sequence
  Description of Artificial Sequence Synthetic oligonucleotide
ttacatgatgaccttt

SEQ ID NO: 97 - DNA, 20 bp, Artificial Sequence
  Description of Artificial Sequence Synthetic oligonucleotide
gcgactatacgcgcaatatg

SEQ ID NO: 98 - DNA, 20 bp, Artificial Sequence
  Description of Artificial Sequence Synthetic oligonucleotide
ctccagctccccccggcaac

(19) United States
(12) Patent Application Publication
(10) Pub. No.: US 2024/0167098 A1
(43) Pub. Date: May 23, 2024

(54) METHODS FOR PREDICTING TREATMENT OUTCOME TO CHECKPOINT INHIBITORS IN CANCER

(71) Applicant: The Brigham and Women`s Hospital, Inc., Boston, MA (US)

(72) Inventors: Marco Mineo, Needham, MA (US); Ennio Antonio Chiocca, Weston, MA (US)

(21) Appl. No.: 18/283,266

(22) PCT Filed: Mar. 22, 2022
(86) PCT No.: PCT/US2022/021377

§ 371 Date: Sep. 21, 2023

Related U.S. Application Data
(60) Provisional application No. 63/169,392, filed on Apr. 1, 2021.
(60) Provisional application No. 63/163,935, filed on Mar. 22, 2021.

(51) Int. Cl. C12Q 1/6886; A61K 45/06; C07K 16/28

(52) U.S. Cl. CPC C12Q 1/6886; A61K 45/06; C07K 16/2827; C12Q 2600/106; C12Q 2600/158

(57) ABSTRACT

Described herein are methods of selecting a treatment for, and optionally treating, a subject who has a tumor and methods for determining is a subject who has a tumor is likely to benefit from an immunotherapy treatment. The methods disclosed herein can include the use of a long non-coding RNA, and/or a ribonucleoprotein, as biomarkers of patient response to immunotherapy treatment, such as an immune checkpoint inhibitor.

### CLAIM OF PRIORITY

This application claims the benefit of U.S. Provisional Patent Application Ser. No. 63/163,935, filed on Mar. 22, 2021 and U.S. Provisional Patent Application Ser. No. 63/169,392, filed on Apr. 1, 2021. The entire contents of the foregoing are hereby incorporated by reference.

### FEDERALLY SPONSORED RESEARCH OR DEVELOPMENT

This invention was made with government support under Grant No. CA236749 awarded by the National Institutes of Health. The government has certain rights in the invention.

### TECHNICAL FIELD

Provided herein, in part, are methods for identifying those subjects who are most likely to benefit from treatment with a checkpoint inhibitor based on levels of nuclear paraspeckle assembly transcript 1 (NEAT1) and/or heterogeneous nuclear ribonucleoprotein H1 (HNRNPH1), as well as methods for treating subjects with cancer using checkpoint inhibitors alone, or in combination with additional therapies.

### BACKGROUND

Cancer is among the leading causes of death worldwide. In 2018, there were 18.1 million new cases and 9.5 million cancer-related deaths worldwide. Furthermore, in 2020, an estimated 1,806,590 new cases of cancer will be diagnosed in the United States and 606,520 people will die from the disease. Estimated national expenditures for cancer care in the United States in 2018 were $150.8 billion. In future years, costs are likely to increase as the population ages and more people are treated for cancer. Costs are also likely to increase as new, and often more expensive, treatments are adopted as standards of care. (See, for example, National Cancer Institute. Cancer Statistics. cancer.gov/about-cancer/understanding). There is a need for better and more effective cancer treatments with more predictable outcomes and fewer or less severe toxic side effects.

### SUMMARY

While immune checkpoint inhibitors have produced durable antitumor activity in several different cancers, there is not a good biomarker to predict clinical response in cancer patients treated with immune checkpoint inhibitors. Several gene signatures have been developed as a multi-gene prognostic and predictive biomarkers and numerous gene signatures have been reported in the past decade (See, for example, Ayers et al. 2017. J Clin Invest. 2017 Aug. 1; 127(8):2930-2940). However, few advance to clinical development. One reason to not clinically develop gene signatures is that they can be tumor-type specific (i.e., not generic) and the type of assays used to determine gene signatures can affect the specific gene signature observed. Described herein is a gene signature that includes using a single long non-coding RNA gene, NEAT1, and/or a single coding gene, HNRNPH1, as biomarkers to predict clinical response to immune checkpoint inhibitors in multiple cancer types. This gene signature is simpler to measure.

Provided herein are methods of selecting a treatment for, and optionally treating, a subject who has a tumor including obtaining a sample, preferably comprising a plurality of cells, from the tumor of the subject; determining a tumor expression level of NEAT1 and/or HNRNPH1 in the sample; comparing the tumor expression level of NEAT1 and/or HNRNPH1 to a reference expression level of NEAT1 and/or HNRNPH1; selecting an immunotherapy treatment for the subject, e.g., for a subject who has a level of NEAT1 above the reference level and/or a level of HNRNPH1 below the reference level; and optionally administering the immunotherapy treatment to the subject. In some embodiments, the reference level is a level in a representative subject or cohort of subjects that is not sensitive to checkpoint inhibitors.

Also included herein are methods for determining if a subject who has a tumor is likely to benefit from an immunotherapy treatment including obtaining a sample preferably comprising a plurality of cells, from the tumor of the subject; determining an expression levels of NEAT1 and/or HNRNPH1 in the sample; and comparing the tumor expression levels of NEAT1 and/or HNRNPH1 to a reference expression level of the NEAT1 and/or HNRNPH1, wherein the tumor expression levels of NEAT1 and/or HNRNPH1 in comparison to the reference expression levels of NEAT1 and/or HNRNPH1 indicates whether the subject is likely to benefit from treatment with immunotherapy. In some embodiments, the reference level is a level in a representative subject or cohort of subjects that is not sensitive to checkpoint inhibitors.

In some cases, an increase in the expression level of NEAT1 compared to the reference expression level of NEAT1 indicates a likelihood of response to the immunotherapy treatment. In some cases, a decrease in the expression level of HNRNPH1 compared to the reference expression level of HNRNPH1 indicates a likelihood of response to the immunotherapy treatment. The methods can include selecting and optionally administering an immunotherapy to a subject who has a likelihood of response to the treatment.

In some cases, the sample is a fresh tumor sample. In some cases, the sample is a fixed tumor sample. The sample can be obtained, e.g., from a biopsy or surgical resection.

In some cases, the immunotherapy treatment comprises administration of an immune checkpoint inhibitor. In some cases, the immune checkpoint inhibitor is selected from an inhibitor of PD-1, an inhibitor of PD-L1, an inhibitor of CTLA-4, an inhibitor of Lag3, an inhibitor of CD40, an inhibitor of CD137, an inhibitor of OX40, and an inhibitor of Tim3. In some cases, the immune checkpoint inhibitor is an anti-PD-1 antibody.

In some cases, the method further comprises administering an additional treatment. In some cases, the additional treatment is selected from a second immune checkpoint inhibitor, a resection, a chemotherapy, and radiation. In some cases, the second immune checkpoint inhibitor is not an inhibitor of PD-1 or is not an inhibitor of PD-L1. In some cases, the second immune checkpoint inhibitor is an inhibitor or CTLA-4, an inhibitor of Lag3, or an inhibitor of Tim3.

In some cases, the method further comprises administering a second additional treatment. In some cases, the second additional treatment is chimeric antigen receptor (CAR) T cell (CAR T) therapy.

In some cases, the sample is from a glioblastoma cancer tumor or a carcinoma cancer tumor. In some cases, the carcinoma cancer tumor is a melanoma cancer tumor. In some cases, the subject is a mammal. In some cases, the mammal is a human or a non-human veterinary subject.

Also provided herein are methods to predict whether an immunotherapeutic intervention in a tumor-bearing cancer patient would likely be expected to result in a successful clinical response outcome including a) obtaining a tumor sample from tissue of the cancer patient; b) measuring the gene expression of one or more biomarkers in the tumor sample; and c) assessing whether the biomarker gene expression is changed in the tumor relative to the normal baseline expression of the biomarker in the tissue, wherein a change in the direction of gene expression of the biomarker predicts the cancer patient's response to immunotherapeutic intervention.

In some cases, the marker is a long non-coding RNA (lnc). In some cases, the lnc is NEAT1. In some cases, an increase in the expression of NEAT1 predicts a successful clinical response to immunotherapy.

In some cases, the immunotherapy is treatment or co-treatment with one or immune checkpoint inhibitors. In some cases, the immune checkpoint inhibitor is anti-PD-1. In some cases, the tumor is from glioblastoma or melanoma.

In some cases, the marker is a ribonucleoprotein. In some cases, the ribonucleoprotein is HNRNPH1. In some cases, a decrease in the gene expression of HNRNPH1 predicts a successful clinical response to immunotherapy.

In some cases, the immunotherapy is treatment, or co-treatment, with one or more immune checkpoint inhibitors. In some cases, the immune checkpoint inhibitor is anti-PD-1. In some cases, the tumor is from glioblastoma or melanoma.

Unless otherwise defined, all technical and scientific terms used herein have the same meaning as commonly understood by one of ordinary skill in the art. Methods and materials are described herein; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages will be apparent from the following detailed description and figures, and from the claims.

### DESCRIPTION OF DRAWINGS

FIGS. 1A-1C depict NEAT1 as a lncRNA highly expressed in patients who respond to anti-PD-1 therapy. (FIG. 1A) Volcano plot of differentially expressed lncRNAs (RNA-seq) between long-term survivor and short-term survivor glioblastoma (GBM) patients under anti-PD-1 therapy. (FIG. 1B) Volcano plot of differentially expressed lncRNAs (RNA-seq) between complete responder and non-responder melanoma patients under anti-PD-1 therapy. NEAT1 lncRNA is annotated. A total of 15,768 lncRNAs were surveyed in the analysis. (FIG. 1C) Venn diagram showing the lncRNAs commonly deregulated in glioblastoma and melanoma patients.

FIGS. 2A-2B depict NEAT1 predicting patient response to anti-PD-1 therapy. (FIGS. 2A-2B) Dot plots for the NEAT1 expression in patients with glioblastoma (FIG. 2A) and melanoma (FIG. 2B) under anti-PD-1 therapy.

FIGS. 3A-3B are Kaplan-Meier plots of overall survival in gliobastoma (FIG. 3A) and melanoma (FIG. 3B) based on NEAT1 expression.

FIGS. 4A-4B are graphs that show that HNRNPH1 predicts patient response to anti-PD-1 therapy in glioblastoma. (FIG. 4A) Dot plot for the HNRNPH1 expression in patients with glioblastoma under anti-PD-1 therapy. (FIG. 4B) Kaplan-Meier plot of overall survival in glioblastoma patients under anti-PD-1 therapy based on HNRNPH1 expression.

FIG. 5 shows an enriched Hallmark biochemical pathways (Liberzon et al., 2015) in melanoma between responders (including partial responders) to anti-PD-1 therapy versus non-responders (n=28, BH-adjusted p-values <0.05, generated by fgsea).

FIG. 6 shows PD-L1 expression between melanoma response groups. Boxplots comparing median-of-ratios normalized counts of PDL1 between anti-PD-1 complete responders, partial responders, and non-responders in melanoma (as defined by RECIST 1.1). P-values generated from Wilcoxon test.

FIGS. 7A-7D show boxplots comparing median-of-ratios normalized counts of IL10 (FIG. 7A), CDH1 (FIG. 7B), CCL7 (FIG. 7C), and AXL (FIG. 7D) between anti-PD-1 responders (including partial responders) and non-responders in melanoma (as defined by RECIST 1.1). P-values generated from Wilcoxon test. Data is derived from Hugo et al 2016 and reanalyzed.

FIG. 8 shows an enriched Hallmark biochemical pathways in glioblastoma between all neoadjuvant versus adjuvant anti-PD-1 patients (n=28, BH-adjusted p-values <0.05, generated by fgsea).

FIGS. 9A-9D show boxplots comparing median-of-ratios normalized counts of AGXT2L1 (FIG. 9A), WW1 (FIG. 9B), TOP2A (FIG. 9C), and TNC (FIG. 9D) between neoadjuvant (treatment group A) and adjuvant (treatment group B) glioblastoma patients from Cloughesy et al. (2019) and healthy astrocytes (treatment group C) from Zhang et al. (2016). P-values generated from Wilcoxon test.

FIG. 10 shows a Kaplan-Meir plot of GBM patients (n=28) with high (red, above median, counts>=12.5) versus low (blue) expression of INCR1. P-values by log-rank. Time measured in days.

FIG. 11 shows that INCR1 was associated with the interferon gamma response in melanoma. Hallmark biochemical pathway enrichment analysis comparing high (above median, counts >=39) versus low expression of primary INCR1 isoform in melanoma patients receiving anti-PD-1 therapy (BH-adjusted p-values <0.05).

FIG. 12 shows INCR1 was associated with interferon response in GBM. Hallmark biochemical pathway enrichment analysis comparing (A) high (above median, counts>=12.5) versus low expression of the primary INCR1 isoform in GBM patients receiving anti-PD-1 therapy (BH-adjusted p-values <0.05).

FIGS. 13A-13B show that NEAT1 was associated with responsiveness to anti-PD-1 in melanoma. Boxplots comparing median-of-ratios normalized counts of NEAT1 (FIG. 13A) between anti-PD-1 complete responders, partial responders, and non-responders in melanoma (as defined by RECIST 1.1) and (FIG. 13B) NEAT1 expression between patients not enriched for IPRES (N) and patients enriched for IPRES (Y), from Hugo et al. (2016). P-values generated from Wilcoxon test.

FIG. 14 shows that lncRNA expression was altered in GBM long-term survivors. Heatmap showing log 2(1+DESeq2 normalized expression counts) of 54 differentially expressed lncRNAs in long-term survivors from Cloughesy et al. (2019), with IDH-positive patients excluded (BH p-value <0.01).

FIGS. 15A-15B shows that NEAT1 predicted long-term survival in GBM and melanoma. Kaplan-Meir plot of (FIG. 15A) IDH-negative GBM patients (n=22) with high (red, above median, counts>=39172) vs low (blue) expression of NEAT1, and (FIG. 15B) melanoma patients (n=28) with high (red, counts>=44550, minimum expression in a complete responder patient) vs low (blue) expression of NEAT1. P-values by log-rank. Time measured in days.

FIG. 16 shows that NEAT1 expression was altered between neoadjuvant and adjuvant patients and healthy astrocytes. Boxplots comparing median-of-ratios normalized counts of NEAT1 between neoadjuvant (treatment group A) vs. adjuvant (treatment group B) GBM patients receiving anti-PD-1 and healthy adult astrocytes (treatment group C).

FIG. 17 shows that NEAT1 was associated with interferon response in melanoma. Hallmark biochemical pathway enrichment analysis comparing high (counts>=44550, minimum expression in a complete responder patient) to low expression of NEAT1 in melanoma patients receiving anti-PD-1 therapy. (BH-adjusted p-values <0.05).

FIG. 18 shows that NEAT1 was associated with interferon response in GBM. Hallmark biochemical pathway enrichment analysis comparing high expression (above median, counts>=39172) to low expression of NEAT1 in IDH-negative GBM patients (n=22) receiving anti-PD-1 therapy. (BH-adjusted p-values <0.05).

FIGS. 19A-19B shows that NEAT1 expression was associated with immune cell signatures in GBM. (FIG. 19A) Heatmap showing log 2(1+GSVA enrichment scores) for cell signatures (data derived from Regev et al., 2017 and reanalyzed). Signatures were selected after running fgsea and selecting for significant pathways (BH-adjusted p-value <0.01). (FIG. 19B) Heatmap showing log 2(1+DESeq2 normalized counts) for immune and cell cycle markers described by Cloughesy et al. (2019). IDH-negative GBM patients (n=22) from Cloughesy et al. (2019) separated by high (above median, counts>=39172) vs low NEAT1 expression.

FIG. 20 shows that NEAT1 altered immune and cell cycle marker expression in melanoma. Heatmap showing log 2(1+DESeq2 normalized counts) for immune and cell cycle markers described by Cloughesy et al. (2019). Melanoma patients (n=28) from Hugo et al. (2016) separated by high (counts>=44550) vs low NEAT1 expression.

FIG. 21 shows that NEAT1 was associated with altered gene expression in GBM. Heatmap showing log 2(1+DESeq2 normalized counts) for differentially expressed genes between high (above median, counts>=39172) vs low (below median) NEAT1 expression in IDH wildtype glioblastoma patients (n=22, BH-adjusted p-value <0.01).

FIGS. 22A-22C shows that HNRNPH1 was a binding partner of INCR1. FIG. 22A is an exemplary methodology workflow. FIG. 22B is a graph of RNA enrichment of INCR1. FIG. 22C is a graph of the identity of enriched RNAs, with HNRNPH1 as the highest enriched RNA.

FIGS. 23A-23C shows that HNRNPH1 binds INCR1 in the proximal intron. FIG. 23A is an exemplary methodology workflow of determining molecular interactions. FIG. 23B is a chart of RNA enrichment across the INCR1 gene. FIG. 23C is a blot confirming enrichment in the proximal region of INCR1.

FIGS. 24A-24C shows that HNRNPH1 binds PD-L1 and JAK2. FIG. 24A is a graph INCR1 fold enrichment with control or HNRNPH1 antibodies. FIG. 24B is a graph of PD-L1 enrichment and JAK2 enrichment. FIG. 24C is a graph of RNA enrichment of control or INCR1 antisense oligos.

FIGS. 25A-25C show that HNRNPH1 was a negative regulator of PH-L1 and JAK2. FIG. 25A is a series of graphs of relative RNA expression levels of HNRNPH1 (left), PD-L1 (middle), or JAK2 (right). FIG. 25B is a series of blots of PD-L1, JAK2, HNRNPH1, and beta-actin in the presence of the indicated silencing RNAs in the presence or absence of IFN-gamma. FIG. 25C is a chart of gene expression of either PD-L1 or HNRNPH1 in glioblastoma samples from the TCGA.

FIGS. 26A-26B show that INCR1 interfered with HNRNPH1 binding to PD-L1 and JAK2. FIG. 26A is a series of graphs of Fnorm (%) on the y-axis and the log concentration of HNRNPH1 molarity on the x-axis. Enzymatic binding activity between HNRNPH1 and INCR1 (top), PD-L1 (middle), and JAK2 (bottom) was determined. FIG. 26B is a plot of an EMSA analysis of the effect of INCR1 RNA fragment on the ability of HNRNPH1 to bind to radiolabeled PD-L1 (left) or JAK2 (right) RNA fragments (50 nM). No protein was added to lanes 1 and 8. HNRNPH1 was added at a concentration of 0.65 mM (lanes 2 and 9), 3.25 mM (lanes 3 and 10), and 6.5 mM (lanes 4-7 and 11-14). INCR1 was added at a molar ratio of 1:1 (lanes 5 and 12), 1:5 (lanes 6 and 13), and 1:10 (lanes 7 and 14).

FIGS. 27A-27D shows that INCR1 binds HNRNPH1 to allow PD-L1 and JAK2 expression. FIG. 27A is an exemplary workflow schematic where H1 represents HNRNPH1. FIG. 27B is a blot of an RNA pull-down analysis of biotinylated fragment 4 (F4) in the presence of increasing concentrations of antisense oligonucleotide targeting HNRNPH1 binding site (ASO H1B). No RNA fragment was added in the lanes marked “-”. FIG. 27C is a blot of a RNA pull-down assay with biotinylated INCR1 fragment 4 (F4) in the presence of antisense oligonucleotide control (ASO NC) or targeting HNRNPH1 binding site (ASO H1B). FIG. 27D is a blot of an RNA pull-down assay with biotinylated INCR1 fragment 4 (F4) in the presence of antisense oligonucleotide control (ASO NC) or targeting HNRNPH1 binding site (ASO H1B).

FIG. 28 is a Gene Ontology analysis of the genes bound to HNRNPH1 identified by eCLIP.

FIGS. 29A-29E show that HNRNPH1 negatively regulated interferon-stimulated genes.

FIG. 30 is a graph of the relative PD-L1 variant 1 to variant 4 ratio under various silencing RNA treatments with and without interferon-gamma.

FIG. 31 is PD-L1 read density in reads per million usable, indicating binding sites enriched in HNRNPH1 compared to control IgG.

FIG. 32A is a schematic of alternative splicing of PD-L1 intron 5. FIG. 32B is a graph of the percentage of unspliced PD-L1 intron 5 to spliced PD-L1 of intron 5 after exposure to various silencing RNAs.

FIG. 33 is a graph of the HNRNPH1 expression in normal and glioblastoma subjects.

FIGS. 34A-34B shows that HNRNPH1 expression levels predicted patient response to immune checkpoint therapy in glioblastoma patients.

FIGS. 35A-35C shows that silencing HNRNPH1 improves CAR T cell activity. FIG. 35A is an exemplary method schematic. FIG. 35B is a collection of exemplary fluorescence microscopy images of GBM62 and T cells exposed to various silencing RNAs and CAR T therapies. FIG. 35C is a graph of tumorsphere area.

FIGS. 36A-36B showed that silencing HNRNPH1 in combination with anti-PD-1 therapy improved CAR T cell activity. FIG. 36A is a fluorescent microscopy image of cells exposed to silencing RNAs and either IgG or anti-PD-L1 (aPD-L1).

FIG. 37 shows that HNRNPH1 expression levels predicts patient response to immune checkpoint therapy in melanoma patients. Data is from Riaz et al 2017.

### DETAILED DESCRIPTION

Described herein are methods of selecting a treatment for, and optionally treating, a subject who has a tumor and methods for determining is a subject who has a tumor is likely to benefit from an immunotherapy treatment. The methods disclosed herein use a single long non-coding RNA, and/or a single ribonucleoprotein, as biomarkers of patient response to immunotherapy treatment, such as an immune checkpoint inhibitor.

Cancers occur when cells abnormal or damaged cells grow and multiply in an uncontrolled or abnormal manner. These cells may form tumors. Tumors can be cancerous or not cancerous (benign). Cancerous tumors can invade nearby tissues and metastasize to other places in the body to form new tumors. Cancerous tumors may also be called malignant tumors. Many cancers form solid tumors, but cancers of the blood, such as leukemias, generally do not. Benign tumors do not metastasize. When removed, benign tumors usually do not grow back, whereas cancerous tumors may return.

Immune checkpoint inhibitors have improved cancer treatment (1, 2). These therapies have been developed based on targeting the ability of cancers to evade anti-tumor immunity by the upregulation of immune checkpoint molecules, such as programmed cell death 1 ligand 1 (PD-L1) (3, 4). Expression of PD-L1 within the tumor microenvironment inhibits the anti-tumor immune response through the binding of the immune checkpoint receptor PD-1 expressed on T cells (5). Immune checkpoint inhibitors that target the PD-1/PD-L1 pathway can be less toxic than standard chemotherapy and can produce both durable tumor regression and overall survival benefits in several tumors, including non-small cell lung cancer (NSCLC) and melanoma (6-8). However, not all patients respond to these therapies and there is currently no biomarker that reliably predicts clinical outcomes.

Described herein are methods of predicting clinical responses to immunotherapy and selecting treatment for a subject (e.g. selecting an immunotherapy) using the expression levels of long non-coding RNA, NEAT1, and/or the ribonucleoprotein, HNRNPH1 as indicators of treatment success (See Table 1 for sequences).

### Cancer Biomarkers

Long non-coding RNAs (lncRNAs) are a class of transcripts longer than 200 nucleotides that lack of protein-coding potential. These molecules can influence several biological processes, such as cell proliferation, migration and immune response. LncRNAs can be categorized in terms of length, function, location, and targeting mechanism. According to their position in the genome relative to protein-coding genes, they can be classified as sense, antisense, bidirectional, intronic, intergenic, and enhancer lncRNAs, with their functions dependent on their position. Simultaneously, lncRNAs can be sorted into bait, scaffold, signal, and guide lncRNAs based on their function mechanisms. lncRNAs can encode small peptides to fine-tune general biological processes in a tissue-specific manner (See, for example, Larkin et al. 2015. N Engl J Med. 373: 23-34; Sharpe et al. 2007. Nat Immunol. 8: 239-245; and Chen et al 2021. Acta Pharm Sin B. 2021 February; 11(2): 340-354). One lncRNA is NEAT1 (See, for example, Clemson et al, 2009. Mol. Cell. 33:6, 717-726).

Heterogeneous nuclear ribonucleoproteins (hnRNPs) are a family of RNA-binding proteins that play a central role in several aspects of RNA metabolism and global gene expression (See, for example, Geuens et. al. Hum Genet. 2016; 135: 851-867). Many ribonucleoproteins (RNPs) assemble on to newly created transcripts in the nucleus of a eukaryotic cell. Among these RNPs are the heterogeneous nuclear ribonucleoproteins (hnRNPs). They assist in controlling the maturation of newly formed heterogeneous nuclear RNAs into messenger RNAs (mRNAs), stabilize mRNA during their cellular transport and control their translation. hnRNPs can act as key proteins in the cellular nucleic acid metabolism.

Expression of both lncRNAs and hnRNPs may be important in cancer progression. For example, deregulation of lncRNA expression has been implicated in cancer progression, suggesting lncRNAs as master drivers of carcinogenesis (12-15) and several lncRNAs have been shown to function through the interaction with different heterogeneous ribonucleoproteins (hnRNPs) (16, 17). Specifically, the lncRNA INCR1 regulates tumor interferon signaling functioned through interacting with the ribonucleoprotein HNRNPH1 and silencing INCR1 sensitized tumor cells to cytotoxic T cell-mediated killing in vitro, and improved CAR T cell therapy in vivo (18). Additionally, HnRNP expression levels can be altered in several cancers, and may influence tumor development and metastasis (See, for example, Liu et al. 2015. Gene. 2015; 576:791-797; Loh et al. 2015. Oncol Rep. 2015 September; 34(3):1231-8; and Jean-Philippe et al. 2013. Int J Mol Sci. 2013 Sep. 16; 14(9):18999-9024).

### Methods of Identifying and Selecting Subjects for Treatment

Described herein are methods to select a treatment for, and optionally treat, a subject who has a tumor and methods for determining if a subject who has a tumor is likely to benefit from an immunotherapy treatment. The methods include obtaining a sample from a patient, determining an expression level of a biomarker gene, comparing the tumor expression level to a reference expression level in non-tumor cells, and selecting an immunotherapy treatment.

Tumor samples can be obtained from a subject identified as having or suspected of having cancer. In some cases, the sample is from a biopsy, e.g., a tissue biopsy, core needle biopsy, or fine needle aspirate (FNA); or from a lumpectomy or resection. In some cases, a sample comprises a plurality of cells from the tumor. In some cases, the sample is from tumor of the subject. In some cases, a sample comprising a plurality of cells from the tumor of the subject is obtained. Various methods known within the art can be used for the identification and/or isolation and/or purification of a biological marker from a sample. An “isolated” or “purified” biological marker that is substantially free of cellular material or other contaminants from the cell or tissue source from which the biological marker is derived, i.e., partially or completely altered or removed from the natural state through human intervention. For example, nucleic acids contained in a sample can be first isolated according to standard methods, for example using lytic enzymes, chemical solutions, or isolated by nucleic acid-binding resins following the manufacturer's instructions.

Determining an expression level (e.g., an RNA expression level) of a gene or other non-coding RNA, such as a biomarker, can use methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., gene chips, as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Hardiman, Microarrays Methods and Applications: Nuts & Bolts, DNA Press, 2003), can be used to detect the presence and/or level of NEAT1 and/or HNRNPH1.

Measurement of the level of a biomarker can be direct or indirect. For example, the abundance levels of NEAT1 and/or HNRNPH1 can be directly determined or quantified. Alternatively, the amount of a biomarker can be determined indirectly by measuring abundance levels of cDNA, amplified RNAs, or by measuring quantities or activities of RNAs, or other molecules that are indicative of the expression level of the biomarker. In some embodiments, a technique suitable for the detection of alterations in the structure or sequence of nucleic acids, such as the presence of deletions, amplifications, or substitutions, can be used for the detection of biomarkers in any of the methods or composition described herein.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such actin B (ACTB (e.g., NM_001101.4)), glyceraldehyde dehydrogenase (GAPDH (e.g., NM_002046.6)) and RPLPO (36B4, e.g., NM_001002.3), are most commonly used.

Gene arrays can be prepared by selecting probes that comprise a polynucleotide sequence, and then immobilizing such probes to a solid support or surface. For example, the probes can comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

Comparing the tumor expression level of a biomarker to a reference expression level of a biomarker can be performed. For example, a reference expression level can be determined from a plurality of non-tumor cells. The non-tumor cells can be derived from the subject who has a tumor or can be derived from a different subject. The reference expression level can also be determined as an average expression level in non-cancerous cells in the scientific literature. In some cases, the tumor expression level of the biomarkers is compared to a reference expression level of the biomarkers. In some cases, the tumor expression level of NEAT1 and/or HNRNPH1 is compared to a reference expression level of NEAT1 and/or HNRNPHlin a plurality of non-tumor cells.

Selecting an immunotherapy treatment for the subject can include an assessment of the comparison of the tumor expression level of a biomarker (e.g. NEAT1 and/or HNRNPH1) to a reference expression level of the biomarker (e.g. NEAT1 and/or HNRNPH1). In some cases, an increase in the expression level of NEAT1 compared to the reference expression level of NEAT1 indicates a high likelihood of response to the immunotherapy treatment. In some cases, a decrease in the expression level of HNRNPH1 compared to the reference expression level of HNRNPH1 indicates a high likelihood of response to the immunotherapy treatment. In some cases, an decrease in the expression level of NEAT1 compared to the reference expression level of NEAT1 indicates a low likelihood of response to the immunotherapy treatment. In some cases, an increase in the expression level of HNRNPH1 compared to the reference expression level of HNRNPH1 indicates a low likelihood of response to the immunotherapy treatment.

Methods herein can optionally include administering an immunotherapy treatment to the subject. In some cases, the immunotherapy treatment is an immune checkpoint inhibitor.

### Cancers and Tumors

Cancers and tumors can be detected with various diagnostic means such as cancer screenings and can include visual inspection, physical inspection, and laboratory tests (for example, histopathology or pap smears). In some cases, the cancer is detected by the identification of a tumor. In some cases, the cancer is detected by the identification of cells or a plurality of cells within or from the tumor. In some cases, the tumor is a fresh tumor, a frozen tumor (e.g. previously biopsied), a biopsy of a tumor, or a fixed sample of a tumor (e.g., paraffin fixed sample or a formalin fixed sample, such as a tumor sample).

A non-limiting list of cancers that a subject may be identified as having includes bladder cancer, breast cancer, colon cancer, rectal cancer, endometrial cancer, kidney cancer, leukemia, liver cancer, lung cancer, melanoma, non-hodgkin lymphoma, pancreatic cancer, prostate cancer, thyroid cancer, brain cancer, skin cancer, and carcinoma.

Brain cancers can include gliomas (e.g., astrocytomas, oligodendrogliomas, ependymomas, choroid plexus papillomas, glioblastomas), meningiomas, pituitary adenomas, vestibular schwannomas, and primitive neuroectodermal tumors (medulloblastomas). In some cases, the tumor is from a subject identified as having glioblastoma. In some cases, the tumor is from a glioblastoma cancer tumor. In some cases, the tumor is from a subject identified as having glioblastoma and modified expression of NEAT1 and/or HNRNPH1. In some cases, the tumor is from a glioblastoma cancer tumor and has modified expression of NEAT1 and/or HNRNPH1. In some cases, the tumor is from a subject identified as having glioblastoma and an increased expression of NEAT1 and/or a decreased expression of HNRNPH1. In some cases, the tumor is from a glioblastoma cancer tumor and has an increased expression of NEAT1 and/or a decreased expression of HNRNPH1. In some cases, the tumor is from a glioblastoma cancer tumor and has modified expression of NEAT1 and/or HNRNPH1 compared to the reference expression levels of NEAT1 and/or HNRNPH1 in non-tumor cells. In some cases, the tumor is from a subject identified as having glioblastoma and an increased expression of NEAT1 and/or a decreased expression of HNRNPH1 compared to the reference expression levels of NEAT1 and/or HNRNPH1 in non-tumor cells. In some cases, the tumor is from a glioblastoma cancer tumor and has an increased expression of NEAT1 and/or a decreased expression of HNRNPH1 compared to the reference expression levels of NEAT1 and/or HNRNPH1 in non-tumor cells.

Carcinoma cancers often start in cells that make up the skin or the tissue lining organs, such as the liver or kidneys, and do not often start in bone, blood vessels, the immune system cells, the brain, or the spinal cord. Carcinoma cancers can include basal cell carcinoma, squamous cell carcinoma, renal cell carcinoma, ductal carcinoma in situ (DCIS), invasive ductal carcinoma, adenocarcinoma, and melanoma. In some cases, the sample is from tumor of the subject. In some cases, the tumor is from a subject identified as having carcinoma. In some cases, the tumor is from a subject identified as having melanoma. In some cases, the sample is from a carcinoma cancer tumor. In some cases, the sample is from a melanoma cancer tumor. In some cases, the tumor is from a subject identified as having carcinoma and as having an increased expression of NEAT1 and/or a decreased expression of HNRNPH1. In some cases, the sample is from a carcinoma cancer tumor and has an increased expression of NEAT1 and/or a decreased expression of HNRNPH1. In some cases, the tumor is from a subject identified as having melanoma and as having an increased expression of NEAT1 and/or a decreased expression of HNRNPH1. In some cases, the sample is from a melanoma cancer tumor and has an increased expression of NEAT1 and/or a decreased expression of HNRNPH1.

In some cases, the tumor is from a subject identified as having carcinoma and as having modified expression of NEAT1 and/or HNRNPH1 compared to the reference expression levels of NEAT1 and/or HNRNPH1 in non-tumor cells. In some cases, the sample is from a carcinoma cancer tumor and has modified expression of NEAT1 and/or HNRNPH1 compared to the reference expression levels of NEAT1 and/or HNRNPH1 in non-tumor cells. In some cases, the tumor is from a subject identified as having carcinoma and as having an increased expression of NEAT1 and/or a decreased expression of HNRNPH1 compared to the reference expression levels of NEAT1 and/or HNRNPH1 in non-tumor cells. In some cases, the sample is from a carcinoma cancer tumor and has an increased expression of NEAT1 and/or a decreased expression of HNRNPH1 compared to the reference expression levels of NEAT1 and/or HNRNPH1 in non-tumor cells. In some cases, the tumor is from a subject identified as having melanoma and as having an increased expression of NEAT1 and/or a decreased expression of HNRNPH1 compared to the reference expression levels of NEAT1 and/or HNRNPH1 in non-tumor cells. In some cases, the sample is from a melanoma cancer tumor and has an increased expression of NEAT1 and/or a decreased expression of HNRNPH1 compared to the reference expression levels of NEAT1 and/or HNRNPH1 in non-tumor cells.

### Subjects

The present methods can be used in the selection and treatment of subjects with cancer, e.g., carcinoma, sarcoma, metastatic disorders or hematopoietic neoplastic disorders, e.g., leukemia. In some embodiments, the subjects have or are suspected to have a cancer of the brain (e.g., glioma, e.g., glioblastoma) or a carcinoma (e.g., solid tumors of epithelial origin, e.g., cancer of the breast, lung, ovary, colon, kidney, prostate, or pancreas), sarcoma, or melanoma. Methods for identifying or diagnosing subjects with a cancer are known in the art, and can include biopsy, imaging, and biomarker analysis.

For any of the methods described herein, the subject can be a mammal. In some cases, mammal is a human or a non-human veterinary subject (e.g. dog, cat, horse, pig, cow, goat, sheep, llama, donkey, etc).

### Immunotherapy Treatment

Methods described herein can include selecting an immunotherapy treatment for a subject, and optionally administering the immunotherapy treatment to the subject. Immunotherapy treatments are a type of biological therapy that uses substances made from living organisms to prevent, slow, or destroy cancerous growth such a tumors. Immunotherapies can, for example, help the immune system better identify cancerous cells or increase the immune system's response to cancer. Biomarkers can be useful to identify potential responses to immunotherapy treatment. Immunotherapy treatments can include immune checkpoint inhibitors, T-cell transfer therapy (e.g. tumor-infiltrating lymphocytes (or TIL) therapy or CAR T-cell therapy), monoclonal antibodies, vaccines (e.g. talimogene laherparepvec (T-VEC, or Imlygic®), and immune system modulators (e.g. cytokines, such as interferons or interleukins; hematopoietic growth factors such as erythropoietin, IL-11, granulocyte-macrophage colony-stimulating factor (GM-CSF) or granulocyte colony-stimulating factor (G-CSF); BCG; and immunomodulatory drugs (i.e. biological response modifiers, such as thalidomide (Thalomid®), lenalidomide (Revlimid®), pomalidomide (Pomalyst®), or imiquimod (Aldara®, Zyclara®)).

Immune Checkpoint Inhibitors.

Immune checkpoints are a normal part of the immune system. Their role is to prevent an immune response from being so strong that it destroys healthy cells in the body. Immune checkpoints engage when proteins on the surface of immune cells called T cells recognize and bind to partner proteins on other cells, such as some tumor cells. These proteins are called immune checkpoint proteins. When the checkpoint and partner proteins bind together, they send an “off” signal to the T cells. This can prevent the immune system from destroying the cancer.

Immunotherapy drugs called immune checkpoint inhibitors work by blocking checkpoint proteins from binding with their partner proteins. This prevents the “off” signal from being sent, allowing the T cells to kill cancer cells.

Currently approved immune checkpoint blockers are monoclonocal antibodies (mAbs) that target the programmed cell death protein 1 (PD-1)/PD-L1/2 or cytotoxic T-lymphocyte-associated protein 4 (CTLA-4) pathways, and agents targeting other pathways are in clinical development (including CD40, OX40, Tim-3, and LAG-3) (See, e.g., Leach et al., Science 271, 1734-1736 (1996); Pardoll, Nat. Rev. Cancer 12, 252-264 (2012); Topalian et al., Cancer Cell 27, 450-461 (2015); Mahoney et al., Nat Rev Drug Discov 14, 561-584 (2015)). The present methods can include the administration of checkpoint inhibitors such as antibodies including anti-CD137 (BMS-663513); anti-PD-1 (programmed cell death 1) antibodies (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538)); anti-PDL1 (programmed cell death ligand 1) or anti-PDL2 (e.g., BMS-936559, MPDL3280A, atezolizumab, avelumab and durvalumab); or anti-CTLA-4 (e.g., ipilumimab or tremelimumab). See, e.g., Kruger et al., “Immune based therapies in cancer,” Histol Histopathol. 2007 June;22(6):687-96; Eggermont et al., “Anti-CTLA-4 antibody adjuvant therapy in melanoma,” Semin Oncol. 2010 October;37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August;33(6):570-90; Moschella et al., “Combination strategies for enhancing the efficacy of immunotherapy in cancer patients,” Ann N Y Acad Sci. 2010 April;1194:169-78; Ganesan and Bakhshi, “Systemic therapy for melanoma,” Natl Med J India. 2010 January-February;23(1):21-7; Golovina and Vonderheide, “Regulatory T cells: overcoming suppression of T-cell immunity.” Cancer J. 2010 July-August;16(4):342-7.

In some cases, in any of the methods described herein the immune checkpoint inhibitor is selected from an inhibitor of PD-1, an inhibitor of PD-L1, an inhibitor of CTLA-4, an inhibitor of Lag3, an inhibitor of CD40, an inhibitor of CD137, an inhibitor of OX40, and an inhibitor of Tim3. In some cases, the immune checkpoint inhibitor is an inhibitor of PD-1. In some cases, the immune checkpoint inhibitor is an inhibitor of PD-L1. In some cases, the immune checkpoint inhibitor is an anti-PD-1 antibody. In some cases, the immune checkpoint inhibitor is an anti-PD-L1 antibody.

In addition to or as an alternative to checkpoint inhibitors, the methods described herein can be used to predict benefit from and select treatment with any type of immunotherapy whose mechanism is CD8 T cell-mediated (e.g., vaccines, dendritic cell-based immunizations, or adoptively transferred anti-tumor CD8 T cells, etc); see, e.g., Durgeau et al., Front Immunol. 2018; 9:14).

### Reference Expression Levels

Suitable reference values, such as reference expression levels can be determined using methods known in the art, e.g., using standard clinical trial methodology and statistical analysis. The reference values can have any relevant form. In some cases, the reference value (e.g. the reference expression level) is determined from a plurality of non-tumor cancer cells, optionally from the subject identified as having a tumor or from a different subject. In some cases, the reference comprises a predetermined value for a meaningful level of the biomarker, e.g., a reference corresponding to a level of NEAT1 and/or HNRNPH1 in a representative subject or cohort of subjects that is sensitive to checkpoint inhibitors, and/or a level of NEAT1 and/or HNRNPH1 in a representative subject or cohort of subjects that is not sensitive to checkpoint inhibitors.

A predetermined reference expression level can depend upon the particular population of subjects (e.g., human subjects) selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

The predetermined or reference level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with response to checkpoint inhibitors in one defined group is a fold higher, or lower, (e.g., approximately 2-fold, 4-fold, 8-fold, 16-fold or more) than the response to checkpoint inhibitors in another defined group. It can be a range, for example, where a population of subjects (e.g., control subjects) is divided equally (or unequally) into groups, such as a low-likelihood of response group, a medium-likelihood of response group and a high-likelihood of response group, or into quartiles, the lowest quartile being subjects with the lowest likelihood of response and the highest quartile being subjects with the highest likelihood of response, or into n-quantiles (i.e., n regularly spaced intervals) the lowest of the n-quantiles being subjects with the lowest likelihood of response and the highest of the n-quantiles being subjects with the highest likelihood of response.

### Methods for Treating Subjects

The methods described herein can include the use of pharmaceutical compositions comprising an immunotherapy treatment such as an immune checkpoint inhibitor, e.g., anti-PD1, as the active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, NJ) or phosphate buffered saline (PBS). In some cases, the composition should be sterile and should be fluid to the extent that easy syringability exists. It should be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the preferred particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the preferred amount in an appropriate solvent with one or a combination of ingredients enumerated above followed by filtered sterilization. Generally, dispersions are prepared by incorporating the active compound into a sterile vehicle, which contains a basic dispersion medium and any other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In some cases, any of the methods described herein further include administering an additional treatment. Additional treatments can include a second immune checkpoint inhibitor, resection, chemotherapy, and radiation. In some cases, the second immune checkpoint inhibitor is not an inhibitor of PD-1 and/or not an inhibitor of PD-L1. In some cases, the second immune checkpoint inhibitor is an inhibitor or CTLA-4, an inhibitor of Lag3, or an inhibitor of Tim3.

### EXAMPLES

Additional description is provided in the following examples, which do not limit the scope of any of the claims.

### Example 1—Nuclear Non-Coding RNAs and Ribonucleoproteins as Biomarkers of Response to Immunotherapy

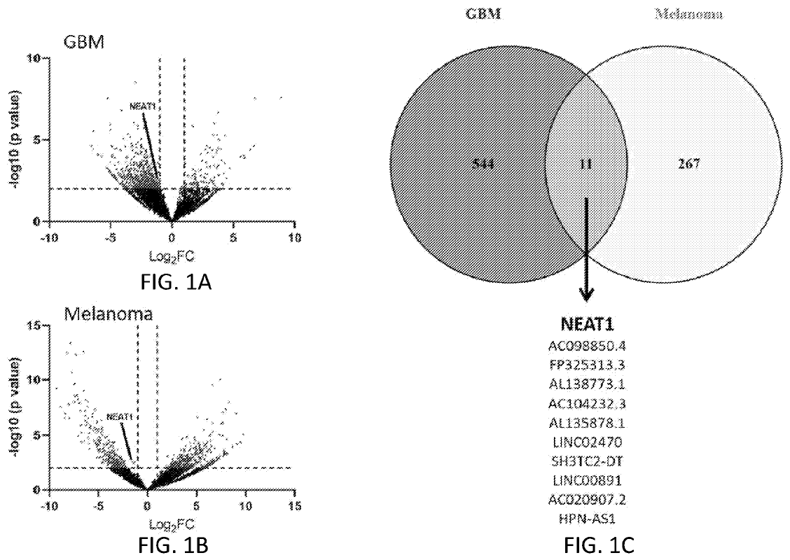

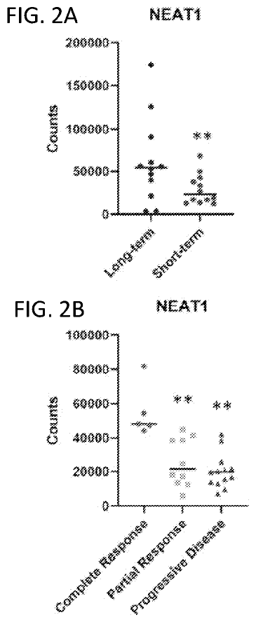

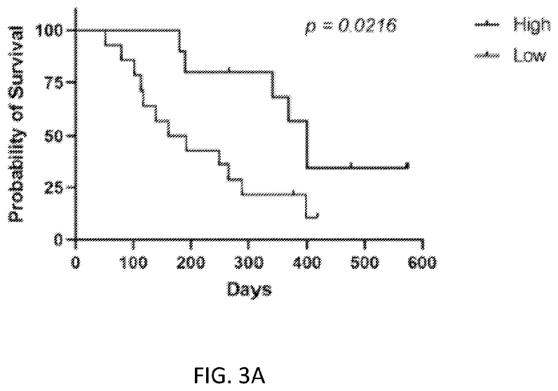

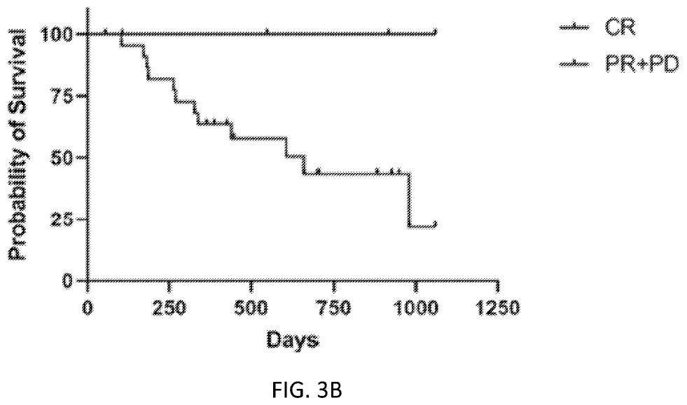

To identify lncRNAs that could be used as biomarkers to predict cancer patient response to immune checkpoint blockade, a whole-transcriptome analysis was conducted in patients undergoing anti-PD-1 therapy with either glioblastoma or melanoma. 555 lncRNAs were identified as deregulated in glioblastoma patients (p<0.01, FC>2) who showed longer survival in response to anti-PD-1 treatment and 278 lncRNAs deregulated in melanoma patients who showed complete response (FIG. 1A-C). 11 lncRNAs were found to be commonly deregulated in glioblastoma and melanoma (FIGS. 1A-C). Among the most significantly deregulated genes, NEAT1 was identified as a lncRNA upregulated in both in glioblastoma patients who showed longer survival and melanoma patients who showed complete response (FIGS. 2A-2B and 3A-3B). Pathway analysis showed that high levels of NEAT1 correlated with the expression of interferon gamma-related genes in both glioblastoma and melanoma patients, which was previously shown to be a marker of patient response to immunotherapy (FIGS. 17 and 18).

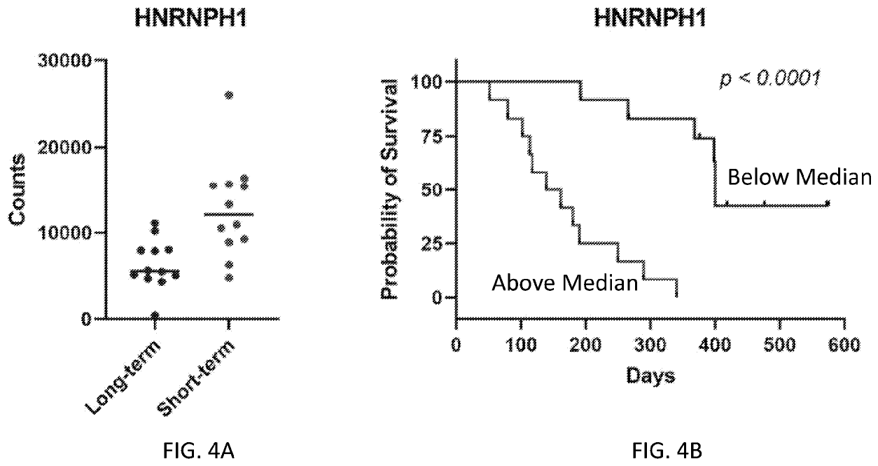

The glioblastoma patient data was further analyzed for HNRNPH1 expression levels. Glioblastoma and melanoma patients who responded better to anti-PD-1 therapy presented significant lower levels of HNRNPH1 expression. Notably, HNRNPH1 was a strong predictor of glioblastoma patient survival in response to immune checkpoint blockade (FIGS. 4A-4B and FIG. 38).

### Example 2— lncRNAs in Melanoma Cancer

### Materials and Methods

Fastq trimming. Raw pair-ended fastq files were obtained from the Gene Expression Omnibus (GEO) (accessions GSE78220, GSE121810 and GSE73721). Study metadata was obtained from GEO and from the study investigators. These files were then processed for adapter trimming and quality control using BBDuk (SourceForge). The bbduk.sh command with minlen=25, qtrim=rl, trimq=10, ktrim=r, k=25, and mink=11 trimmed low quality reads and eliminate adapter reads from the “adaptersfa” library. Contaminants were eliminated from the “phix174 μl.ref.fa.gz” library with the bbduk.sh command, using k=31 and hdist=1.

Sequence alignment. The pseudoalignment tool kallisto (Bray et al., (2016). Near-optimal probabilistic RNA-seq quantification. Nature biotechnology, 34(5), 525-527) then generated transcript counts by aligning the processed read files to a reference index built by concatenating the Ensembl coding (Homo sapiens.GRCh38.cdna.all.fa.gz) and non-coding (Homo sapiens.GRCh38.ncrna.fa.gz) libraries using bash, and then by using the kallisto index command. Counts were generated using the kallisto quant command with 100 bootstraps and 4 processors.

Expression analysis. The count data were then analyzed in RStudio. Tximport (Soneson et al., (2015). “Differential analyses for RNA-seq: transcript-level estimates improve gene-level inferences.” F1000Research, 4) and bioMart (Durinck et al., (2009). Mapping identifiers for the integration of genomic datasets with the R/Bioconductor package biomaRt. Nature protocols, 4(8), 1184-1191) were used to convert transcript abundances to gene abundances. DESeq2 (Love et al., (2014). “Moderated estimation of fold change and dispersion for RNA-seq data with DESeq2.” Genome Biology, 15, 550) was used to normalize expression counts and to obtain differentially expressed genes between cohorts. Fgsea (Korotkevich et al., (2019). “Fast gene set enrichment analysis.” bioRxiv. doi: 10.1101/060012) and the Hallmark gene sets (h.all.v7.2.symbols.gmt) (Liberzon et al., 2015 December 23;1(6):417-425. doi: 10.1016/j.cels.2015.12.004) from the MSigDB Collections (Subramanian et al., (2005). Gene set enrichment analysis: a knowledge-based approach for interpreting genome-wide expression profiles. PNAS, 102(43), 15545-15550) were used for pathway enrichment analysis, while the cell type signature gene sets database (c8.all.v7.2.symbols.gmt) (Regev et al., (2017). The Human Cell Atlas. eLife, 6, e27041) was used for cell type enrichment analysis. GSVA (Hanzelmann et al., (2013). GSVA: gene set variation analysis for microarray and RNA-seq data. BMC bioinformatics, 14, 7) was used for single-sample signature enrichment analysis. The R packages survminer and survival were used for survival analysis. Gene expression linear regression analysis was performed using the Regressit package in Excel. Heatmaps were produced with the Broad Institute's Morpheus tool, using hierarchical clustering (one minus Pearson correlation) and the 1+log 2 command on all input data.

Patient samples. In the dataset featured in Hugo et al. (Hugo et. al. (2016). Genomic and Transcriptomic Features of Response to Anti-PD-1 Therapy in Metastatic Melanoma. Cell, 165(1), 35-44), if multiple samples were collected for a single patient, all tumors from were used the transcriptomic and pathway analyses, but only one tumor sample was used in the survival analysis

In the dataset featured in Cloughesy et al. (Cloughesy et al. (2019). Neoadjuvant anti-PD-1 immunotherapy promotes a survival benefit with intratumoral and systemic immune responses in recurrent glioblastoma. Nature medicine, 25(3), 477-486), patients disqualified from the trial, patients that did not complete the trial, patients with no RNASeq data, or patients with IDH-positive tumor samples were excluded from the survival and pathway enrichment lncRNA analyses.

From the dataset featured in Zhang et al. (Zhang et al (2016). Purification and Characterization of Progenitor and Mature Human Astrocytes Reveals Transcriptional and Functional Differences with Mouse. Neuron, 89(1), 37-53), the RNASeq samples were obtained for the adult astrocytes because astrocytes are thought to be the cell of origin for glioblastoma (Jiang et al., (2012). On the origin of glioma. Upsala journal of medical sciences, 117(2), 113-121), patients with corrupted sequencing files were excluded.

Results

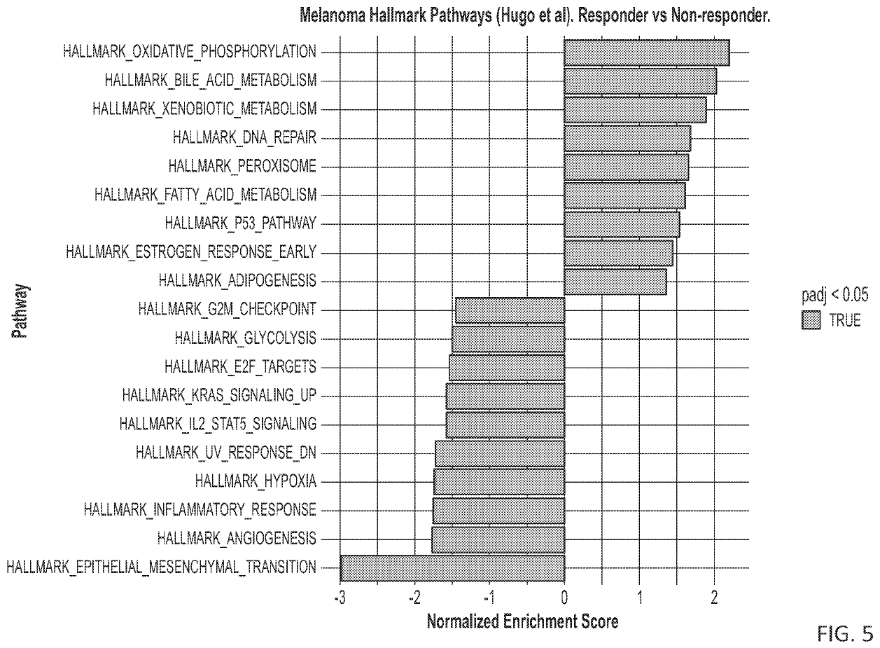

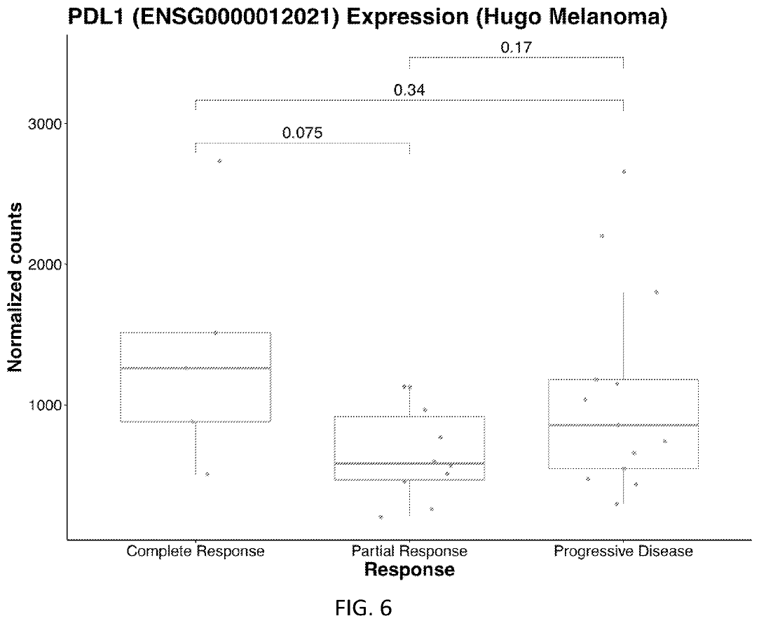

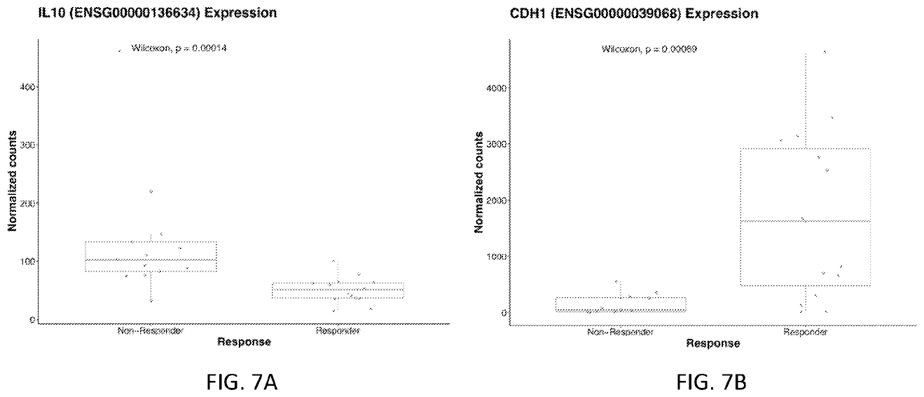

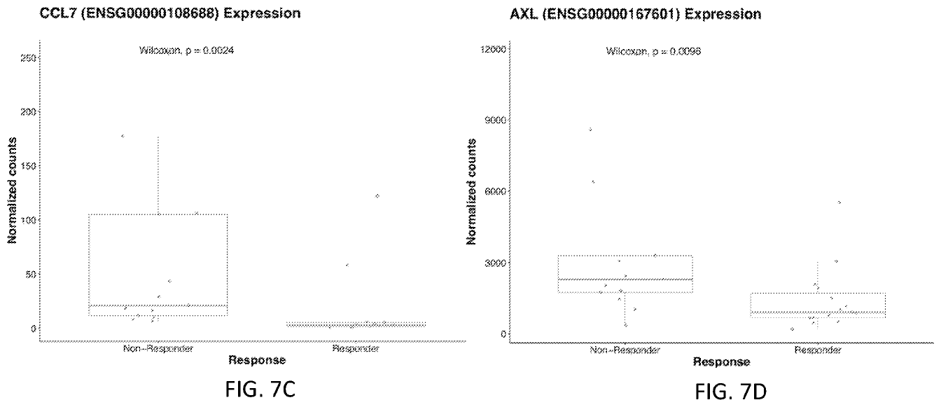

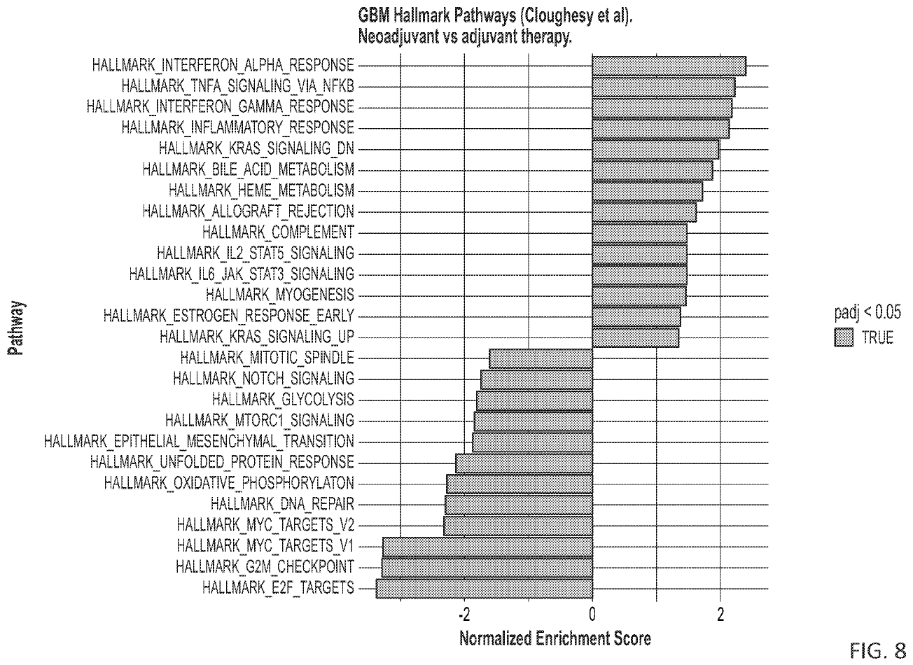

Reproduced prior transcriptomic profiling results using our analytical pipeline. After using bbduk and kallisto to generate count data for the samples from Hugo et al. (2016), Cloughesy et al. (2019), and Zhang et al. (2016), it was found that melanoma tumors that did not demonstrate either a complete or partial response to anti-PD-1 were significantly associated with pathways related to mesenchymal transition, angiogenesis, and hypoxia (FIG. 5). Next, it was found that PD-L1 expression level was not correlated to response to therapy (FIG. 6), while genes involved with tumor cell mesenchymal transition, tumor angiogenesis, and macrophage and monocyte chemotaxis were differentially expressed between the responding versus non-responding pretreatment tumors (FIGS. 7A-7D).

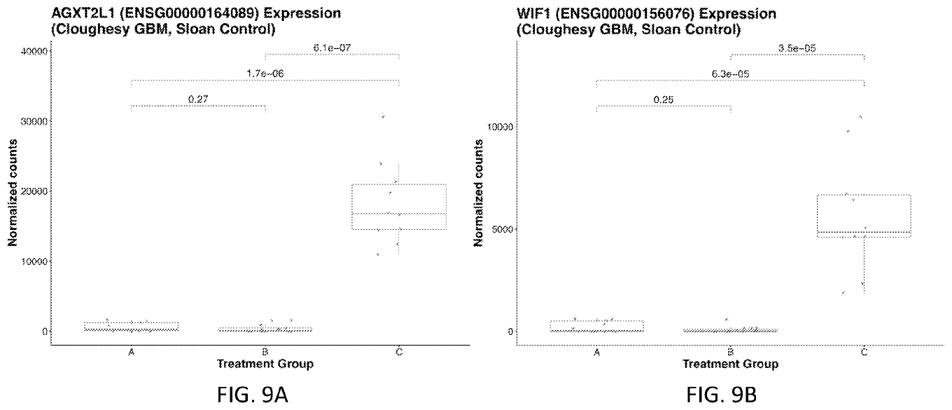

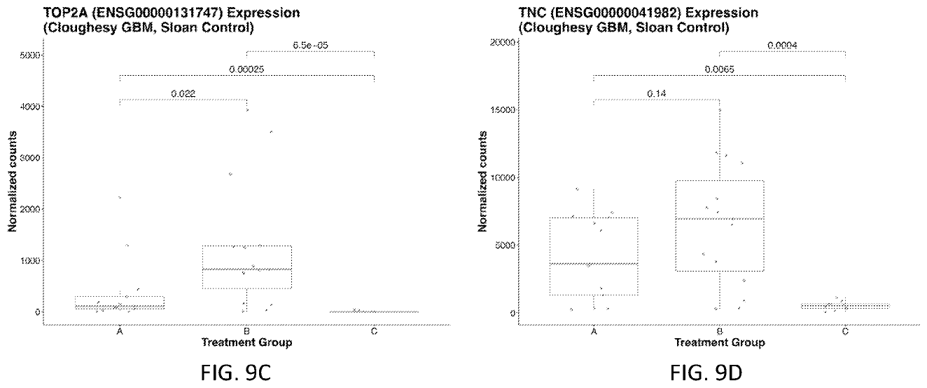

Analyzing the data from Cloughesy et al. (2019), it was found that neoadjuvant patients were enriched for the interferon-gamma response pathway, while adjuvant patients were enriched for pathways involved in cell growth (FIG. 8). Expression levels of astrocyte precursor cell marker genes and mature astrocyte marker genes were compared between healthy astrocyte samples from Zhang et al. (2016) and glioblastoma tumors from Cloughesy et al. (2019). The AGX2TL1, WIF1, TOP2A, and TNC markers were differentially expressed between healthy astrocytes and glioblastoma cells (FIGS. 9A-9D).

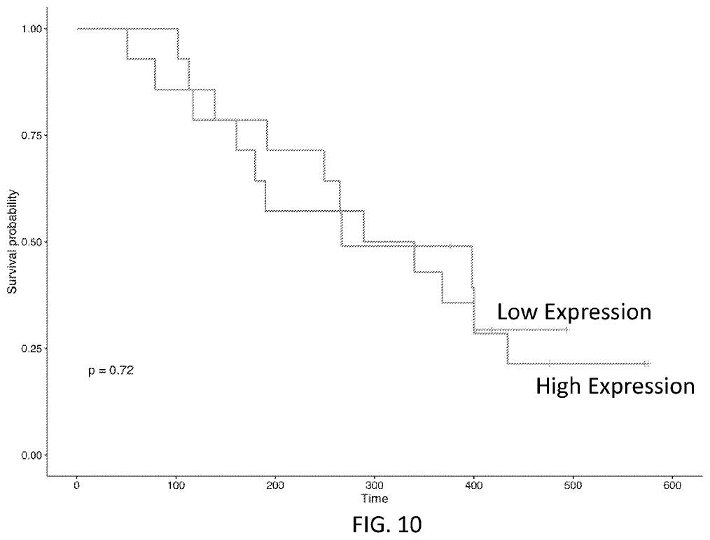

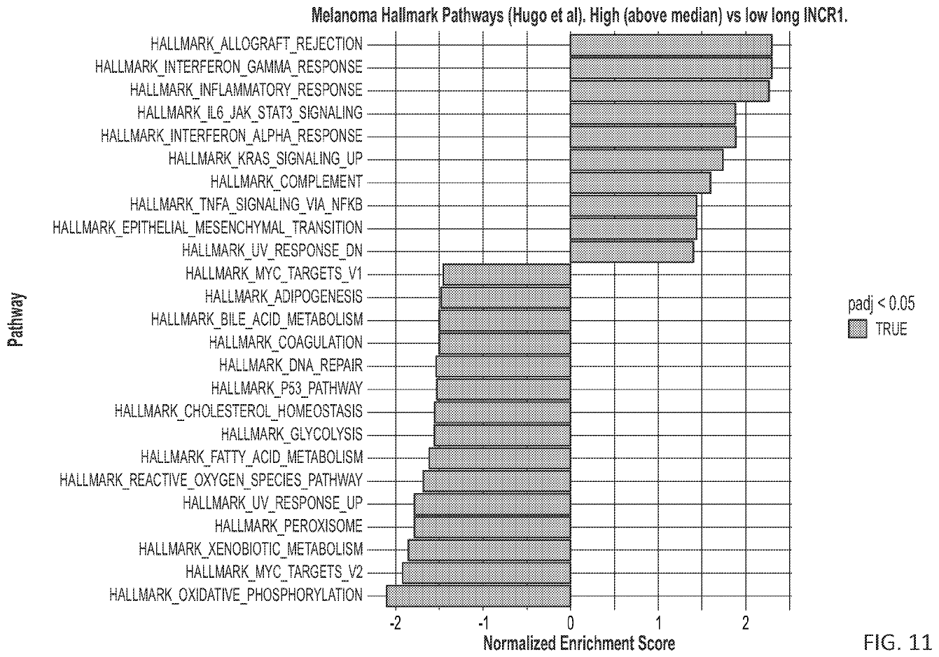

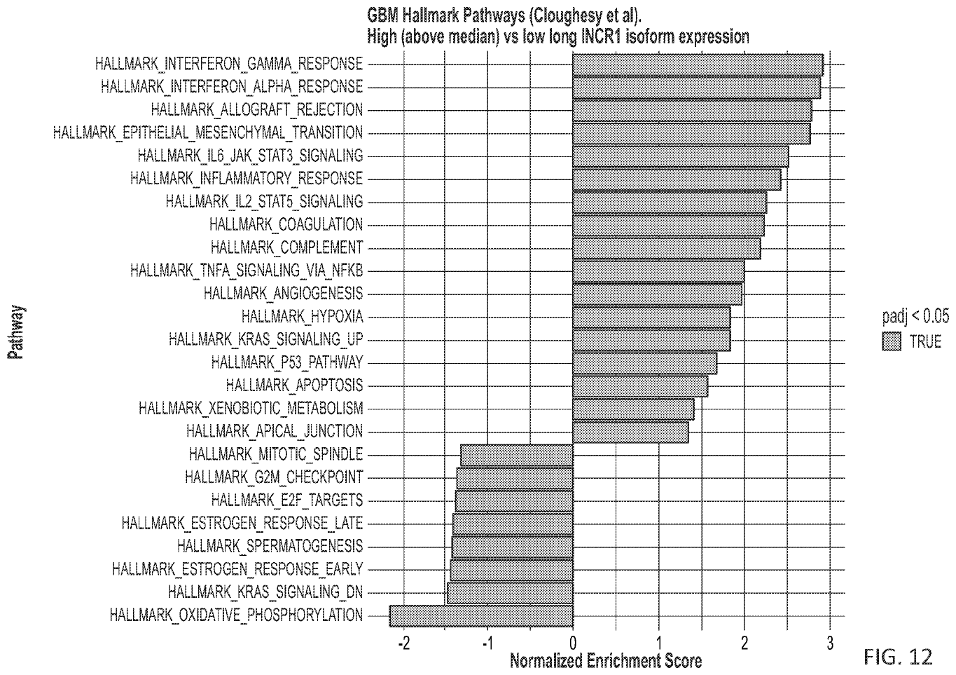

Because Mineo et al. (2020) found that the lncRNA INCR1 regulates interferon signaling in glioblastoma cells, INCR1 expression was evaluated in the melanoma and glioblastoma patient cohorts. Although INCR1 was not associated with response to immunotherapy in melanoma (FIGS. 9A-9D) nor long-term survival in glioblastoma (FIG. 10), INCR1 expression was significantly correlated with the intratumoral interferon-gamma response pathway in both melanoma and glioblastoma immunotherapy patients (FIGS. 11-12).

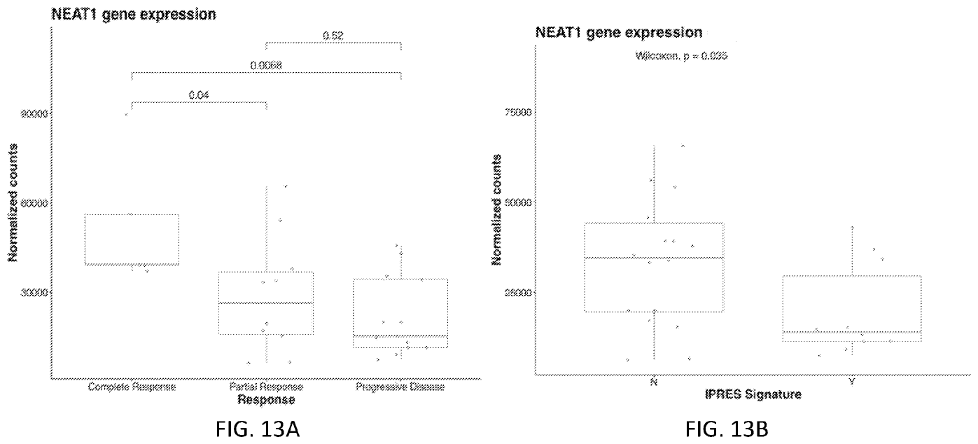

NEAT I is associated with response and survival in melanoma and glioblastoma. To identify lncRNAs with potential roles in tumor response to immune checkpoint blockade, RNAseq data was analyzed from melanoma patients treated with anti-PD-1 from the Hugo et al. (2016) cohort. 553 lncRNAs were differentially regulated in non-responders compared to complete responders to therapy (data not shown). Global lncRNA downregulation was found in patients with progressive disease compared to patients who responded to the immunotherapy. NEAT1 was identified as one of the most significantly downregulated lncRNAs in patients who did not respond to ICB therapy (FIG. 13A). NEAT1 expression was also inversely correlated with enrichment for the innate anti-PD-1 resistance (IPRES) protein-coding gene signature, (FIG. 13B), which Hugo et al. (2016) associated with non-responsiveness to anti-PD-1 and decreased likelihood of survival.

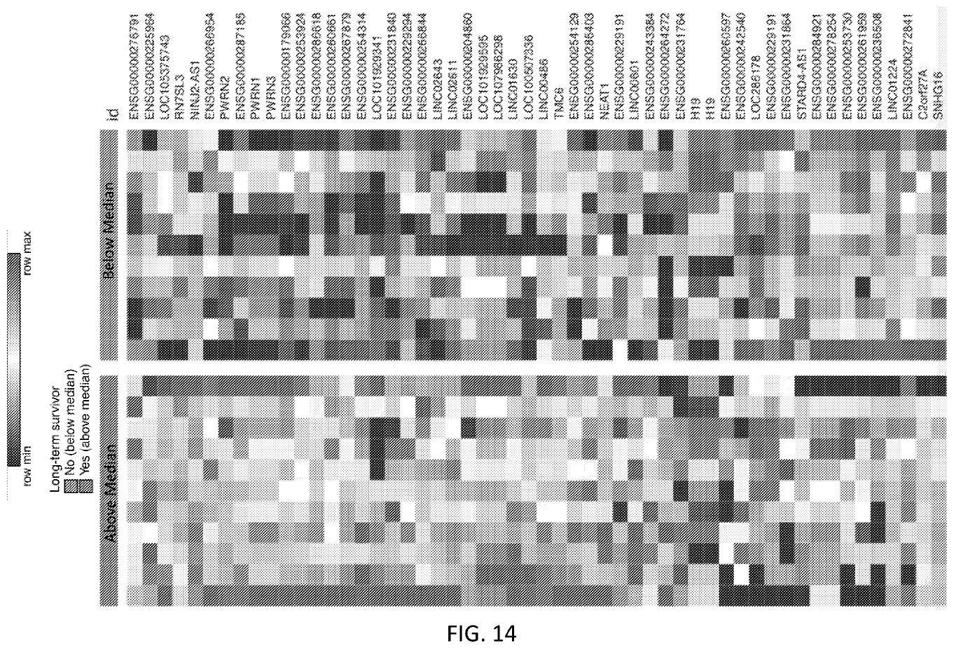

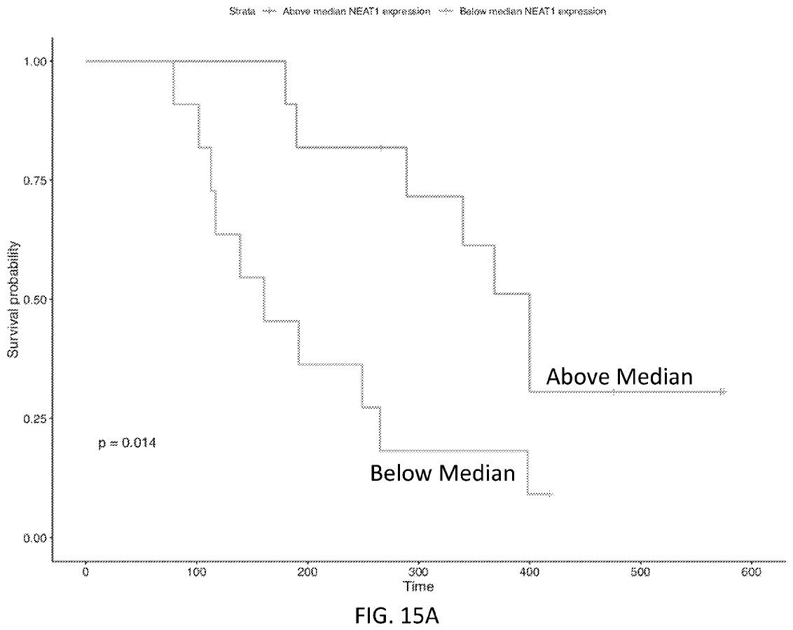

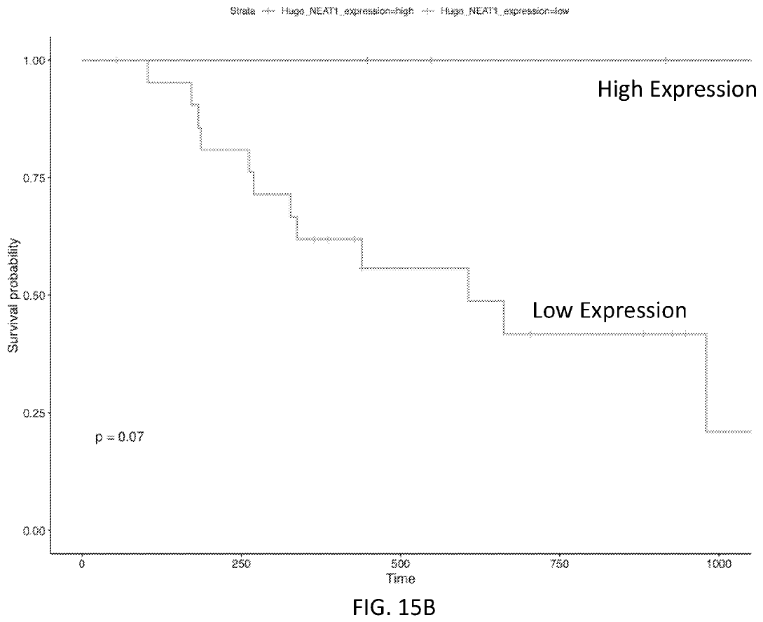

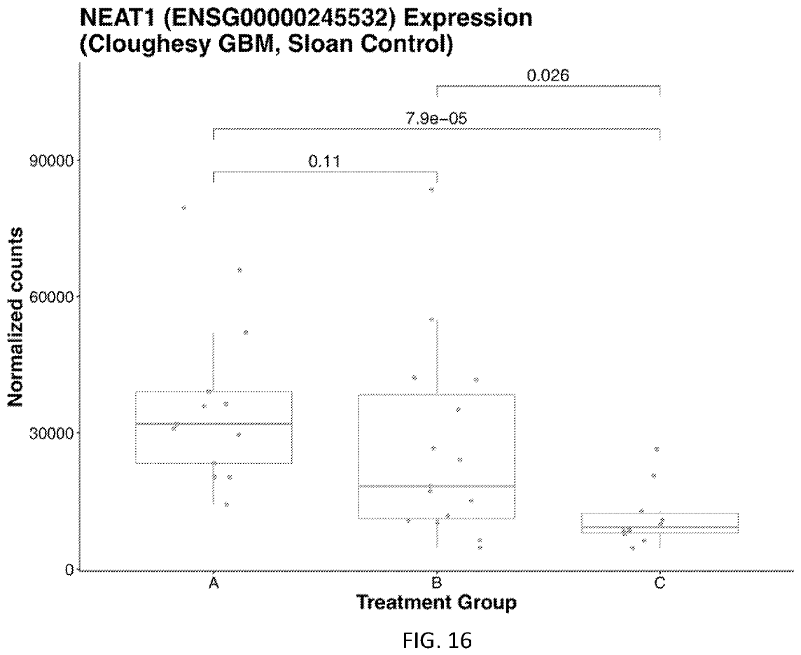

Expanding to the Cloughesy et al. (2019) study, differential expression of lncRNAs in glioblastoma was evaluated. Given glioblastoma's tendency not to respond to anti-PD-1, global lncRNA expression was analyzes on the basis of long-term survival in IDH wild type patients (n=22). 54 lncRNA genes, including NEAT1, had significantly altered expression in long-term survivors (FIG. 14, BH p value <0.01). High NEAT1 expression was associated with likelihood of survival (FIG. 15A), a finding that held when only assessing adjuvant patients, although the number of samples was limited. High NEAT1 patients also trended toward longer survival in melanoma patients (FIG. 15B). NEAT1 expression between healthy astrocytes from Zhang et al. (2016) and neoadjuvant and adjuvant glioblastoma patients was then evaluated. NEAT1 was significantly enriched in glioblastoma tumors compared to healthy astrocytes, while it trended toward higher expression in the neoadjuvant glioblastoma patient cohort (FIG. 16).

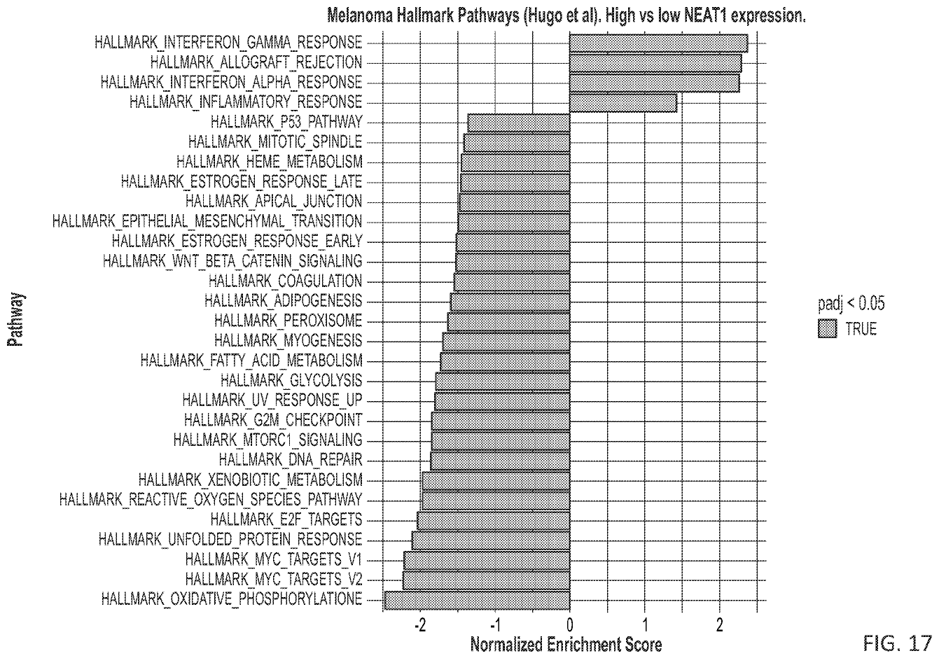

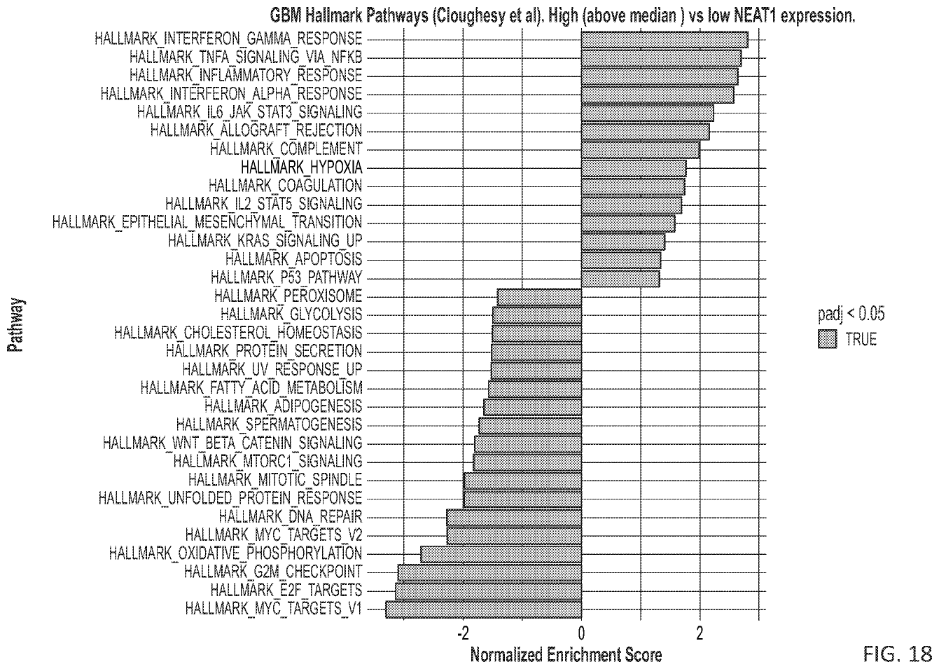

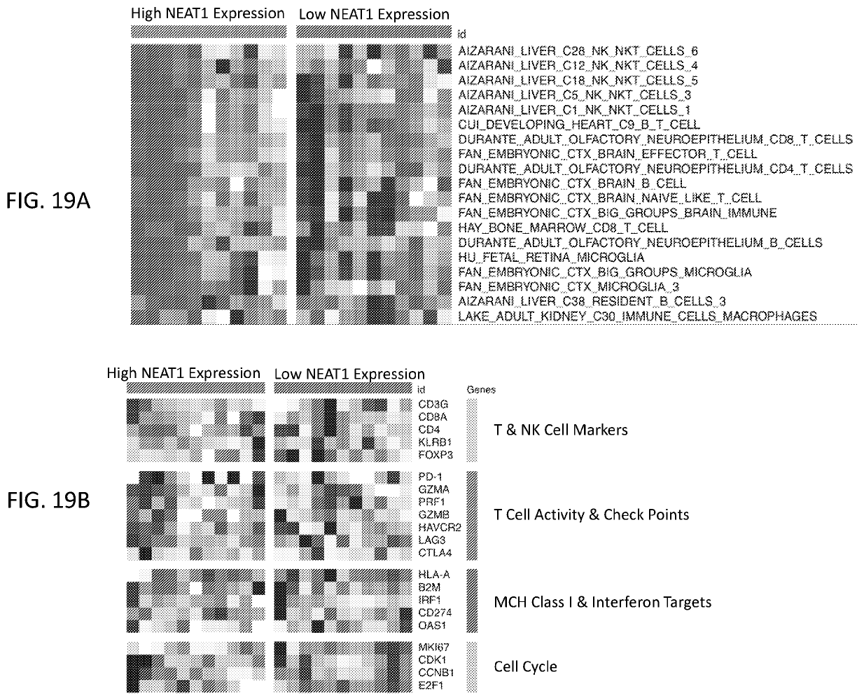

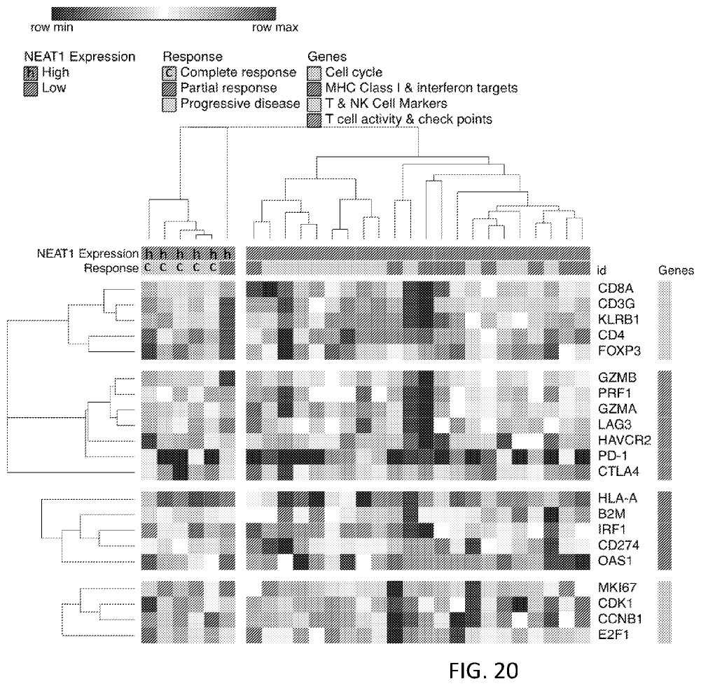

Next, pathway and cell signature enrichment analyses were performed to identify possible biological roles of NEAT1. In both melanoma and GBM patients, NEAT1 expression strongly correlated with the interferon-gamma response pathway (FIG. 17), while low NEAT1 expression was associated with cellular division and metabolic processes (FIG. 18). It was also found that GBM tumors with high NEAT1 expression were enriched for immune cell signatures (FIG. 19A). The expression of immune and cell cycle markers studied by Cloughesy et al. (2019) was also altered between high vs low NEAT1 tumors (FIG. 19B). Similar findings were observed in melanoma (FIG. 20).

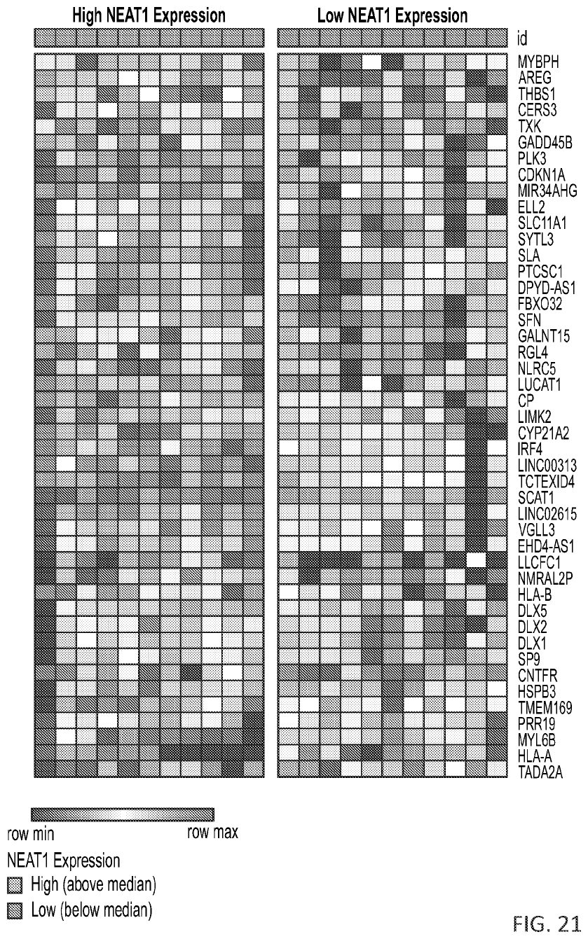

Differential gene expression on the basis of NEAT1 expression was assessed. A variety of genes were differentially regulated between high vs low NEAT1 GBM tumors (FIG. 21, BH p value <0.01), including TXK, which contributes to IFN-gamma transcription (Takeba et al., 2002), and NLRC5, which regulates MHC class I-dependent immune responses (Kobayashi et al., 2012). Interestingly, NEAT1-high tumors were upregulated for the human leukocyte antigen (HLA)-B, but downregulated for HLA-A. Therefore, the data indicate that NEAT1 may play an important role in regulating the tumor immune response.

### Example 3—Post-Transcriptional Mechanisms of Tumor Interferon Signaling Regulation

To determine post-transcriptional mechanisms of tumor interferon signaling regulation, the binding partners of INCR1 were determined because lncRNA INCR1 transcribed from the PD-L1 locus regulates interferon (See, for example, Mineo et al. Mol Cell. 2020. 78: 6, 1207-1223.e8).

Methods

### Experimental Model and Subject Details

Cell Lines. Patient-derived primary GBM cells (PDGCLs, BT cell lines) were generated as previously described (Stevens et al., 2016). U251 cells were obtained from the NCI-DTP. U1242 cells were obtained from James Van Brocklyn (Ohio State University). A375 cells were obtained from Frank Stephen Hodi (Dana-Farber Cancer Institute). BT cell lines were cultured as neurospheres in stem cell conditions using Neurobasal (Thermo Fisher Scientific) supplemented with Glutamine (Thermo Fisher Scientific), B27 (Thermo Fisher Scientific), 20 ng/ml epidermal growth factor (EGF) and fibroblast growth factor (FGF)-2 (PrepoTech). U251, U1242, A375 cells were cultured in DMEM(Thermo Fisher Scientific) supplemented with 10% fetal bovine serum (FBS, Sigma-Aldrich) and 100 U/ml penicillin-streptomycin (Thermo Fisher Scientific). All cell lines were maintained in humidified 5% CO2 incubator at 37_C.

### Method Details

Cell culture and transfection. Unless otherwise specified, IFNg (PeproTech) stimulation was performed at 100 U/ml IFNg for a period of 24 h. IFNb and TNFa were obtained from PeproTech. Stable U251, A375 and MDA-MB-231 knockdown were obtained by transducing cells with shRNA 1 (clone: CS-SH128T-3-LVRU6GP; target sequence: GCCATTGCAGGAAATATAAGA (SEQ ID NO:94), GeneCopoeia) and shRNA 2 (clone: CSSH128T-6-LVRU6GP; target sequence: CAGCTCTCAATTCTGTGAAACTCAA (SEQ ID NO:95), GeneCopoeia). LNA GapmeRs (Exiqon) knockdown experiments were performed transfecting BT cells with 50 nM of GapmeR (TTACATGATGACCTTT, SEQ ID NO:96) using Lipofectamine 2000 (Thermo Fisher Scientific). Stable U251-EGFRvIII were obtained by infecting cells with pLVIRES-mCherry-EGFRvIII vector. HNRNPH1 knockdown was performed transfecting 50 pmol/well of Duplex siRNAs (hs.Ri.HNRNPH1.13.1 and hs.Ri.HNRNPH1.13.2, Integrated DNA Technologies) for 6 well plate using Lipofectamine RNAiMAX (Thermo Fisher Scientific). HNRNPH1 binding site blocking experiments were performed transfecting cells with 100 nM of fully 20-O-Methoxyethyl (20-MOE) and phosphorothioate bond modified antisense oligonucleotide control (ASO NC, GCGACTATACGCGCAATATG, SEQ ID NO:97) or targeting HNRNPH1 binding site on the INCR1 gene (ASO H1B, CTCCAGCTCCCCCCGGCAAC, SEQ ID NO:98) (Integrated DNA Technologies).

Quantitative Real-Time PCR analysis. Total RNA from cell lines and patients' tissues was extracted using TRIzol (Thermo Fisher Scientific). Nuclear/cytoplasmic fractionation was performed as previously described (Mineo et al., 2016). RNA was reverse-transcribed using iScript cDNA Synthesis Kit (BioRad) and quantitative real-time PCR was performed using SYBR Green Master Mix (Applied Biosystem). 18S expression levels were used as control. For copy number analysis, absolute quantification of INCR1 and PD-L1 RNA was performed using the standard-curve method. The primers used in this study are listed in Table 2. Immunoblot analysis and antibodies Immunoblotting was performed as previously described (Mineo et al., 2016). The following antibodies were used: anti-PD-L1, anti-IDO, anti-JAK2, anti-STAT1, anti-phospho-STAT1 and anti-b-Actin (13684, 86630, 3230, 9172, 9167 and 3700, respectively, Cell Signaling Technology); anti-hnRNP-H (A300-511A, Bethyl Laboratories).

T cell cytotoxicity assay. 750 RFP positive control or HNRNPH1-knockdown tumor cells were seeded in a round bottom low-attachment 96 well plate. Cells were allowed to form tumorspheres for 72h. After tumorspheres were formed, two thousand CAR T cells stimulated with Dynabeads Human T-Activator CD3/CD28 and 10 ng/ml interleukin-2 were added. Tumorspheres and CAR T cells were co-cultured for 18 to 48 h and changes in RFP intensity were measured using ImageJ.

In vitro T cells transduction. Generation of T cells expressing chimeric antigen receptor (CAR) against EGFRvIII is described in Khalsa J K et al., manuscript under preparation. In brief, the EGFRvIII CAR was constructed as described previously (Johnson et al., 2015) using the self-inactivating lentiviral transfer vector pRRL.PPT.EFS bearing an IRS-GFP cassette and packaged as described previously (Shah et al., 2008). pRRL.PPT.EFS-GFP vector served as control. T cells were isolated from PBMCs by EasySep Human T cell isolation kit (Stem Cell Technology). Isolated T cells were counted and cultured at 1:1 ratio with Dynabeads human T activator CD3/CD28 (Thermo Fisher Scientific) in X-vivol5 medium supplemented with 30U/ml IL-2. Next day, 1.5 million T cells/ml were transduced with EGFRvIII-CAR or control lentivirus at MOI 10 and 6 mg/ml polybrene in 6 well plates. Medium was replaced next morning and GFP expression was checked 48 hours post-infection. Before injecting T cells in mice, the ability of EGFRvIII-specific CAR T cells to kill target cells was tested in vitro by 3D T cell cytotoxicity assay.

UV-cross/ink RNA immunoprecipitation. UV-crosslink RNA immunoprecipitation assay was performed as previously described with some modifications (Mineo et al., 2016). Briefly, cells were UV irradiated at 400 mJ/cm2 and nuclear extracts were prepared by incubating cells in RLN Buffer (50 mM Tris, 1.5 mM MgCl2, 150 mM NaCl, 0.5% NP-40, protease inhibitors) for 5 min. Nuclei were pelleted by centrifuging at 1,450×g for 2 min and lysed for 10 min in CLIP Buffer (50 mM Tris, 150 mM NaCl, 1% NP-40, 0.1% Sodium Deoxycholate, phosphatase and protease inhibitors, 100 U/ml RNase inhibitor [New England BioLabs]). Samples were sonicated with microtip, 5 W power (25% duty) for 60 s total in pulses of 1 s on followed by 3 s off. DNA was digested incubating samples for 15 min at 37_C in 1x DNase salt solution (2.5 mM MgCl2, 0.5mMCaCl2) with 30 U TurboDNase. EDTA was added to the samples to a final concentration of 4mMand samples centrifuged at 16,000×g for 10 min. Nuclear extracts were precleared with Protein A/G Plus Agarose beads (Thermo Fisher Scientific) and incubated with primary antibody (anti-hnRNP-H) or rabbit IgG control (Bethyl Laboratories) overnight at 4_C. Protein/RNA complexes were precipitated using Protein A/G Plus Agarose beads (Thermo Fisher Scientific). Beads were washed and incubated with Proteinase K (Thermo Fisher Scientific) and RNA was extracted using TRIzol.

Enhanced CLIP (eCLIP). A375 cells were stimulated with IFNg for 6 h and UV irradiated at 400 mJ/cm2. eCLIP was performed by EclipseBiolnnovations as previously described (Van Nostrand et al., 2016). Expression and purification of HNRNPH1 HNRNPH1 isoform A was cloned in pET21-His-Smt3 and protein expressed by transformation of Rosetta-2 (DE3) pLys(S) E. coli (EMD Millipore). Cells were lysed in 20 mL of 50 mM Tris [pH 8.0], 300 mM KCl, 0.02% NP-40, 10 mM Imidazol, 10% Glycerol, 0.1 mM EDTA, 0.1 mM DTT, 0.1 mg/ml lysozyme. Re-suspended cells were incubated on ice for 20 min. Cells were further disrupted and DNA was sheared by sonication (3, 20 s bursts with 20 s rests). Insoluble material was pelleted by centrifugation (30 min at 20,000×g at 4_C). Soluble material was decanted. Insoluble pellet was resolubilized in 50 mM Tris [pH 8.0], 300 mM KCl, 0.02% NP-40, 10 mM Imidazol, 10% Glycerol, 0.1 mM EDTA 0.1 mM DTT, 6 M Urea followed by sonication. Remaining insoluble material was pelleted by centrifugation (30 min at 20,000×g at 4_C). Soluble material was decanted to new tube. Expression was analyzed by Coomassie staining. 1 mL of TALON resin (Clontech) was equilibrated in respective lysis buffers and added to lysates. Beads and lysates were tumbled at 4_C for 2 h. Beads were washed 2 times in 50 mL lysis buffer and loaded onto column. Column was washed with 10 mL of lysis buffer and then eluted in Lysis buffer containing 300 mM Imidazole. 10 fractions of 1.5 mL were collected and analyzed by Coomassie staining. Protein purified under denaturing conditions was dialyzed against 50 mM Tris [pH 8.0], 300 mM KCl, 0.02% NP-40, 10 mM Imidazole, 10% Glycerol, 0.1 mM EDTA 0.1 mM DTT, 4 M Urea overnight. The following day, protein was dialyzed against 50 mM Tris [pH 8.0], 300 mM KCl, 0.02% NP-40, 10 mM Imidazole, 10% Glycerol, 0.1 mM EDTA 0.1 mM DTT, 2 M Urea for 4 h and then 50 mM Tris pH 8.0, 300 mM KCl, 0.02% NP-40, 10 mM Imidazole, 10% Glycerol, 0.1 mM EDTA 0.1 mM DTT, 1 M Urea for 4 h. Finally, the protein was dialyzed against 50 mM Tris [pH 8.0], 300 mM KCl, 0.02% NP-40, 10% Glycerol, 0.1 mM EDTA 0.1 mM DTT, 0 M Urea overnight. Dialyzed protein was clarified by centrifugation (30 min at 20,000×g at 4_C). Purity of protein was analyzed by Coomassie staining.

Biotinylated RNA pulldown assay. Genomic DNA was extracted from cell cultures to generate amplicons corresponding the 50 and 30 ends of the INCR1 intron 1. PCR products were cloned in pCR2.1-TOPO (Thermo Fisher Scientific) to generate pCR2.1-INCR1 Intron 1 (50 half) and pCR2.1-INCR1 Intron 1 (30 half). PCR products from pCR2.1-INCR1 Intron 1 (30 half) were generated to add HindIII linkers to the 30 end and this fragment was cloned between SpeI and HindIII in pCR2.1-INCR1 Intronl (50 half) to generate a pCR2.1-INCR1 minigene. In vitro transcripts of biotinylated RNA were generated by PCR and numbered fragment 1-7 in a 50 to 30 direction. Each fragment allowed for transcription of a 300 nucleotide RNA, each with a 50 nucleotide overlap to the adjacent fragment. T7 promoter sequence was added by PCR. In vitro transcription reactions were performed using T7 HiScribe (New England Biolabs) according to manufacturer's instructions, except the final concentration of UTP was reduced to 7.5 mM and instead supplemented with 2.5 mM Biotin-16-UTP (Sigma Aldrich). Transcribed RNAs were extracted by acidic phenol chloroform extraction (Thermo Fisher Scientific) and precipitated with ammonium acetate. Unincorporated nucleotides from resuspended RNAs were removed by gel filtration chromatography through Illustra Microspin G-25 columns (GE Healthcare). Concentrations of each RNA was brought to 4 mM with DEPC-treated H₂O (Thermo Fisher Scientific). 1 ml of 4 mM biotinylated in vitro transcribed RNA was added to cell lysates and protein complexes allowed to assemble for 2 h at 4_C. After incubation, 10 ml of streptavidin-agarose (Thermo Fisher Scientific) were added and tumbled for an additional hour. Beads were washed 4 times with lysis buffer and complexes were eluted with 2x SDS loading buffer. Eluted proteins were resolved on 4-20% gradient gel (Bio-Rad) and assayed by western blotting. For RNA pulldown assays with blocking oligos, prior to preforming pulldown assay, 4 pmol of biotinylated RNA was incubated with indicated amount of blocking oligo in 20 ml of binding buffer (10mMTris [pH 7.9], 50mMNaCl, 10mMiMgCl2 1mMDTT). RNA/oligo mixture was incubated at 90_C for 5 minutes and allowed to cool to room temperature for 30 minutes to facilitate annealing. The primers used in this assay are listed in Table 2.

RNA electrophoretic mobility shift assay. Synthetic RNA was obtained from IDT and radiolabeled with g-32P ATP (6000 Ci/mmol, Perkin-Elmer) using T4 Polynucleotide Kinase (New England Biolabs). Unincorporated nucleotides were removed by gel filtration chromatography through Illustra Microspin G-25 columns (GE Healthcare). RNA/protein complexes were allowed to form at room temperature by adding indicated amount of protein to 1 pmol of radiolabeled RNA in 20 ml reaction containing 50 mM Tris [pH 8.0], 300 mM KCl, 0.02% NP-40, 10% Glycerol, 0.5 mg/ml Heparin for 10 minutes. Complexes were loaded onto native polyacrylamide gels and ran for 3 h at 150 V. Gels were dried and visualized by autoradiography. The synthetic RNAs used in this assay are listed in Table 2.

Microscale thermophoresis. MicroScale Thermophoresis experiments were performed according to the NanoTemper technologies protocol in a Monolith NT.115Pico (red/blue) instrument (NanoTemper Technologies). Serial dilutions of HNRNPH1 were done using a buffer containing 50 mM Tris [pH 8.0], 300 mM KCl, 0.02% NP-40, 10% Glycerol, and 0.5 mg/ml Heparin. RNA oligos were labeled with a FAM moiety at their 30 ends (IDT). The RNA concentration was kept constant at 20 nM throughout the experiments. The RNA-protein mixture was incubated at room temperature for 15 mins before running into the MST instrument. The experiments were performed using 40% and 60% MST power and between 20%-80% LED power at 22_C. The MST traces were recorded using the standard parameters: 5 s MST power off, 30 s medium MST power on and 5 s MST power off. The reported measurement values are the combination of two effects: the fast, local environment dependent responses of the fluorophore to the temperature jump and the slower diffusive thermophoresis fluorescence changes. The data presented here are the average of 3 independent experiments. Average normalized fluorescence (%) was plotted against HNRNPH1 concentration to determine the binding constant (Kd). Ligand depletion model with one binding site was used (Using GraphPad Prism 8) to fit the binding which follows the following model: Y=Bmax*X/(Kd+X). The synthetic RNAs used in this assay are listed in Table 2.

QUANTIFICATION AND STATISTICAL ANALYSIS. Graphs were generated and statistical analysis was performed using Prism (GraphPad). Statistical details of experiments, including number of experiments, statistical test and statistical significance (p value) are reported in the figure legends. Independent experiments were performed to define the reproducibility of the results.

Results

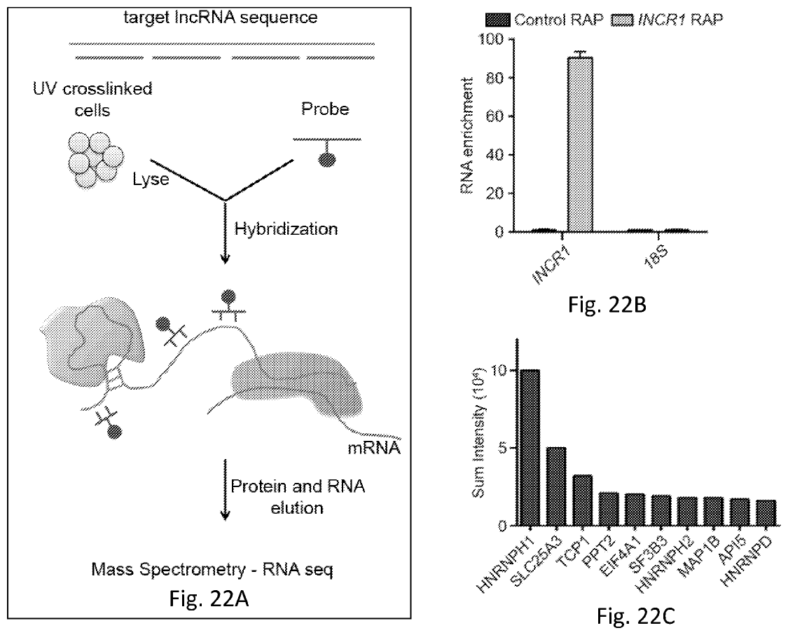

HNRNPH1 was determined to be a binding partner of INCR1 through RNA antisense purification (RAP). Cells were UV crosslinked and a lncRNA INCR1 probe was used to pull down endogenous INCR1. Then protein and RNA were eluted, followed by analysis of the protein portion with mass spectrometry and the RNA portion with RNA-seq (FIGS. 22A-22C). HNRNPH1 was the highest enriched protein when INCR1 was used as the RNA bait.

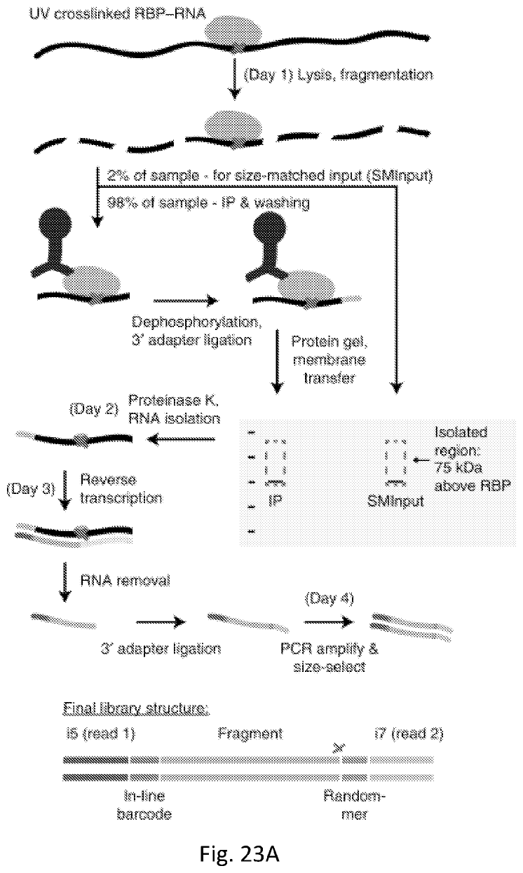

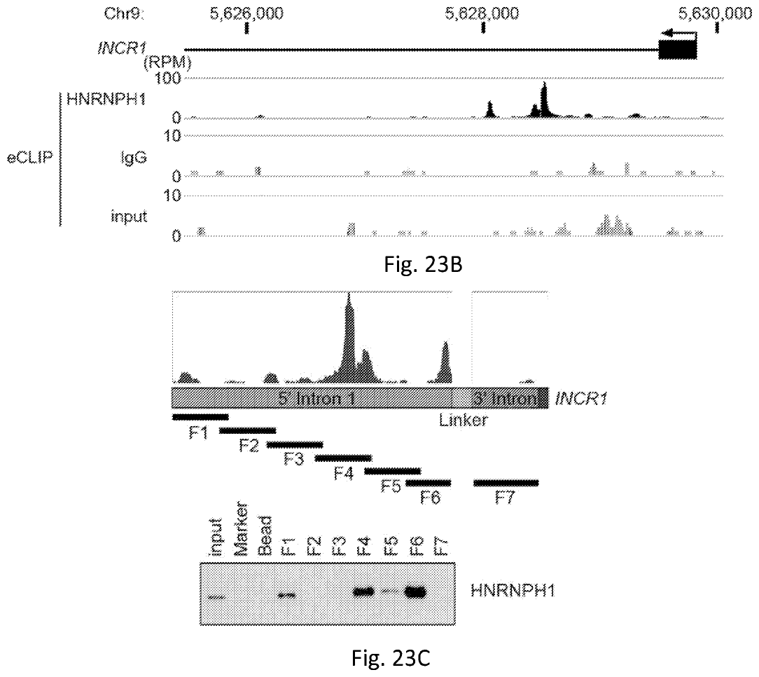

HNRNPH1 was determined to bind INCR1 in the proximal intron with HNRNPH1-RNA crosslinking followed by RNA sequencing (FIGS. 23A-23C).

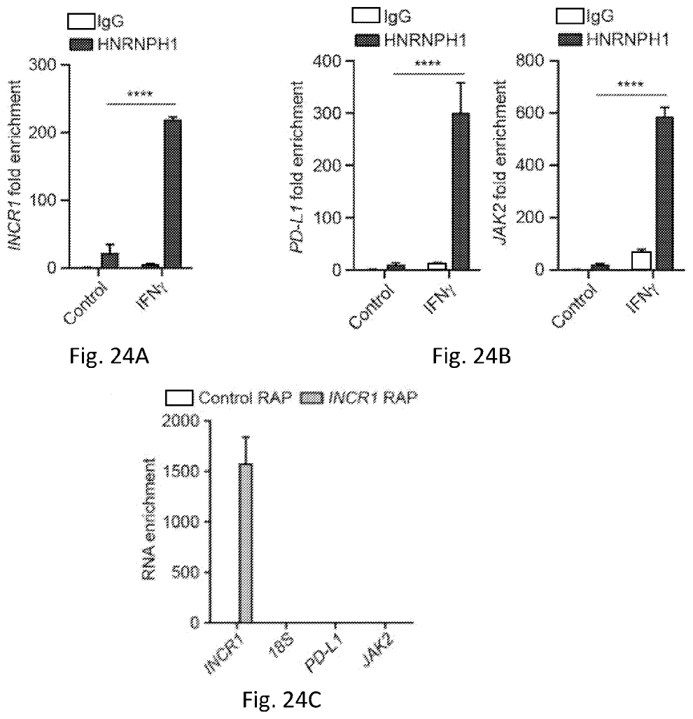

HNRNPH1 was shown to bind INCR1, PD-L1 and JAK2 with RNA immunoprecipitation (FIGS. 24A-24C). INCR1 was determined not to directly interact with PD-L1 and JAK2 with RAP.

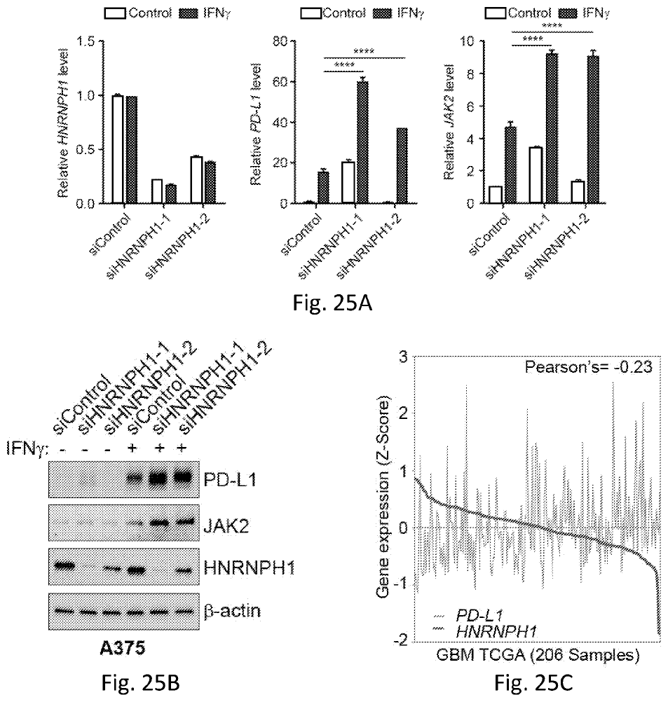

HNRNPH1 was shown to be a negative regulator of PD-L1 and JAK2 expression (FIGS. 25A-25C).

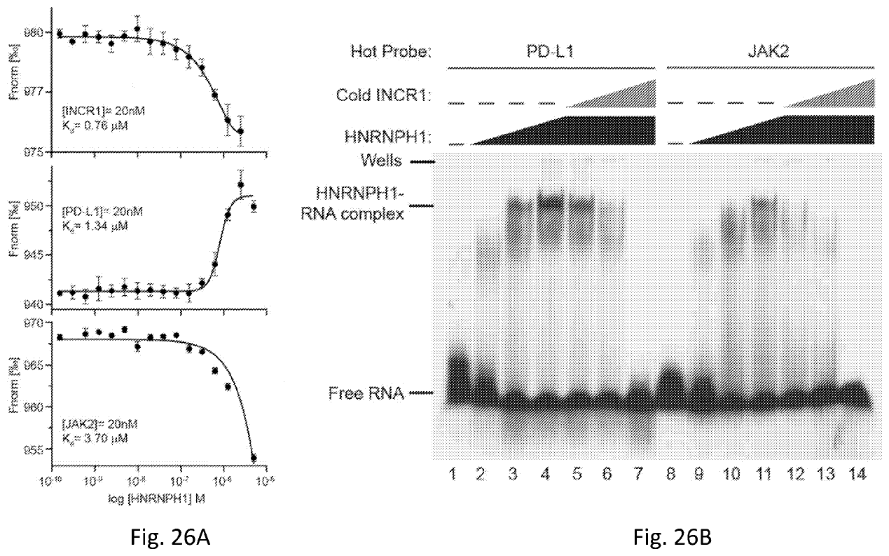

INCR1 was shown to interfere with HNRNPH1 binding to PD-L1 and JAK2 (FIGS. 26A-26B).

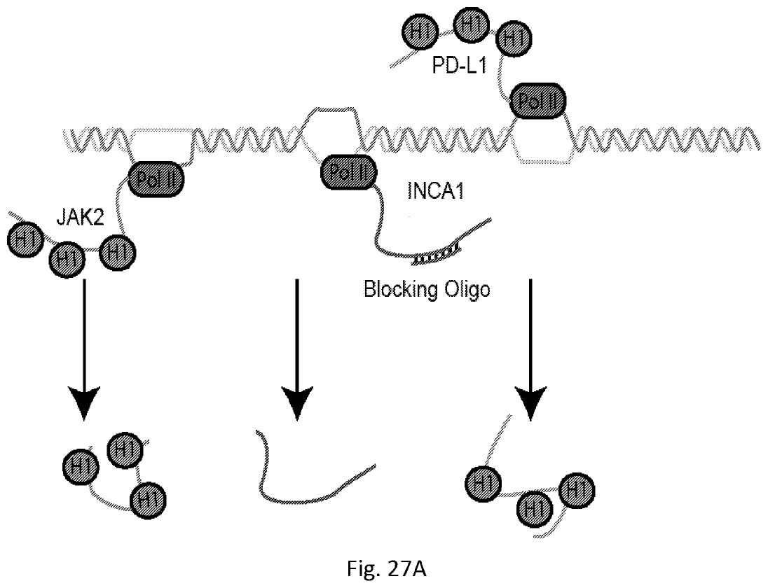

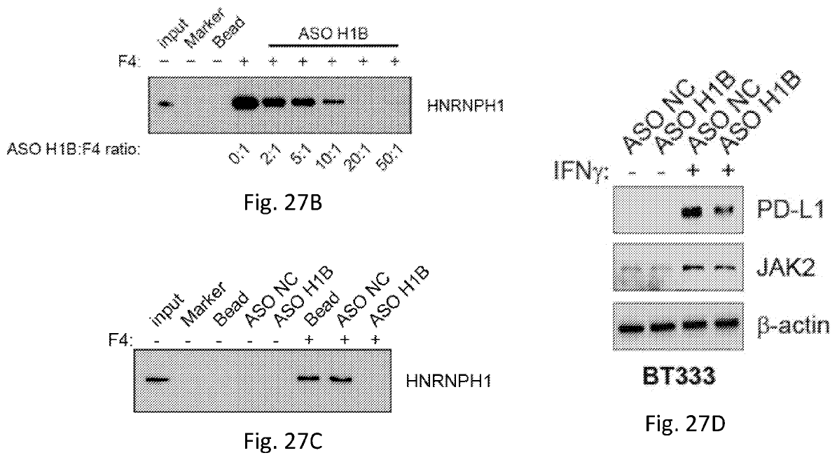

Blocking HNRNPH1 binding to INCR1 resulted in reduced PD-L1 and JAK2 expression (FIGS. 27A-27D), indicating that INCR1 bound HNRNPH1 to allow PD-L1 and JAK2 expression.

An RNA-sequencing library was generated to determine the proteins that HNRNPH1 interacted with (FIG. 23A).

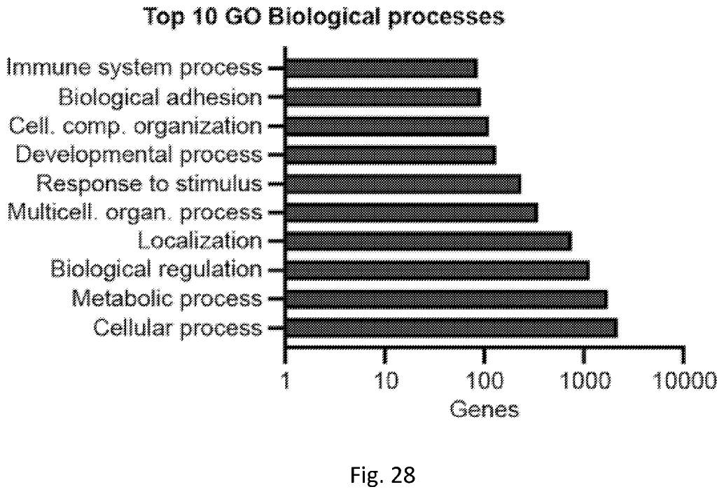

HNRNPH1 was shown to bind genes involved in immune function (FIG. 28).

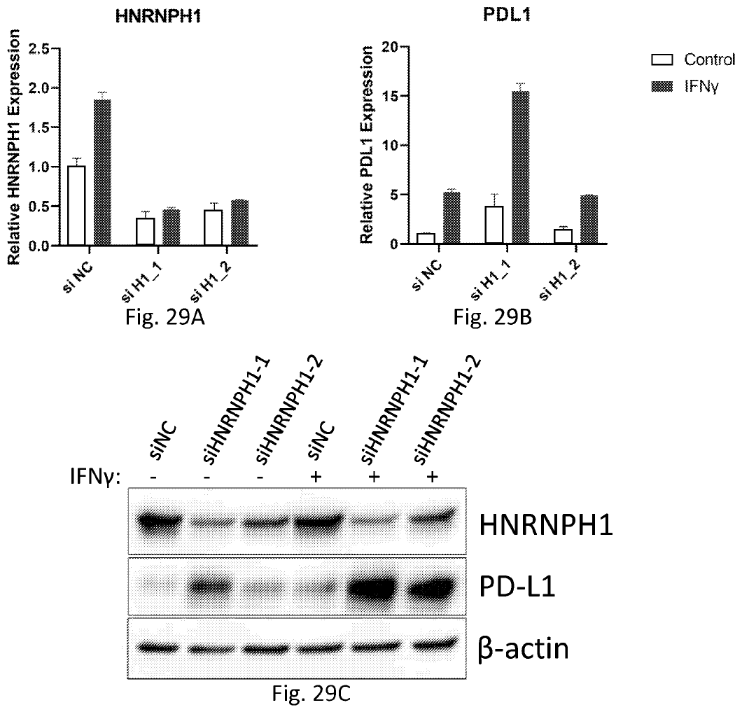

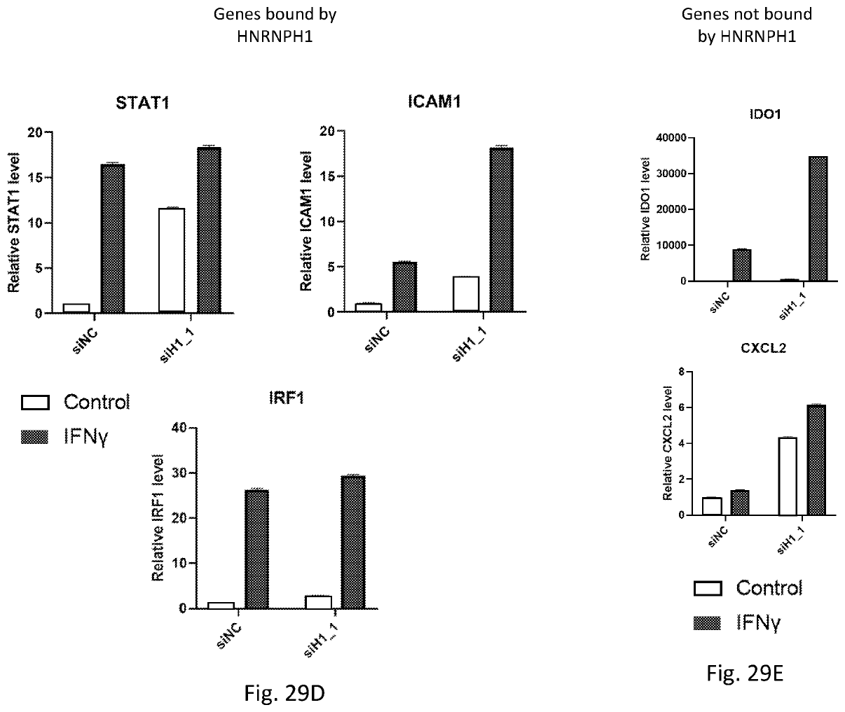

HNRNPH1 was shown to be a negative regulator of interferon-stimulated genes (FIGS. 29A-29E).

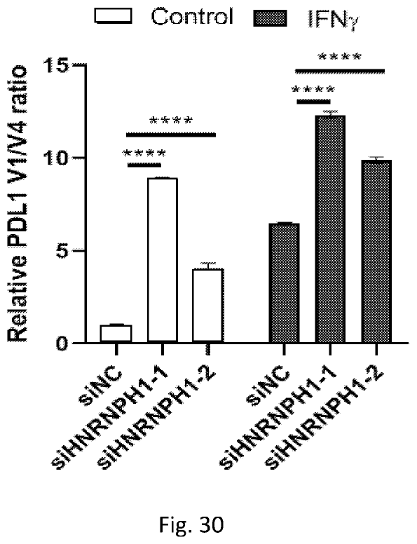

Silencing RNA experiments showed that HNRNPH1 regulated the expression of PD-L1 variant 1 (FIG. 30).

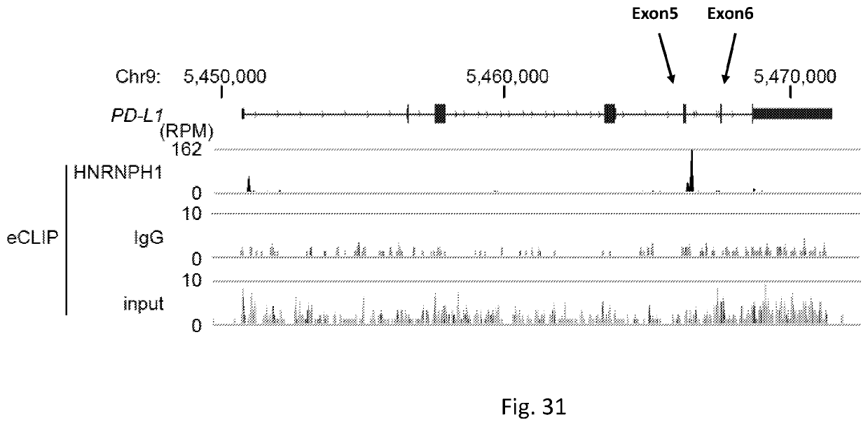

HNRNPH1 was shown to bind to intron 5 of PD-L1 (FIG. 31).

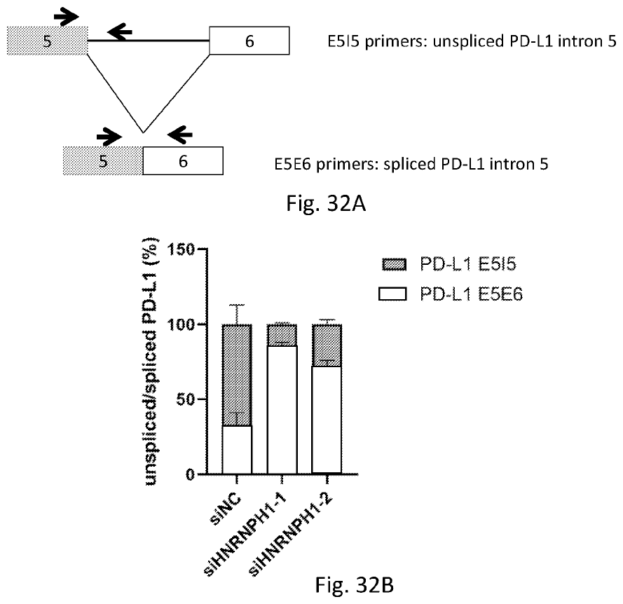

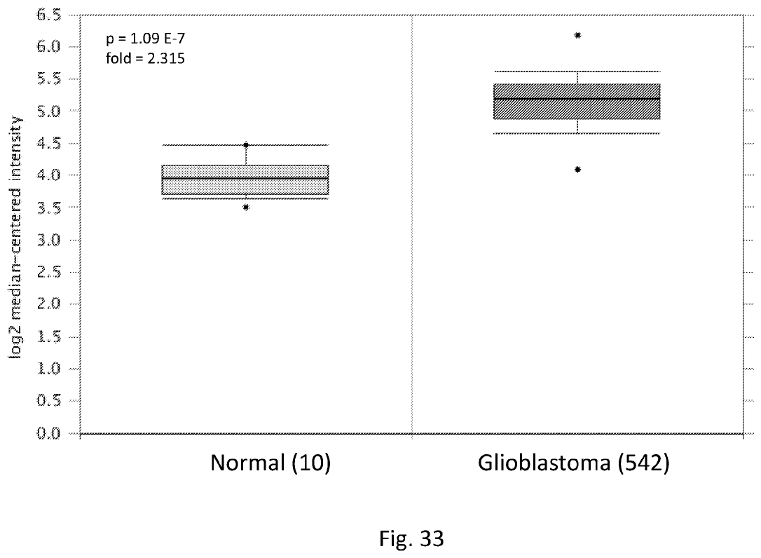

HNRNPH1 regulated PD-L1 splicing as two different siRNA targeting HNRNPH1 affected the percentage of spliced (white bars) and unspliced (grey bars) PD-L1 (FIGS. 32A-32B).

HNRNPH1 was upregulated 2.315 fold in glioblastoma compared to normal (FIG. 33), indicating that HNRNPH1 is overexpressed in glioblastoma tumors.

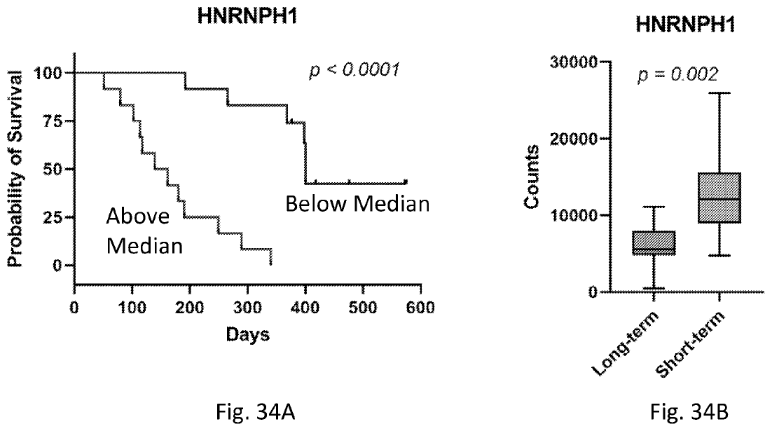

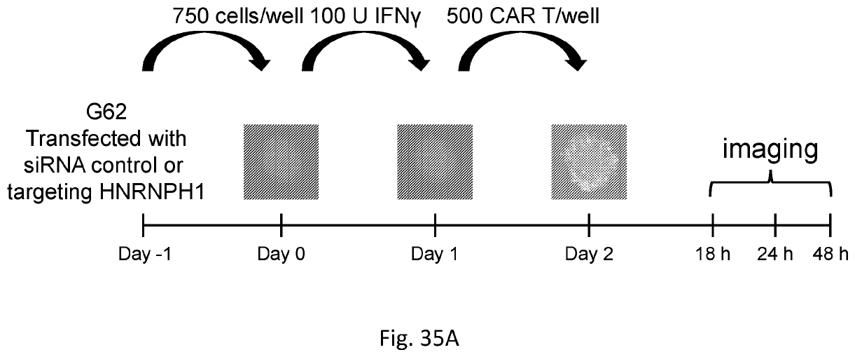

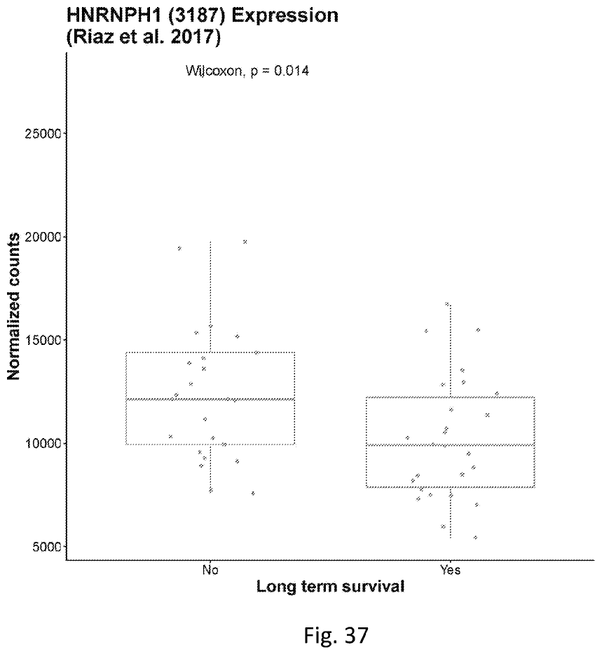

HNRNPH1 expression level predicts patient response to immune checkpoint therapy in glioblastoma (FIG. 34A) and showed that subjects with low HNRNPH1 levels responded better to immune checkpoint inhibitors than subjects with high HNRNPH1 levels. Moreover, long term glioblastoma patient survivor had lower HNRNPH1 expression compared to short term survivor (FIG. 34B) Similarly, long term survival of patients with melanoma showed lower HNRNPH1 levels compared to short term survivor (FIG. 37). Data was obtained from Raiz et al. 2017. Cell, 171:4(2), 934-949.e16, and analyzed separately.

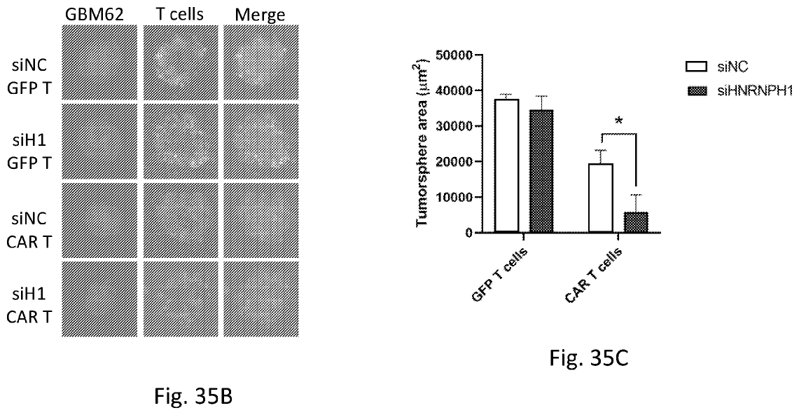

A 3D CAR T cell cytotoxicity assay showed that silencing HNRNPH1 improves CAR T cell activity (FIGS. 35A-35C). Briefly, G62 cells were transfected with siRNA control or siRNA targeting HNRNPH1, were stimulated with IFN-gamma, and were exposed to CAR T cells before imaging.

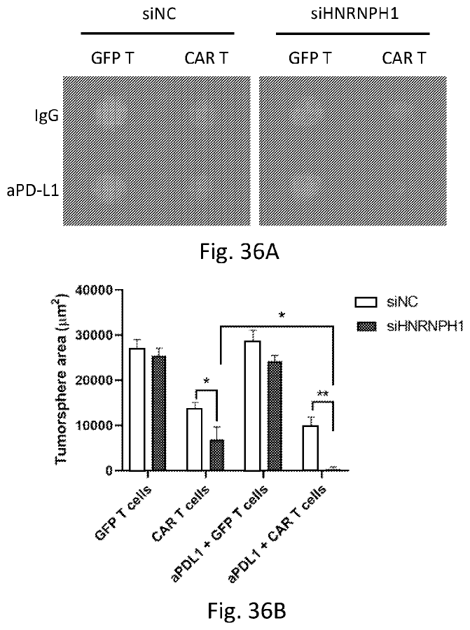

Silencing HNRNPH1 in combination with anti-PD-1 therapy further improve CAR T cell activity (FIGS. 36A-36B).

Taken together, these data indicate that silencing HNRNPH1 improves immune checkpoint blockade therapy.

TABLE 1
Sequences

| SEQ | | |
|---|---|---|
| ID | Descrip- | |
| NO: | tion | Sequence |
| 1 | NEAT1 | GGAGTTAGCGACAGGGAGGGATGCGCGCCT |
| | DNA | GGGTGTAGTTGTGGGGGAGGAAGTGGCTAG |
| | variant | CTCAGGGCTTCAGGGGACAGACAGGGAGAG |
| | MENbeta | ATGACTGAGTTAGATGAGACGAGGGGGCGG |
| | (e.g. | GCTGGGGGTGCGAGAAGGAAGCTTGGCAAG |
| | NCBI Ref | GAGACTAGGTCTAGGGGGACCACAGTGGGG |
| | Seq: | CAGGCTGCATGGAAAATATCCGCAGGGTCC |
| | NR_ | CCCAGGCAGAACAGCCACGCTCCAGGCCAG |
| | 131012.1) | GCTGTCCCTACTGCCTGGTGGAGGGGGAAC |
| | | TTGACCTCTGGGAGGGCGCCGCTCTTGCAT |
| | | AGCTGAGCGAGCCCGGGTGCGCTGGTCTGT |
| | | GTGGAAGGAGGAAGGCAGGGAGAGGTAGAA |
| | | GGGGTGGAGGAGTCAGGAGGAATAGGCCGC |
| | | AGCAGCCCTGGAAATGATCAGGAAGGCAGG |
| | | CAGTGGGTGCAGGGCTGCAGGAGGGCCGGG |
| | | AGGGCTAATCTTCAACTTGTCCATGCCAGC |
| | | AGCCCCTTTTTTTCCAGACCAAGGGCTGTG |
| | | AACCCGCCTGGGGATGAGGCCTGGTCTTGT |
| | | GGAACTGAACTTAGCTCGACGGGGCTGACC |
| | | GCTCTGGCCCAGGGTGGTATGTAATTTTCG |
| | | CTCGGCCTGGGACGGGGCCCAGGCCGGGCC |
| | | CAGCCTGGTGGAGCGTCCAGGTCTGGGTGC |
| | | GAAGCCAGGCCCCTGGGCGGAGGTGAGGGG |
| | | TGGTCTGAGGAGTGATGTGGAGTTAAGGCG |
| | | CCATCCTCACCGGTGACTGGTGCGGCACCT |
| | | AGCATGTTTGACAGGGGGGACTGCGAGGCA |
| | | CGCTGCTCGGGTGTTGGGGACAACATTGAC |
| | | CAACGCTTTATTTTCCAGGTGGCAGTGCTC |
| | | CTTTTGGACTTTTCTCTAGGTTTGGCGCTA |
| | | AACTCTTCTTGTGAGCTCACTCCACCCCTT |
| | | CTTCCTCCCTTTAACTTATCCATTCACTTA |
| | | AAACATTACCTGGTCATCTGGTAAGCCCGG |
| | | GACAGTAAGCCGAGTGGCTGTTGGAGTCGG |
| | | TATTGTTGGTAATGGTGGAGGAAGAGAGGC |
| | | CTTCCCGCTGAGGCTGGGGTGGGGCGGATC |
| | | GGTGTTGCTTGCCTGCAGAGAGGGTGGGGA |
| | | GTGAATGTGCACCCTTGGGTGGGCCTGCAG |
| | | CCATCCAGCTGAAAGTTACAAAAATGCTTC |
| | | ATGGACCGTGGTTTGTTACTATAGTGTTCC |
| | | TCATGGCGAGCAGATGGAACCGGGAGACAT |
| | | GGAGTCCCTGGCCAGTGTGAGTCCTAGCAT |
| | | TGCAGGAGGGGAGACCCTGGAGGAGAGAGC |
| | | CCGCCTCAATTGATGCCTGCAGATTGAATT |
| | | TCCAGAGGCTTAGGAGGAGGAAGTTCTCCA |
| | | ATGTTCTGTTTCCAGGCCTTGCTCAGGAAG |
| | | CCCTGTATTCAGGAGGCTACCATTTAAAGT |
| | | TTGCAGATGAGCTTATGGGGGGCAATCTTA |
| | | AAAAGTCCACAGCAGATGCATCCGGCTCGA |
| | | GGGGCCATCAGCTTTGAATAAATGCTTGTT |
| | | CCAGAGCCCATGAATGCCAGCAGGCACCCC |
| | | TCCTTTCCTGGGGTAAAGGTTTTCAGATGC |
| | | TGCATCTTCTAAATTGAGCCTCCGGTCATA |
| | | CTAGTTTTGTGCTTGGAACCTTGCTTCAAG |
| | | AAGATCCCTAAGCTGTAGAACATTTTAACG |
| | | TTGATGCCACAACGCAGATTGATGCCTTGT |
| | | AGATGGAGCTTGCAGATGGAGCCCCGTGAC |
| | | CTCTCACCTACCCACCTGTTTGCCTGCCTT |
| | | CTTGTGCGTTTCTCGGAGAAGTTCTTAGCC |
| | | TGATGAAATAACTTGGGGCGTTGAAGAGCT |
| | | GTTTAATTTTAAATGCCTTAGACTGGGGAT |
| | | ATATTAGAGGAAGCAGATTGTCAAATTAAG |
| | | GGTGTCATTGTGTTGTGCTAAACGCTGGGA |
| | | GGGTACAAGTTGGTCATTCCTAAATCTGTG |
| | | TGTGAGAAATGGCAGGTCTAGTTTGGGCAT |
| | | TGTGATTGCATTGCAGATTACTAGGAGAAG |
| | | GGAATGGTGGGTACACCGGTAGTGCTCTTT |
| | | TGTTCTTGCTTCGTTTTTTTAAACTTGAAC |
| | | TTTACTTCGTTAGATTTCATAATACTTTCT |
| | | TGGCATTCTAGTAAGAGGACCCTGAGGTGG |
| | | GAGTTGTGGGGGACGGGGAGAAGGGGACAG |
| | | CTTGGCACCGGTCCCGTGGGCGTTGCAGTG |
| | | TGGGGGATGGGGGTATGCAGCTTGGCACTG |
| | | GTACTGGGAGGGATGAGGGTGAAGAAGGGG |
| | | AGAGGGTTGGTTAGAGATACAGTGTGGGTG |
| | | GTGGGGGTGGTAGGAAATGCAGGTTGAAGG |
| | | GAATTCTCTGGGGCTTTGGGGAATTTAGTG |
| | | CGTGGGTGAGCCAAGAAAATACTAATTAAT |
| | | AATAGTAAGTTGTTAGTGTTGGTTAAGTTG |
| | | TTGCTTGGAAGTGAGAAGTTGCTTAGAAAC |
| | | TTTCCAAAGTGCTTAGAACTTTAAGTGCAA |
| | | ACAGACAAACTAACAAACAAAAATTGTTTT |
| | | GCTTTGCTACAAGGTGGGGAAGACTGAAGA |
| | | AGTGTTAACTGAAAACAGGTGACACAGAGT |
| | | CACCAGTTTTCCGAGAACCAAAGGGAGGGG |
| | | TGTGTGATGCCATCTCACAGGCAGGGGAAA |
| | | TGTCTTTACCAGCTTCCTCCTGGTGGCCAA |
| | | GACAGCCTGTTTCAGAGGGTTGTTTTGTTT |
| | | GGGGTGTGGGTGTTATCAAGTGAATTAGTC |
| | | ACTTGAAAGATGGGCGTCAGACTTGCATAC |
| | | GCAGCAGATCAGCATCCTTCGCTGCCCCTT |
| | | AGCAACTTAGGTGGTTGATTTGAAACTGTG |
| | | AAGGTGTGATTTTTTCAGGAGCTGGAAGTC |
| | | TTAGAAAAGCCTTGTAAATGCCTATATTGT |
| | | GGGCTTTTAACGTATTTAAGGGACCACTTA |
| | | AGACGAGATTAGATGGGCTCTTCTGGATTT |
| | | GTTCCTCATTTGTCACAGGTGTCTTGTGAT |
| | | TGAAAATCATGAGCGAAGTGAAATTGCATT |
| | | GAATTTCAAGGGAATTTAGTATGTAAATCG |
| | | TGCCTTAGAAACACATCTGTTGTCTTTTCT |
| | | GTGTTTGGTCGATATTAATAATGGCAAAAT |
| | | TTTTGCCTATCTAGTATCTTCAAATTGTAG |
| | | TCTTTGTAACAACCAAATAACCTTTTGTGG |
| | | TCACTGTAAAATTAATATTTGGTAGACAGA |
| | | ATCCATGTACCTTTGCTAAGGTTAGAATGA |
| | | ATAATTTATTGTATTTTTAATTTGAATGTT |
| | | TGTGCTTTTTAAATGAGCCAAGACTAGAGG |
| | | GGAAACTATCACCTAAAATCAGTTTGGAAA |
| | | ACAAGACCTAAAAAGGGAAGGGGATGGGGA |
| | | TTGTGGGGAGAGAGTGGGCGAGGTGCCTTT |
| | | ACTACATGTGTGATCTGAAAACCCTGCTTG |
| | | GTTCTGAGCTGCGTCTATTGAATTGGTAAA |
| | | GTAATACCAATGGCTTTTTATCATTTCCTT |
| | | CTTCCCTTTAAGTTTCACTTGAAATTTTAA |
| | | AAATCATGGTTATTTTTATCGTTGGGATCT |
| | | TTCTGTCTTCTGGGTTCCATTTTTTAAATG |
| | | TTTAAAAATATGTTGACATGGTAGTTCAGT |
| | | TCTTAACCAATGACTTGGGGATGATGCAAA |
| | | CAATTACTGTCGTTGGGATTTAGAGTGTAT |
| | | TAGTCACGCATGTATGGGGAAGTAGTCTCG |
| | | GGTATGCTGTTGTGAAATTGAAACTGTAAA |
| | | AGTAGATGGTTGAAAGTACTGGTATGTTGC |
| | | TCTGTATGGTAAGAACTAATTCTGTTACGT |
| | | CATGTACATAATTACTAATCACTTTTCTTC |
| | | CCCTTTACAGCACAAATAAAGTTTGAGTTC |
| | | TAAACTCATTAGAATTGTTGTATTGCTATG |
| | | TTACATTTCTCGACCCCTATCACATTGCCT |
| | | TCATAACGACTTTGGATGTATCTTCATATT |
| | | GTAGATTTAGGTCTAGATTTGCTAGCTCCA |
| | | AGTAATTAAGGCCATGTAGGAGAGCATGGT |
| | | AACCACAGATAGAACTGGTATTATCCCAAG |
| | | TGGTCTGCAGACTGCTGAGTGGGGATGGGA |
| | | TCTGCTCTCTGTTGAGAGTTGGTAATCATT |
| | | GGTTTGAAATGTGATGAAACCACTCAAGCC |
| | | AATGAAGGTGGGTGTGTAGGTGGGGAGTAC |
| | | TTTGCCATAATATTTTAAAACATTACCTGG |
| | | TTAGAGTTCTAAGTGGTACTTATTTTTGTT |
| | | TGGTTAGGGGAAAGCCTGAATAAAAACAGA |
| | | AATGGACACATAATATGCATATTCCATAGT |
| | | CTTTGGGAGGCTGGAATGTGCCTGGGATTT |
| | | GGGTCTAAGTGTATGCGTAATTCTTACCTC |
| | | ACTAAAGAATTTGCCTTGTTTTTTTCCTTT |
| | | TGGTGAGTGACTAAAACGTCTGGGCTTCCC |
| | | TGTGTGCGTGCTACAGTAAGCAAGCAGAGG |
| | | CTGTGCAAAGGTGTGAGCAGGATCACGTGG |
| | | AATCTGGAGGATACATCTTGGCTTGCAAAC |
| | | TGCCTCTGTCTCCTGGGTGGGACTGTTCTG |
| | | TCCTTGCACTGCTGTTCTGTGTTACCTCTT |
| | | GGGGTGTAAGGTTTTGCTTACAGGAGACAA |
| | | ACTTTGGGCGTAGAATGGAAGCCACTGCCA |
| | | GCCTCTGTGCTGAGAAGGAAGGTGCTTGTT |
| | | TCAAAGGGAGCAGCAAGGGAGGCTTGTTCT |
| | | ACTCACCTGGGCCTGTTTGCCTGAGAAGGG |
| | | GAGATAAGGGCTGAACTGGGACTAGCCAGG |
| | | GGGACCAACACAAATGGTGGGGGATCATGA |
| | | CCTGAAGGATTCTTTCCTTCCCATGAGCTG |
| | | CAGGGCTGGTTGCCGTCCTTGCAACTGTGT |
| | | CTTATTTGCCTGTGCCGTTATATCTTGGTG |
| | | ACCCCTCCACGTGTACACTACTGACAAACG |
| | | GGTGGAGTGCTGGGGAGAAGTCACTGTGCC |
| | | GCCCACCTAGTAAACCTTCTGTCTGTGCTC |
| | | ATGGCATCTCCAAGATGGGGCACTGCTGTG |
| | | TGCAGAATCCAGGGTCCTCTTTCTGCTTGC |
| | | AACTCCTTTCCCTGGATGCCCCAGAAACAA |
| | | TCCAGGCCTCCTTTCCTATCTTACCCCTTT |
| | | GCTTTGCTTTTTACCCCAGCACCTCTATAA |
| | | CCGCCTTCTCTTCTTTTCAGAACTCCTTGT |
| | | TTCTCGTCCTGTTTTTTATGATTACAAAAC |
| | | TCTTGCTTCCACCCTGGAAGATAACTGCTA |
| | | TAGATGCCTGTATGTAAATGGTGCTGTCTC |
| | | CAGCAACTGGCATGCTGAAGAAGAATTGAT |
| | | TCACGGGGTATAAATGTTGGGGATTGGAAG |
| | | TGGGGATGAAATGGCACTTGTTGATACAGG |
| | | AGCAGAGAGGTGAGGCCGACTGCTGAAGAC |
| | | AGCTCGCCACCCTCCTTGCCTCCACTCCAA |
| | | TCCAGGGGCTGGGGCCACATTCTTTGCCTT |
| | | CATTTATCCTCAGATCAGGTGAGATCGACA |
| | | GGAGGTGTTGATGGCAGTGCCAGCAATTAT |
| | | TGCTAATCCGTTTGCATCCTTATGCATAGA |
| | | TCTGAATTCAGACTTTGTGAATTTCCAGAG |
| | | GTGTGGGTAATATAATAGAATTCAGTGAGT |
| | | GGGCATGGCTGATCTTGTGCAAATTAAAAG |
| | | TTATGGGGCATAAGAATAGCAAAAGTTGAA |
| | | CTTCTTTTAAAAAGGAAAGTACCCTGAGAG |
| | | CCAGTATTGGTTGAGGCTCTTCAGTATGCC |
| | | CAGGTTGGCAGCACTGAGAACCGCAGGAAC |
| | | GGCCTGTTGTTACAAAAAGGAGATTGACTC |
| | | AGCTGCCCTTGGTGCATCTGACTGACTATG |
| | | ACTGCTGAGAGATTCCAAGGACCCTTAATG |
| | | CCAGGGCTAACCTCTCCATGTGCAGTGAGA |
| | | CCTCTGGAGGAAGTGTCATCCTCTGGCTTT |
| | | GTGTGGTACTCATTATGGTGCAGTGCGGGC |
| | | ATGAAATGAAGACACCCAAATAGGCTTACA |
| | | GATACGATATGTTTTAAATGTTCGTATTTA |
| | | ACAAAAACATACTGACACTGTTTGGAAATG |
| | | GCAACAGGAAGATAGCAAAATGAATACTAA |
| | | CATTACGAAAAGATGAACAGGTACATGTTC |
| | | CAAGGCAGGTGGCTGTGAACTTCCTCTGAG |
| | | TGAAGGCATCCCCTCCAGCACCTTTCAGCC |
| | | TGCTAGTTAGGACGACCCGCCGCCACCCTC |
| | | CAGGACCTCCAGCCCTGCACTGCCTTTCCT |
| | | CTCTTTTAAATAATTCTTCATTGAGTTCTA |
| | | ATATGTAAAAAAAAAAAGTTTACTGTAAAG |
| | | TTTGCAAATAAGGAAATTTTTTTTAAAAGT |
| | | CCTCAGTAATCTTACCAGTAACAATTGTTA |
| | | TGGGCACATTTGCTTTTGGAAGATTTCTTT |
| | | TGTATGCATGGGATAAGTACATTTTTAAAC |
| | | AAAAATGGGATTATGCCATAAATTCTATTT |
| | | TGTGACTTTAATATATAGTGAACACCTTTT |
| | | TTAATGATGACAGGATGTTCCCTTGCATGG |
| | | CTGTATCAATTTAAACAATCTTGTTTCAAT |
| | | GGGCATACAGGGTATTTTCTAGTTTTTTTT |
| | | TCCTCTTAGAAAATAATACTTGCGATGACT |
| | | TTCCTTGTAGCTCAGACTTTTTCACGTCTG |
| | | TTGTTATCTCTTTGGGAATGCTGAATACAT |
| | | ACATTTCGAGAAGGAAATGACTGTTAAACT |
| | | CTTAAGACTTCAGGTTCATATTGCTAAACT |
| | | GCCCAGCAGGGAGGGATTTTTTCAATTAGT |
| | | GTTCTCACTGGTGAGGCAAACCTGATGCCT |
| | | TCCCCTCTTCCTCAGAACCGGCTTTATCAC |
| | | ATTGAAAACCTTTGCTCCTCCGACGGATCG |
| | | AGTCTGCTTTCCCTCTGGATGTGAGCATTG |
| | | CTTTGTCTGCTGGTGACTGAACATCTCTAC |
| | | CTTGTGTCAATTGGCCATTTGTGGTGTGTG |
| | | TGTGTGTGCGTGTGTGTGTGTGTGTGTGTG |
| | | TATGATTTTCTAATTCCTAGTCATTTTTCT |
| | | ATTGATTGTTTTGCAAAAGCCATTTACATC |
| | | TTAAGGATATTGATAATCTTTTGTTATATT |
| | | TGATGCAAATATTTTTTTCCAGTTTATAGG |
| | | TTGCCTTTTAATTTTGTGTTTCAGGTAGAT |
| | | AAAAGTTAAACGATTTTCTTAGGTTAGTTT |
| | | ATCACTGTGGTTTCTGAACTTGTTATGTGT |
| | | AGATCTTTTCCACCCCAAGAGTACATAAAT |
| | | ATTAATCCATACTTTCTTATGGAACTTGTA |
| | | TGGTTTCGTTTTTTACATTTAAACCTTCTT |
| | | CCCCGTGGTGTGTGTTGTGGAATCTGTGTT |
| | | TGTGTGAGGAGGGGCATGGTGCTCTCAGAA |
| | | CCCACCTCCTGTGGCCAGAGAGCCCTGTCC |
| | | TGTGAGGGTGGTTGTCACAGTGGCAGGGTT |
| | | CAATTCAGAAGACCTTGAGGGCAGGCTGAT |
| | | GTTTCCTGAATGGGCCCCTGGTTGTTGCTT |
| | | GTCCCTGACTCTCCATTTCCCCATCTGAGT |
| | | GGATTTGGACCTAATAGGGCACTGGAGCTG |
| | | GTTCGAATCCTGACTGGACTACTTGGCAAC |
| | | TTTATGTCTGGGAGCAAGTTACTTAACCTC |
| | | CCCAAGCCTGTGTCTGTGAAATGCGGGTAA |
| | | ATGAATGTAGATGTTTGGCAGCAGCTACTC |
| | | CTTGTTGAGCTCTCACAGTGAACTCTCCTG |
| | | CCTCTGCCCTCCTTCCCCGCCTCCCCTGGT |
| | | GCCTAGCGTCAGGTCTAGCCACTTCCTCCT |
| | | GGGCCCCTCTCCCTTTTCTGTGGCTGGCTG |
| | | CCTGCCCGCCTGGCGCTGGACCTTTCATGT |
| | | AACGGGAATCAGCATGTATATTCTGGTCTG |
| | | GTCTGTTTCTACACTTAATTTTGTTTCCAG |
| | | TAGTATTTCCCTGTACCGGCAGAGTTCACA |
| | | AACACATTTGAAGAGGCTTTTTCTCAGGAT |
| | | TCTTAACCTTCCCAAAGGAAGTCCCATGGA |
| | | TGGGTTTCTAGAAGTCTATAAATGCTCTGA |
| | | AATTGTATTTTTCTGTGGAAAGCATAACTT |
| | | TCATCTGCTTGTTCGTGCTCAAAAAAGATC |
| | | ATGAATGAATGATTGCATGATTTTATGCCA |
| | | TTGTGCTTATACTAAAGGATATGTAGCCCA |
| | | TCTCTTGAGCTGTTAAACTGTTTTGACTAC |
| | | TTTAAATCGTGCAGCTGTGAGCATCTCTGT |
| | | AAATTTAGTGTACACATGTATCCCCTGGAG |
| | | TGGCATTGCCTCGGCAGTGAGCACTTATGG |
| | | TTTTATAACTCTCTTCACAGACTCAAATGA |
| | | CTCCAGAAAGCTACACTTCCTGTTGTGAGT |
| | | ATATGATATCCATTTCCCTACATAGCCACT |
| | | AACATCAGGTTTTTACAATTTTATTTATTT |
| | | CTTGCTACTTTAAGAAATTTTTGTGGTGAA |
| | | ATACATATAATAGAAGTTGACTATCTGAAT |
| | | CATTTTTAAGTATACATTCAGTAGTGTTAA |
| | | GTATGTCGCCATTGTTGTACAACCAATCTC |
| | | CAGAACTTTTTCATCTTGCAAAACAAACTC |
| | | TGTACCCATTAAATAACATTAAACATTCCA |
| | | TTCCCTCCAGCCTCAGCAACCCCATTCTAC |
| | | TTTCTGTTTCTGTGAGTTTGACTATTCCAA |
| | | GCACTTCATATCAGTTAAATCATGAAGTAT |
| | | TTGTCTGTCTGTGACTGGCTTATTTCTCTG |
| | | AGCACAGTGTCCTCGAGATGCGTCTATGTT |
| | | GTAGCATATGTCAGAATTTCCTTCCTTTTT |
| | | AAAAGATCCAAATAATATTCTTATTTTATA |
| | | TCTTTTTTTTATCCATTCATCCATTAGTGG |
| | | ACACTTGGGTTGCTTTTGGCTATTGTAAAT |
| | | AATGGTGCTATGTACAAATATCTATATTAT |
| | | TGTATTTACAAGTATAATGCTGTAATGTAC |
| | | ACACATCTTTTTGAGATCCTACCTTCAGTT |
| | | CTTTTGAGTATATAGCCAGAAGTGGTATTA |
| | | CTAAATCTTACGATATTTCTATTTTTAATT |
| | | TATTGAGGAACCACTGTAGTTTTTCATAGC |
| | | AACTGCACCATTTTACGTTCTCACCAAGAG |
| | | TGCACAAGGGTTCCGAGGTTCCCACATCCT |
| | | CCCCAACACTTGTTATTTTCTGCTTTTTTT |
| | | AGATTGCAGCCATCATAGTGGGTGTGAGGT |
| | | GACATTTCATTGTGGTTTTGATTTGCATTT |
| | | CCCTAATGAGGAGTGATGCTGAGCATCTTT |
| | | TCATATGCTTACTGGTCATTTGTATGTTGT |
| | | CTTTGGAAAAATGTCTATTCAAGTCCTTTG |
| | | ACTATTTTAAAAATTGGGTTATTAGAGTTA |
| | | TCGTTGTTGTTGACTTGTAGGAGTTTCTTT |
| | | CTATATTCTGGATATTAATCCCCTATCAGA |
| | | TATATGATTTGCAAATATCTTCTCTTATTC |
| | | CATAAGGTTACTTTTTCACTTTGTTGATTG |
| | | TGTTCTTTGATGTATAGAAGTTTTTAGTTT |
| | | TGAAATAGTCTAATTTATCTGTTTTTACTT |
| | | TTGTGGTCTGTGCTTTTGGTGTCATATCCA |
| | | AGAAATCCTTGCCAAATCCAACGTTATAAG |
| | | GTACTTTTAAGGTATTTTAGTTGTCTTAGT |
| | | CTATATTTCTGTACTCACCTTTCTTTATCC |
| | | ACTCATCAGTTGATGGGCATGTAGGTTGGT |
| | | TCCATATCTTTGCAATTCTGAATTGTGCTA |
| | | TGATCAGGTGTCTTTTTAGTATAATGATTT |
| | | ACTCTCCTTTGGGTAGATACCCAGTAGTGG |
| | | GATTGCTGGATCGAATGGTTTTTATAATTT |
| | | TCTATTTTACCACAGTTTCTCTCTGCATTT |
| | | TTCCTCTTTGACCACTAACCATGTGAAATT |
| | | CTCATATTGACCTTTATAATGATCATGAAC |
| | | TCTTAGTATCATTGGGAAGGCCACATTTGC |
| | | CACTTATGATTGTAAACCTTATCCTCCATT |
| | | TTTCCTGTTATTGTTGGTGCAAAAAGCACC |
| | | TATTATACCAGGACTTTAAAAATCAGTCTG |
| | | ATAAGTCTTTGATAAGTCTAATAATAATAA |
| | | CTGATAAGTCCATTGAATTTGCTTCTGATT |
| | | ACTTTTTCTTTAGTAGCTAAACATGTATGT |
| | | ACTCCTATGATTACAATGAACACTCCTCTC |
| | | CATTTAAATTAATTATTTACATTGATGAAA |
| | | TAGCAAAATGTTAATGACTAAATACTGTCT |
| | | TGGTTTTTTCGTTCCAGGTCAGTCAATATT |
| | | AACTTCTTATAATTTTCTTTTTTTTCTTTA |
| | | TGTGTGTGTGTGTGTGTATTTTTTTTTTTT |
| | | TAATTTCAATGGCTTTTGGGGTACAAATGG |
| | | CTTTTGGTCATATAGATGAATTCTACAGTA |
| | | GTGAAGTCTGAGATTTTACTGCACCGGTCA |
| | | CCTGAGTAGTGTACATTGTACCCAATATGT |
| | | GGTTTTTTATACCTTGCCCCCCTCTTACCC |
| | | TCCCCACTTTGAGTCTCTAGTGTCCATTAT |
| | | GTCACTCTGTATACCTTTTTGTACCCATAA |
| | | GTTAGCTCTCACTTATAAGTGAGAACACAC |
| | | AGTATTTGGTTTTCCATTCCTGAGTTGCTT |
| | | CACTTAGAATAATATCCTCCAGCTCCATCC |
| | | AAAATTGCTGCAAAAAAAAAAAAAACCACA |
| | | AACATTATTTTGTTCTTTTTTATTGCTAAG |
| | | TCATATTCCATGGTGTAGAGATACCACATT |
| | | TTATTTATCCACTCACTGGTTGATGGGTTG |
| | | GTTCCACATCTTTGCAATTGTGACTTGTAC |
| | | TGCCATCAAGTGTCTTTCTGGTATAATGAC |
| | | TTCTTTTCCTTTGGGTAGATACCCAGGAGT |
| | | GGGATTGCTAGATCAAATGGTTCTTAACAT |
| | | TTTCTCTCTGGATCTATTTCTGGAAATTTT |
| | | AGGCTCCAGTTTTTGTTGTTGTTGTTAATA |
| | | AAATGCAATGGAATGTAATGATCATCACTT |
| | | TTCATTATGCTTTAAAATCTGGTAAATGGA |
| | | GGCTAGAACACTCCTGTAAGGCAAGAATAT |
| | | TCTCTCTGTTGGAACTCAAATACACAGAAC |
| | | TGGGTAAATCTCAATCTTAATCTTTGATTC |
| | | AGGACACAACATGGCTCTCTTTTACTTGCT |
| | | TTCTTTAATTGTTTTTTAATAATGTGGTAA |
| | | GCATTTCTGAATCTCCTATCCAATACAAAA |
| | | ACTAGGACAATACAGACAGTAACTCCTATG |
| | | GTTACAATGAACACTCCTCTCCACTTAAAT |
| | | TAATTATTTACACTGATGAAATTGAAATAG |
| | | CAAAATTTTAATGACTAAATACTGTCTTTG |
| | | ATTTTTTGTTCCAGGTCTGTCAATATTAAC |
| | | TTCTTATAATTTTCTTTTTTTTTCTTTATG |
| | | TGTGTGTGTGTGTGTGTATATATATATATT |
| | | TAATTTCAATGGCTTTTGGGGTACAAATGG |
| | | CTTTTGGTCATATATATGAGTTCTACAGTA |
| | | GTGAAGTCTGAGATTTTACTACACCTTCCA |
| | | CTTATGTGGTCCCACACCACCCGCCTCCCC |
| | | TGCCGCCTCCTGCCACCCCCTAGGCCAAGG |
| | | TAATAATCATCCTGAATCCTGGGTTTATCT |
| | | CTCACTTGCTTTCTTTTCATATAATTTTGC |
| | | AAAAGAATCTGATCTAAATGTGTTTTTCAG |
| | | AGTATATATTTATATTTTAGCTGTTCTTAG |
| | | AGAAAATTTATTATTTTGCATGTAATCTTA |
| | | TGGAACATTCTCATTTAATACCATGGTAAG |
| | | ATTCAGCCCTTGCCCAGGGGATAGTTCATT |
| | | TAGTTTGTTTACTGGATAGAGCTCATCATG |
| | | TGACTATACCTCAGTTAGTTTATCAGTTCT |
| | | CCCATCCATGGTGACTAGGTTGCCTCTCAG |
| | | CCTCTCAACAACACTGTTTCTCAGTGTCCT |
| | | TGTAGAAGTGATATGTGGGTGTTTTCTCCT |
| | | TACACAGAGTTGAAAGGTGACGACAACAAC |
| | | GTTGGCACTACCAATCCCCCACCCTCCAGA |
| | | GGGGTAACCAGTGTTACCAGTTTGCTGTGT |
| | | TTCCTGCTACACCTCGCCTTATTCACTTCC |
| | | ATTTGTATCTGAAAAACGTGTTGCATGGTT |
| | | TCTTTTCTATAGAAGTGGTAAAATGCTATT |
| | | GTGTCCTGTACATTATTGATTACTTTTTTT |
| | | CATTTAACAGTAGGGAGATGCCTGGGAGTA |
| | | CACAGAGAACTGCCCTCATTGTTTTCAACT |
| | | TCTGCACTGTATGTCTGTGAGTTTAGCCAT |
| | | TCTGCTGTTAATGGAAATTTACAGTATTCT |
| | | AATCTTTTGATATTACAAACAGTTCTGTGC |
| | | GATCATCGTCATACACAACCCCTTGTGCAC |
| | | AATGCATGAGTGTTTCTCAGGGTAGGTACC |
| | | AAGAAGTGAAATTCCTGGGTCATAGGGCGT |
| | | GAGTCCGACATTTTTCTCCATTCTGCCCTG |
| | | TTGCCCTCCAGAGTGGGTGTCCAGCTTTGC |
| | | ATACCTAAGTATGAGAGTATCTGTTGTTCA |
| | | TATCCTCTACGACGCTCCATATATGAAACT |
| | | TAAGTTTCTGCTAGTTGCCATCTTTGATCT |
| | | ATCATGTATGCAGTGACCTACTAAGACTGT |
| | | AATTGGTACAGTAGATTCTTGTCATCTGTG |
| | | TGTGAATTTAGCATTCATGGGCTTAATGCT |
| | | GACAAGGCCCCCAGGGTCCAAGACATATAA |
| | | TCATGTATAATTTTGTCAAGGTATAATTTT |
| | | TTAAATTGCTTTTGTCATGTGTCTGCTGGT |
| | | GATGCCCAACCCAGTGCTCTGCACCCAGGT |
| | | CACACTGTGGCTTTGTCCTCTGCTTATGCC |
| | | TGCATTGCAGCAACTGTCCTGAAGAGACCA |
| | | AAATTATGCAGATTTAGGTAAGTCCATGGC |
| | | TAATGTTATTATATTATGTGCTATTGTAAT |
| | | GGATGGGGCTGTGGAGTGTATGAATTTATA |
| | | AATCACTGGTCTTGTAATTAAAATTCAAAC |
| | | ACTATAGAAAAAGGCCATGTAGAAGATAAA |
| | | AGTTCCTCTATAATCCCGGACCCCTAAGAT |
| | | AACTACTAATGACAACTTCATTTATATTCC |
| | | TTCAGACATTTTCTGGCTGTGGATGTACTA |
| | | AAATGTATCCTATTATTCTCTGCCCTAAAA |
| | | TGGAATCATACAAGGTGTACTGTTATTTTT |
| | | ATGGCTCTATAACATGTCATATTGTACGTG |
| | | TTGGTATGGTCATTTTAACCATTTTTCTAG |
| | | TGATGGCTTTGAGGTTATTTGCAGTTTCCT |
| | | AGCCATCTCAAAGTGTGCTGCGGGGATCTC |
| | | TTTTGCATCCCTCTGGGTGCAGAGCTGAGG |
| | | CACCCAGAGGCAGTGTCCAGAGGAGGCAGC |
| | | ATCTGTAGGTGTCTTCACCTGCTCTGGCTC |
| | | TTGGCACATCTGGTTGGTGACACTGTTTTG |
| | | TGAGATGGGTTGAAAGCACGTGCTGCCAAA |
| | | ATAGAATAATGTTGGTCCTCTCCTCATGTG |
| | | CCGTGGAACTGGGGTAAAACTGCGTAGTGG |
| | | CTGCAGCTGCCTGTCCATACCGGAATCGAG |
| | | TATAACACGGTGCCTGGCTTAGCACAAAAC |
| | | AGTAGTGGGTCCTGCAGGCCCCAGAGTCTA |
| | | ATTCCTGGTATTCTTTCCCCTACACAGATT |
| | | AAATAAACCAAAAACAAACTATTCTAGGAA |
| | | AGCGTCTGTGACATTTGTAAAAAGTGGTAT |
| | | TTAATGATCTTTTATTCACTTGTCTGTTTA |
| | | GTTTGTTGAAATCTTAAGTGGCATCCTGGT |
| | | CTGGGAAGGAGTGCTGTCTGCGCCTGCCCT |
| | | CCGCTGGGCACAGCGTGGCTGCTTCAGGGG |
| | | CTAAGCACACACTTTCTGTCTTCTAAAGGG |
| | | CCGCCACATGCCAGGAGCTCAGGTGTGAGC |
| | | CCGGCTCTGGCTCTTACCTCATAGGGTCAC |
| | | TCATAGGGGCACAGGGAGCAGAACATTGTA |
| | | CACAGCGAGGCACCACCCGGCTTGGCATCT |
| | | GCCTCGGTGGACTTACTACCTCTAGAAGGA |
| | | AATACCTGAGTTCCTCTGGCCTCAGCTCCT |
| | | AGAGTGACTGGTGTGCTGTCCCTGTTACTC |
| | | TTCTGTCAAGGTGACAACTGTGTGACCCAT |
| | | CATCTGTGTGTCAAAGCAAGGCCCTGCCTG |
| | | GGCCTCTGCTCCTGTGCTGACCCCAAAGGC |
| | | AAATGCTTTGCTAGTTTCCTTCCAGTTAAT |
| | | TTCACCTATGAATAGATGTGTGAAAACTGT |
| | | TCAAAGCCATACCTGCACATGTTTGAACTT |
| | | CAAACCCTGTGGGTGATTCAGTGGCATCTT |
| | | TCTCTAACCCCCAGCCTCCCTTCCCACAGA |
| | | GGCCACCGTCATGGCCAGTTGCTGCAGTTT |
| | | CTTTCCAGAGAACCTGTGTATGTGTAAAGC |
| | | TGTACAGGCGTGGGTACACCACACAGCCTG |
| | | TCTTGCACTGTGGACTGTTGAGTTACTAGT |
| | | ACATCTAGGTAAGCACCGCATATCTGTATT |
| | | CATGTCTGCCTTGGTCTTTTCAACATCTGT |
| | | GTGGTAGCCGTGTTTGAATTACCCATTCCC |
| | | TTTTTGGGGAACCATTAAGTTGTTTCAGCA |
| | | ATTTTTACTGTAGATAAGGCTATACCGCAT |
| | | ATCTGTGTACATGGGTTTTTATGTACATGG |
| | | GCAAGTATATCTGTGAGAGAAAAGTTTCCT |
| | | CAGGAGGAATTCTGGGCACAGCATGTGTAA |
| | | ATTTCTAAATATGATGGACACCCCCAGCTT |
| | | CCACCTCAAGGAGGTTGGTCCCATTGACAT |
| | | TTCCCCACACCTTCACCCAGGCTGTGCCCT |
| | | TAAACTTGGTTATTTGTCAATGTGAGAAGT |
| | | GGAAAATAGTATTTAATTGTAGTTTGGATT |
| | | TGTATTTCTATTGGGTTGTATACTTACTGA |
| | | TTAATAATAAGAGCTCTTTACATATTAAGG |
| | | AAATTAACCCTTTTCAAATACATTCCTATT |
| | | TCTCACTAATCTTTAAGTTTTATTGTAATA |
| | | TTTTGCTCTTTAGTTTATATATATATGTAT |
| | | ATATATATATATGTATATATATATATATAC |
| | | ATATATATATACATATATATATACTAATTT |
| | | TCTTTTATGGTTCCTGGATTTTGTGAGTAG |
| | | TTTGAAAAGGCTAATCCAGCTGAAGATTTT |
| | | GTTGTTGTTGTTAAACCCCATGTTTTCTCC |
| | | TAACTCTTTTTATTTTTATTTTGGAGGACT |
| | | CTATCTAGACTTAATTTTAGCATAACAAGT |
| | | GACAGGGTTAGTTAGCCTGTTGTCCTTACA |
| | | CCATTTTCTGGCTAATACAGCTATTAACTA |
| | | TTGATCTGTCTATTCACGTGCCAGTTCCTA |
| | | ATGGTTTTACATAGTGTAATCTGCACTTCA |
| | | AAATAGCGAAGGGAAGCCCTACCTCATTAT |
| | | TCTACTTTTCCAGAATTCTCCTGGCTATTC |
| | | CAGGCTGCATGTTTACCTTAACCTTCCCTG |
| | | TGATGTCTTCATGCCGTTGTCTTCTTATGC |
| | | AAGAATAAGGTACGTCTTTCCATCCACTCA |
| | | CGTCTATTTAATTTGACTTTGCATTACACA |
| | | GAAAGCTGGTCTTGGTCTGTCTACCTCGGC |
| | | ATCTAGTTGTCCTCACTGCCCCCTAGCCGA |
| | | CCCCACCCCATCTGACTGACTACCCCATCA |
| | | CAGAGTACTTTTATTTACGTTTTGCTCTGC |
| | | CTAATGGTTACTTGATACTGTCACGCCGAC |
| | | AGTGTCCAGTTCAGTGGTCTTTGCAGTTGA |
| | | AATGCTCCCGTACACACTGTCTTGTTAAAA |
| | | ATGCCAGTAAGTTCATACAAACCCAGCTTG |
| | | CACCCAAGGTCACATTCAGAGAGCGTAGGG |
| | | CTGGGATGGGTTGTTTTCCAAGCTTCTGCC |
| | | ACTGTGTGGCTAGCTCTTCCCACTGGGAAG |
| | | TTCTGTGTACCCGGAATGTCGGAGTGGAGT |
| | | CCTGTTCTAGTGTCCAGCACCTGACCCTGT |
| | | GCCCAACCCCTCAACAGCCTATTCCTGCTG |
| | | TCCACAGCCTGCTGGAACTTTTTACAAAAT |
| | | ATGTTGCCATGCTGGACCCTGGGCACTGGA |
| | | CATAAGCCCCCTGGCAGCCTTTTTCATGTC |
| | | ACCCAAAGGGGTAATTGTCCTACTGGTGGT |
| | | CTGTAAGATGAGTTAGGGTGACTTGCTAAT |
| | | AGACATTGTAAATCTTAATATTTATGTATG |
| | | TATTTTATTATTACCGGTTTTCCATTTATG |
| | | ATGGTAATATTGTTTCTTCTAAGAATATTT |
| | | ATTTTTCCTTCTAAATATTGAGATAAAATT |
| | | CATGCTTTTGAAATGTTCTATTCAGTGGCT |
| | | TTTAGTATATTTGCTATGTTGTGCAACCAT |
| | | CGACACTATCCATTTCTAGAACTTTTTCGT |
| | | CATCCCAAACAGACGCTCTGTATTCATAAA |
| | | AAAATAACTTCCTACCTGTCTCTCCCCCTA |
| | | GTCTTTGGTAACCTTTGTTATACTGGTAAA |
| | | CTTTGTTGTGCTCTCTGTCTGTGTGAATTT |
| | | GCCTATTCTAGGGGCCTCATATAAGTGTAA |
| | | TCATACAGTATTTGTCTTTTTGGGTCTGTC |
| | | TGATTTCACTTAGCGGGTTTTCAGGGTTCA |
| | | TTCATGTTGCAGCATATAACAGTACTGCGT |
| | | TCCTTTTTCTGGCTGAATAATATTCCACTG |
| | | TATGGATAGACCCCATTTTGTTTATTCACA |
| | | CATCATTTGGACATTTGGATTATTTCTGGT |
| | | TTTTGGCTATTATGAACAATGGTGCTATGA |
| | | ACAGTTGCGTACAAGTTTTTGTGTGAACAT |
| | | ATGTTTTCAATTCTCTCATTATATACCTAG |
| | | GAGTAGAATTACTGGGTCATATGGTAACTG |
| | | TATATTTTTGAGGAACTGCCAAACTATTTT |
| | | CCCACGTCCATGCACCATTTCACATTCCCA |
| | | CCAGTAAGTAAGAGGGTTCCAATTTCTGCG |
| | | CATTCTTGCCAACACTAGTTATTATCTGAC |
| | | TTTCTGGTTATAATCATTCTAATGAGTGTG |
| | | AAGTAGCCTCTGGTGTCATTTGGATTTGCA |
| | | TTTCTCTGATGAGTGATGCTATCAAGCACC |
| | | TTTGCTGGTGCTGTTGGCCATATGTGTATG |
| | | TTCCCTGGAGAAGTGTCTGTGCTGAGCCTT |
| | | GGCCCACTTTTTAATTAGGCGTTTGTCTTT |
| | | TTATTACTGAGTTGTAAGAGTTCTTTATAT |
| | | ATTCTGGATTCTAGACCCTTATCAGATACA |
| | | TGGTTTGCAAATATTTTCTCCCATTCTGTG |
| | | GGTTGTGTTTTCACTTTATCGATAATGTCC |
| | | TTAGACATATAATAAATTTGTATTTTAAAA |
| | | GTGACTTGATTTGGCTGTGCAAGGTGGCTC |
| | | ACGCTTGTAATCCCAGCACTTTGGGAGACT |
| | | GAGGTGGGTGGATCATATGAGGAGGCTAGG |
| | | AGTTCGAGGTCAGCCTGGCCAGCATAGCGA |
| | | AAACTTGTCTCTACTAAAAATACAAAAATT |
| | | AGTCAGGCATGGTGGTGCACGTCTGTAATA |
| | | CCAGCTTCTCAGGAGGCTGAGGCACGAGGA |
| | | TCACTTGAACCCAGGAGGAGGAGGTTGCAG |
| | | TGAGCTGAGATCATGCCAGGGCAACAGAAT |
| | | GAGACTTTGTTTAAAAAAAAAAAAAAGTGA |
| | | CTTGATTTAAGGGAAAAAATGACTGGCTAT |
| | | ATTCAGTCAGATATGGCAAAAAGTCTCAAG |
| | | GTGTTAATGTGAATGATTAAGGTCTTGGGG |
| | | GGGGTGTCCCCTATCAGACTACAGGTGTTT |
| | | AGAGGCACAGAAAAAGGTGCAGTTGGGTTC |
| | | TTAATGTGAAATGATGAGAAGCACAACTCC |
| | | AGTGTGTCTCTTTGTGTAGAATGTCAGCAG |
| | | ACACCCCCTGCTAGATGTGCTGGATCATGG |
| | | GAAAGCATTTCCATTTGTTACTAGATTGTT |
| | | CAGAAGTTTTAATTTATGATGGGTGTGGTG |
| | | GCTCATGCCTGTAGTCCCAGCACTGTGGGA |
| | | GGCTGAGGCAGGAGGATCATCTGAGGCCAA |
| | | GAGTTCAAGATCAGCCTGGGCAACATAGTG |
| | | ATACCCTATCTCTTAAAAAAGAAGAAGTTT |
| | | TTAAATTTGAAATAATAATAGGTACTGGAT |
| | | TTATGCAAATGTCTTTTCTGCGTCTTTTGA |
| | | GATGAGTATCAGGTTTTTTTTTTTCCTTTT |
| | | ATCATCTGATGATGAACTTAATGTTTCCAT |
| | | TTGTATTAATGGAATACTAAGTCCCTCTGT |
| | | GATTTCTGAACCAAGCTATTCCTAGGCCTG |
| | | AGTTTTATTTTGTTGACACAGAAATAAATT |
| | | AGAAGGCCAAGCGTGGTGGCATGTGCCTGT |
| | | AGTCCTAGTTGCTGAGGTAAGAGGATTGCT |
| | | TGAGCCCAGGAGTTCAAGGCTGCAGCAAGC |
| | | TTTGATTGCGCCACTGCACTCCAGCCTTGG |
| | | CGACAGACTAAGACGCTGTCTCAAAAAAAA |
| | | ACAAAAACGACAAAAAAAAAACAAAACAGA |
| | | AAAAATAAACTAAGGCAATGACAGTCCCTG |
| | | GCAAATGCTGGGAGGGAGGCAGCAGTGGTC |
| | | AGGGAAGGTAACCCTGAAGCAGGACTTGTA |
| | | AAGCAAATAAGATTGGGAGGCCAAGGTGGG |
| | | TGGATCACGAGGTCAGGAGTTCGAGACCAG |
| | | CCTGGCCAACATAGTGAAACCCCGTCTTTA |
| | | CTAAAAATACAAAAAAATTAGCCAGGTGTG |
| | | GTGGTGGGTGCCTGTAGTCCCAGCTACTTG |
| | | GGAGGCTGAGGCAGGAGAATCTCGAACCCA |
| | | GGAGGCGGAGGTTACAGTCAGCTGAGACCG |
| | | CACCATTGCACTCCAGCCTGGGTGACAGAG |
| | | CAAGATTCCGTCTCAAAAAAAAAAAAAAAA |
| | | AAAAAACCAAGAAGAAAAGGAATGAATTAG |
| | | AACTTCTTCTGCTTGGACTTAAGGGCATCA |
| | | TCAGGCAGGTTTTGGGTAGGATAGCAGGGG |
| | | AGGCAGAGACATAGTCGGGGTCAGTGGTCA |
| | | TGAGTGTGGCTTTGAGCCCAAAAACTTGGT |
| | | TTCTGTTCCCTACTTTGCCACTCAGTAGTG |
| | | CATGACTTTGGCCAAATTTCTTAAATTCAT |
| | | GAAGCAAGTTTCCGGGTGAATGAAATGGGG |
| | | ATAAAAATAGTGTTCAAACCTATCCGTTGG |
| | | TTTGTGTGAAACTGAAATGAATAGTATCGT |
| | | GCAGGTACTTGTGAGCAAGGGGAGCTGCTG |
| | | TTTCCTGTCCCTTTATGATGGGAAATATCT |
| | | AGACAAGTTCCCAACCCTCTGCACTGCAGG |
| | | CTGCATGGCACGGAGGGTCTTGTAACACCA |
| | | GCTGGGGCTGGCCTTCTTTTAGGAGCTTCA |
| | | GTGGTTCTGAAAACTTTTATTTGTTTGTTT |
| | | GTTTTAGTAGATGTGGGGTCTTTCTGTGTT |
| | | GCCCGGACTGGTCTCAAACTTCTGGACTCA |
| | | AGTGATCCTCCCCCGCTCAACCTCCCAAAG |
| | | TGTTGGGATTACAGGTGTGAGCCACTGTGC |
| | | CCAGCCTTGAAAACTTTTTCAGGTTCTTCC |
| | | AGGGTTACTGGGCTATTAAATATTTCTATT |
| | | TCATTATAAGTCAGTTTTTCAAAGTTATAT |
| | | TATCTTAATTACCTTTTTTATATGTATTAG |
| | | TGTAGAGTAGCATTTTATATTTTGATATCC |
| | | TCCTTATGCATAGTTTTTCACTTTTTATTC |
| | | CTAGTTTTTCGTTTTTAATAAGACTTTCAA |
| | | GAAATTTATTTTATTGGCCTTTTGAAAAAA |
| | | GCAGCTTTAGATAAAGTAAGCAGTTCTGCT |
| | | TTCATTTTATAATTTATTTCTACTTTTGTT |
| | | TCATTAATCTTTTCCTCCGGCATGCCTTGG |
| | | ATTTTGTTGTGTTACTCTTTTTCTAGAGGC |
| | | TCGCATTGTGTGTCTGGTTCACTTATGATC |
| | | ACGCTTGCCTACTTTTAAGAATGGAAGAGG |
| | | GGAGGTGGAGGGTGGCTGCACAGTCGAGGG |
| | | TGTGAGGCAGTCTTGCTCTAGCCCCACCAT |
| | | GCCCTCAGCCCGCTGTGGCCACGCTGGTTC |
| | | CTCAATTGCTGGGGCGTGCAGTGTCTGTAA |
| | | GGGAGGCTACTGATGCCATCCGAGGAAGAT |
| | | GTAAGGTTTCGTGTGGGCAGCGAGAGCCTA |
| | | GCAGGCATGTGGGGTGCCCAGCAAAGGGTA |
| | | ACAGTGGACAGTTGTTGCCTCATTCCACAG |
| | | AGTTTTGATTTTTTTTTTTTTTTTAATGGT |
| | | CACTCCATCAACATCCCCCATGGCCAGAGC |
| | | CTGAGCTGGTCCCCAGAGACACAGGCATTC |
| | | AGCTGACAGCCTCGCCTTCACGCTGCTGCT |
| | | GTTCTCATGGGGGACAGGCCTCAGGTGGCA |
| | | ATGCACAAATCATTAGTTAAGGGCAGTTGT |
| | | GACAGTTACCAAGGAGTGTAGTCCCCCGCC |
| | | CCCCGCCCAGTGAAAACAGCCCTAACCAGG |
| | | GGGGGGACCTTTGGGCTCTGACCCGAAGGG |
| | | TAGGAGAAGCTGGAAGGACAGCATTCCTGT |
| | | CTGCGAAGGCAGGAGCAAAGCTGCCAGGCT |
| | | ATGAAGGAAATGGCTGGAGCCTGAAGTCAT |
| | | GCAAGCTGGGGCTGGCAGGGACAGGGCCAA |
| | | CTTCCAGGCCTGGGGGCCACCATGAGGATT |
| | | CAGGACGTGACCCCCAGGGCACATGAAGGC |
| | | CTTCCATCTGTATTTAAGAAAAGACTTTAT |
| | | CAGACGAGTATGGTGGCTCACGCCTGAATC |
| | | TTAGCACTTTGGGAGGCTGAGGCAGGTGGA |
| | | TCACGAGGTCAGGAGTTCAATACCAGCCTG |
| | | GCCAATATGGTAAAACCCCATCTCTACTAA |
| | | AACTACAAAAATTAGCCAGGCATGGTGGCG |
| | | CACGCCTGTAGTCCCAGCTACTCGGGAGGC |
| | | TGAGGCAGAAGAATCACTTGAACCCGGGAG |
| | | GTGGAGGTTACAGTGAGCCAAGATCGCGCC |
| | | ACTACACTCCAGCCTGGGTGACAGAGTGAG |
| | | ACTCCGTCTCAAAAAAACCAAAAGACTTTA |
| | | TCTTATTTCCTATATGTTTGTGGTTTCAGT |
| | | CCTGATGTATAATTTGACCCTAGTTAGAAT |
| | | GGTTATCTGAGGAAGTGGCCTGTACGATTT |
| | | CTGCTTTTTTAAATGTGTGGCTCCCTTTCT |
| | | TCATTGATTAACGTATGATTATTTTTATAA |
| | | ATGTTCCATGGCAGTGGGAAGGGATTCTCT |
| | | GTCACATTCCACATCTGGATCAGTTCCTCC |
| | | CCATTTTGTTGGTCAAATCCGATCTGCCAT |
| | | ATCCTGTGTAATGACAAGTGAGTTGCATTC |
| | | TCACCGTCACTCCTGGGGTCTCTCCGCTTC |
| | | CCCTGAGCTGGCTCAGCAGTCTGCTCCATG |
| | | TGTTTTGATGCAGGGTGACCCATTGGTATT |
| | | CCCGACACTAACGCCCCCGTCTGTGGACTG |
| | | CTTGCTGCTTGGGCTTCACTGTGTCTGGTG |
| | | TTGACAGTGCAGACCTAAAGGTGTGCACAC |
| | | ATGTGCACACACACTCCGCTGTCTTCTTGT |
| | | TTGCACTGGACTTAAATATCTATGAGGGTT |
| | | ATTTTCAACTGCTGAATTTGGAATGATTTT |
| | | TATATCTTTTCTGCTTTCTGCCCATGTACA |
| | | TGTGTTTATTTTACACTGTTGTGATTGGTA |
| | | GTTACTATGTGGGGACACAATTACTTGGGC |
| | | TGAAATAATCCACCTGTTGTGGTTGGGGTC |
| | | CTCTGGGGCATTCCAGGGTGAGAGGTTGTC |
| | | ACTGCCACCTGGGCCATGTGGGCCGGCACC |
| | | AGCATTTTGTGGTTACGAATTCTACAGTCA |
| | | CAAATATCTTTGGGCAAATCCCCTTCTATA |
| | | CCTCAAGGCAGCTTTTGGTTTGCAACCCCA |
| | | CTGGCCAGAGGGAAGGGCCAGTCACTTGGC |
| | | TCTCTCACTGCCCTGCGCCCCAGATGGTTC |
| | | TAGGGCTGCTGTTTTCCCTTGGCCCTGCCA |
| | | ACACCACTGTTTTTACTTCTGCTCATTGGC |
| | | TGAGTGCAGTGGTTCCTGGAAGCCAGTGGC |
| | | ACGTTTCCCCGCGTAGCTCGCTTATCCCAC |
| | | AGCACACACCCAAGGGTTCTGTTGCTAACA |
| | | CGCTGAATTAATTCTTTGCTCATCTTACAG |
| | | AGTGTGTTTTGACTGCCCCCATTTCTGAGG |
| | | CCTTGTAAGGCCAGAGCTTTGTTGCTTCAT |
| | | CGGCAGGTTGGGACTTAGATGGCCGTGAAT |
| | | GTTTCCTCTCTGCTGCTGCAGTAAGTAAGT |
| | | GCCCGCACCATAGTGTGTTTGGAGGCTGAA |
| | | GTTGAAGCGAGGCTGTGAGGGGAGATGGAC |
| | | GTGTGAGGAGGGATGATGGGGCTTGAGCAA |
| | | AGTGGGGGAGGGGGCAAAGGCAGTTGGCCC |
| | | AACACATTCCCCACCCCTTTGAGAGGTCTG |
| | | AGGCCTGCAGACCTGGCTCGGAGCCCACCT |
| | | GGTAGTCCTCAGACTGTGTGTGTGTGTGTG |
| | | TGTGTGTGTGTGTGTGTGTGTGTGTGTGTG |
| | | TGTGTGTGTGTGTAAAAGAGAGAAGTTGTG |
| | | GAGAAATGGGGGGCTGATTCTGCTCAGATT |
| | | CATCAGGATGAGTAGAAGGCACCCAGCTCT |
| | | CACCCTGGCCTGACATGTGTGTCCCTGAGC |
| | | AGGTTACAGTCCTCTCTGAGCCTCTGCTTC |
| | | CCATCTGGACCCTGCTGGGCAGGGCTTCTG |
| | | AGCTCCTTAGCACTAGCAGGAGGGGCTCCA |
| | | GGGGCCCTCCCTCCATGGCAGCCAGGACAG |
| | | GACTCTCAAATGAGGACAGCAGAGCTCGTG |
| | | GGGGGCTCCCACGGACCCGCCGTGGGCCCA |
| | | GGGGAGGCAGAGCCTGAGCCAACAGCAGTG |
| | | GTGCTGTGGACCGTGGATCCTGAGGGTGGC |
| | | CTGGGGCAAGTACCGGCTGAGGGTCCAGGT |
| | | GGGCTTTGTGTACCTTTGGGTCCTGGGGCC |
| | | CTGGTGACTTGGACTCCAGGTTAGAGTCAA |
| | | GTGACAGGAGAAAGGCTGGTGGGGCCCTGT |
| | | GCTTCCGACTTCATTTCGAGTGATGGCAGT |
| | | TCCCAGGAAGGAATCCACAGCTGACGGTGG |
| | | CTGACAGATCAGAGAATGGAAGGCGAGGCA |
| | | GGCGGGCGTCTGCGTGACCTCAGGTGCTTG |
| | | GGGCCCAGCAGACCCAGAGAACCATTTCCA |
| | | CTAGGCCAGGGTGCCGGAAGTGTCCACAGG |
| | | TCTTAGATTCCCTGTTCAGATGAAAAGATT |
| | | TGTGCCTTTAATGATAAAAGTGATCTGCAT |
| | | AGAGTCAAAAATTCAAGCCATGGGTATAAA |
| | | ATGCAAGTAAAATCCCTGCCCTCACCTATC |
| | | CCACCCTACTACACAGAGATGTCCTCTCGA |
| | | GTTTCCTAGACTCACTCTGGAAATTTCTGT |
| | | ATACACACAGAAGCTTGTGCCTCTGCTCGT |
| | | GAAGGCAGAGGGAGGGAGAGCTGAAGGGCC |
| | | AGCACCTTCTCACCTGTGGGCCCCCTCAGT |
| | | GCTCGGTCCCAGAGCATGCAGGACTGTGCC |
| | | TCGTGTTCAGTTTGCTGGTCTGACTTCATG |
| | | CTCCTTGGGCAGGATATGCATGTGCCATGC |
| | | TAGGAGACATGTGGATGTGAAGCTGGGGGA |
| | | CAATGTCCCCTGGCTATGCCTTTACAAGGG |
| | | AAGTAAGGAAGGTAGGAGGTGAGCCTGGGA |
| | | GGGAGGGAGGGAGGCGCGGAGCCGCCGCAG |
| | | GTGTTTCTTTTACTGAGTGCAGCCCATGGC |
| | | CGCACTCAGGTTTTGCTTTTCACCTTCCCA |
| | | TCTGTGAAAGAGTGAGCAGGAAAAAGCAAA |
| | | A |
| 2 | HNRNPH1 | CATTTCGTCTTAGCCACGCAGAAGTCGCGT |
| | (e.g. NCBI | GTCTAGTTTGTTTCGACGCCGGACCGCGTA |
| | Reference | AGAGACGATGATGTTGGGCACGGAAGGTGG |
| | Sequence: | AGAGGGATTCGTGGTGAAGGTCCGGGGCTT |
| | NM_ | GCCCTGGTCTTGCTCGGCCGATGAAGTGCA |
| | 005520.3) | GAGGTTTTTTTCTGACTGCAAAATTCAAAA |
| | | TGGGGCTCAAGGTATTCGTTTCATCTACAC |
| | | CAGAGAAGGCAGACCAAGTGGCGAGGCTTT |
| | | TGTTGAACTTGAATCAGAAGATGAAGTCAA |
| | | ATTGGCCCTGAAAAAAGACAGAGAAACTAT |
| | | GGGACACAGATATGTTGAAGTATTCAAGTC |
| | | AAACAACGTTGAAATGGATTGGGTGTTGAA |
| | | GCATACTGGTCCAAATAGTCCTGACACGGC |
| | | CAATGATGGCTTTGTACGGCTTAGAGGACT |
| | | TCCCTTTGGATGTAGCAAGGAAGAAATTGT |
| | | TCAGTTCTTCTCAGGGTTGGAAATCGTGCC |
| | | AAATGGGATAACATTGCCGGTGGACTTCCA |
| | | GGGGAGGAGTACGGGGGAGGCCTTCGTGCA |
| | | GTTTGCTTCACAGGAAATAGCTGAAAAGGC |
| | | TCTAAAGAAACACAAGGAAAGAATAGGGCA |
| | | CAGGTATATTGAAATCTTTAAGAGCAGTAG |
| | | AGCTGAAGTTAGAACTCATTATGATCCACC |
| | | ACGAAAGCTTATGGCCATGCAGCGGCCAGG |
| | | TCCTTATGACAGACCTGGGGCTGGTAGAGG |
| | | GTATAACAGCATTGGCAGAGGAGCTGGCTT |
| | | TGAGAGGATGAGGCGTGGTGCTTATGGTGG |
| | | AGGCTATGGAGGCTATGATGATTACAATGG |
| | | CTATAATGATGGCTATGGATTTGGGTCAGA |
| | | TAGATTTGGAAGAGACCTCAATTACTGTTT |
| | | TTCAGGAATGTCTGATCACAGATACGGGGA |
| | | TGGTGGCTCTACTTTCCAGAGCACAACAGG |
| | | ACACTGTGTACACATGCGGGGATTACCTTA |
| | | CAGAGCTACTGAGAATGACATTTATAATTT |
| | | TTTTTCACCGCTCAACCCTGTGAGAGTACA |
| | | CATTGAAATTGGTCCTGATGGCAGAGTAAC |
| | | TGGTGAAGCAGATGTCGAGTTCGCAACTCA |
| | | TGAAGATGCTGTGGCAGCTATGTCAAAAGA |
| | | CAAAGCAAATATGCAACACAGATATGTAGA |
| | | ACTCTTCTTGAATTCTACAGCAGGAGCAAG |
| | | CGGTGGTGCTTACGAACACAGATATGTAGA |
| | | ACTCTTCTTGAATTCTACAGCAGGAGCAAG |
| | | CGGTGGTGCTTATGGTAGCCAAATGATGGG |
| | | AGGCATGGGCTTGTCAAACCAGTCCAGCTA |
| | | CGGGGGCCCAGCCAGCCAGCAGCTGAGTGG |
| | | GGGTTACGGAGGCGGCTACGGTGGCCAGAG |
| | | CAGCATGAGTGGATACGACCAAGTTTTACA |
| | | GGAAAACTCCAGTGATTTTCAATCAAACAT |
| | | TGCATAGGTAACCAAGGAGCAGTGAACAGC |
| | | AGCTACTACAGTAGTGGAAGCCGTGCATCT |
| | | ATGGGCGTGAACGGAATGGGAGGGTTGTCT |
| | | AGCATGTCCAGTATGAGTGGTGGATGGGGA |
| | | ATGTAATTGATCGATCCTGATCACTGACTC |
| | | TTGGTCAACCTTTTTTTTTTTTTTTTTTTT |
| | | TTCTTTAAGAAAACTTCAGTTTAACAGTTT |
| | | CTGCAATACAAGCTTGTGATTTATGCTTAC |
| | | TCTAAGTGGAAATCAGGATTGTTATGAAGA |
| | | CTTAAGGCCCAGTATTTTTGAATACAATAC |
| | | TCATCTAGGATGTAACAGTGAAGCTGAGTA |
| | | AACTATAACTGTTAAACTTAAGTTCCAGCT |
| | | TTTCTCAAGTTAGTTATAGGATGTACTTAA |
| | | GCAGTAAGCGTATTTAGGTAAAAGCAGTTG |
| | | AATTATGTTAAATGTTGCCCTTTGCCACGT |
| | | TAAATTGAACACTGTTTTGGATGCATGTTG |
| | | AAAGACATGCTTTTATTTTTTTGTAAAACA |
| | | ATATAGGAGCTGTGTCTACTATTAAAAGTG |
| | | AAACATTTTGGCATGTTTGTTAATTCTAGT |
| | | TTCATTTAATAACCTGTAAGGCACGTAAGT |
| | | TTAAGCTTTTTTTTTTTTTAAGTTAATGGG |
| | | AAAAATTTGAGACGCAATACCAATACTTAG |
| | | GATTTTGGTCTTGGTGTTTGTATGAAATTC |
| | | TGAGGCCTTGATTTAAATCTTTCATTGTAT |
| | | TGTGATTTCCTTTTAGGTATATTGCGCTAA |
| | | GTGAAACTTGTCAAATAAATCCTCCTTTTA |
| | | AAAACTGCA |

TABLE 2
Primer Sequences for experiments.

| SEQ | | |
|---|---|---|
| ID | Real-Time qPCR | |
| NO: | primers | SEQUENCE (5′ -> 3′) |
| 3 | 18S Forward | AACTTTCGATGGTAGTCGCCG |
| 4 | 18S Reverse | CCTTGGATGTGGTAGCCGTTT |
| 5 | INCR1 Forward | GTGGGATATGACAGGGACGC |
| 6 | INCRI Reverse | GGCAGGAGCAGCTAATCCAA |
| 7 | PD-L1 Forward | GAGTGGTAAGACCACCACC |
| 8 | PD-L1 Reverse | GGTTTTCCTCAGGATCTAAT |
| 9 | PD-L2 Forward | AGTGCTATCTGAACCTGTGGTC |
| 10 | PD-L2 Reverse | AGTGCTGGGTCATCCAAAGG |
| 11 | RICI Forward | CTCCGCACGGACCATGTATT |
| 12 | RICI Reverse | TCGGACTGAACGTGGAAAGG |
| 13 | JAK2 Forward | TGGGGTTTTCTGGTGCCTTT |
| 14 | JAK2 Reverse | TAGAGGGTCATACCGGCACA |
| 15 | STATI Forward | GTTATGGGACCGCACCTTCA |
| 16 | STATI Reverse | CAGTGAACTGGACCCCTGTC |
| 17 | IDO1 Forward | TCTGGCCAGCTTCGAGAAAG |
| 18 | IDO1 Reverse | AGAACTAGACGTGCAAGGCG |
| 19 | HNRNPH1_Forward | CCAGAGCAGCATGAGTGGAT |
| 20 | HNRNPH1 Reverse | TAGCTGCTGTTCACTGCTCC |
| 21 | MALATI Forward | AGCAGACACACGTATGCGAA |
| 22 | MALATI Reverse | GTGGTTCCCAATCCCCACAT |
| 23 | NORAD Forward | GTCCTGACGACAACGGACAA |
| 24 | NORAD Reverse | AGAATGAAGACCAACCGCCC |
| 25 | RMRP Forward | CACGTAGACATTCCCCGCTT |
| 26 | RMRP Reverse | CTGCCTGCGTAACTAGAGGG |
| 27 | GAPDH Forward | CCAGCAAGAGCACAAGAGGA |
| 28 | GAPDH Reverse | ACATGGCAACTGTGAGGAGG |
| 29 | NFKB1 Forward | GTGAAGACCACCTCTCAGGC |
| 30 | NFKB1 Reverse | CTGTCGCAGACACTGTCACT |
| 31 | NFKB2 Forward | GCGTTGTCAACCTCACCAAC |
| 32 | NFKB2 Reverse | GAGTCTCCATGCCGATCCAG |
| 33 | RELB Forward | CATGGCATCGAGAGCAAACG |
| 34 | RELB Reverse | GACACGGTGCCAGAGAAGAA |
| 35 | CSF2 Forward | TGATGGCCAGCCACTACAAG |
| 36 | CSF2 Reverse | CCAGCAGTCAAAGGGGATGA |
| | Primers for | |
| | rapid | |
| SEQ | amplification | |
| ID | of cDNA | |
| NO: | ends | SEQUENCE (5′ -> 3′) |
| 37 | QT Primer | CCAGTGAGCAGAGTGACGAGGACTCGAGCTCAA |
| | | GCTTTTTTTTTTTTTTTTT |
| 38 | QI Primer | GAGGACTCGAGCTCAAGC |
| 39 | QO Primer | CCAGTGAGCAGAGTGACG |
| 40 | GSP-RT | CAGTCAGGAAGAAGGCAGCA |
| 41 | 5′GSP1 | GTGTGTAGGGAAGGGACAGC |
| 42 | 5′GSP2 | CCGAATTGGCCGCAGGTACC |
| 43 | 3′GSP1 | TAATACGACTCACTATAGGCTGAATATATTTATT |
| | | TGCATTTG |
| 44 | 3′GSP2 | TAATACGACTCACTATAGGAAAAAGTAAAGACT |
| | | AGAAATTCTTTTTG |
| SEQ | Primers for | |
| ID | INCR1 full- | |
| NO: | lengh amplification | SEQUENCE (5′ -> 3′) |
| 45 | INCR1 Full- | CAGCAGCGACTTCATCCAC |
| | length Forward | |
| 46 | INCR1 Full- | TAGATGAGAGGCCTGATCTTTA |
| | length_Reverse | |
| SEQ | Primers for INCR1 mini- | |
| ID | gene generation and | |
| NO: | RNA pull-down assay | SEQUENCE (5′ -> 3′) |
| 47 | INCR1 Exonl Forward | GTGCCTGCAGCCGGCGGG |
| 48 | INCR1 Intron 1 Reverse | cttgcggccgctaaccgTCTTTCTTCTATTTCAGACTCCTC |
| | | C |
| 49 | INCR1 Intron 1 Forward | cggttagcggccgcaagCAATGAAGCATGCAGGTCTGGG |
| 50 | INCR1 Exon 2 Reverse | CTTGTCATTTCATGTCTCTTTAATTAAAGAG |
| 51 | pCR2.1 - HindIII Reverse | ggttaagcttAGATGCATGCTCGAGCGG |
| 52 | Frag 1 Forward T7 | taatacgactcactatagGGTGGGTCTGGCACGAAGG |
| 53 | Frag 1 Reverse | TGTGACGGACGCAACTGG |
| 54 | Frag 2 Forward T7 | taatacgactcactataggAATACATGGTCCGTGCGG |
| 55 | Frag 2 Reverse | CGCGGCCTGAGGAAGGGG |
| 56 | Frag 3 Forward T7 | taatacgactcactatagGCGGGAGGAGCGCGGAGGC |
| 57 | Frag 3 Reverse | CCCTAGACTCGCCCCACTTG |
| 58 | Frag 4 Forward T7 | taatacgactcactatagggGACCTAGGCAACGGCCT |
| 59 | Frag 4 Reverse | ACCGGCAGGAGCTTCATCAC |
| 60 | Frag 5 Forward T7 | taatacgactcactataggCTTTAATGCCGGGGTCTG |
| 61 | Frag 5 Reverse | AAGCTAAATATGGACCTACGCT |
| 62 | Frag 6 Forward T7 | taatacgactcactataggAGTTTTAGAACTGGAAGGG |
| 63 | Frag 6 Reverse | TCTTTCTTCTATTTCAGACTCCTCC |
| 64 | Frag 7 Forward T7 | taatacgactcactataggCAATGAAGCATGCAGGTCTGGG |
| 65 | Frag 7 Reverse | CTGCAAAAGAAAACAACAAAAAAACTAAAG |
| SEQ | | |
| ID | Antisense | SEQUENCE (5′ -> 3′) |
| NO: | oligonucleotides (ASOs) | |
| 66 | LNA GapmeR Negative | AACACGTCTATACGC |
| | Control | |
| 67 | LNA GapmeR INCR1 | TTACATGATGACCTTT |
| 68 | ASO Negative Control | GCGACTATACGCGCAATATG |
| 69 | ASO HIB | CTCCAGCTCCCCCCGGCAAC |
| SEQ | Probes for RNA | |
| ID | antisense purification | |
| NO: | assay | SEQUENCE (5′ -> 3′) |
| 70 | INCRI probel | /5Biosg/ATTATTATAGATGAGAGGCCTGATCTTTA |
| | | TTGAAAACATATTCCAAGTGTTGAAGACTTTTCA |
| | | TTCTTGTAAGTCCATACTTATTTTCAAA |
| 71 | INCR1 probe2 | /5Biosg/CAGAACAGCATAGTCTGTTCATTCATTCA |
| | | TTCAATTCATGAATTCATTCACATAATTATCCAA |
| | | TTTCTTGAGCACCTATTTGATAGTCACTG |
| 72 | INCR1 probe3 | /5Biosg/TTACTTTTTTATTACAAATGGAATACGTA |
| | | TACTTGCAAATAATTCAGATACTGTGGAAGAGAT |
| | | CAAATGAATTGCAAAAGTGTCCCTCCTC |
| 73 | INCR1 probe4 | /5Biosg/CCTTCACCACTATCTCCCATGGCATGCAG |
| | | AGAGAGTAACCATTATTTGTGTGTCCCTCCAGAA |
| | | ATTTTTTTATTCAACTACTATTTTTTTAT |
| 74 | INCR1 probe5 | /5Biosg/TAAAAAAGACTTGAAGTCACCCTATGAA |
| | | GACAAAAAATAATCACATTAAGTGTGAAAGAAC |
| | | CTATTCTTCCAGTACAGGATAAGCCATACT |
| 75 | INCRI probe6 | /5Biosg/CACAGTTATGTTATCAGGTCACTTGAGTT |
| | | CAAAGTTTTGTGTTGGCACTAGCTAAGTAAAGGA |
| | | AAACACCTCTGCTTTCATTGTTGAGTTTC |
| 76 | INCRI probe7 | /5Biosg/TATTTCCTGCAATGGCGTCCCTGTCATAT |
| | | CCCACAATGGCCTCCCTGCCATTTGGATATCCCT |
| | | TCCATATCCTGTTGAAATTACTCCCTAATA |
| 77 | INCR1 probe8 | /5Biosg/CCTTGGTCTTTCCATTCCTGGGCTAACTA |
| | | CCATCAATCTGAGGGCTAACAATACAAGTAGAA |
| | | AAAGTATACATTTGTCACTGATCACTGATC |
| 78 | INCRI probe9 | /5Biosg/TGGTCCCTGATACTGGCTCAGGTAATGCC |
| | | ACTATTGTCAAGAAGATACCACTTGTAAAGTAG |
| | | ATTTAATTTTCATTATATTTTACCATATGC |
| 79 | INCR1 probe 10 | /5Biosg/AAGATATTAAAGAAATCATCATGACTTA |
| | | GCCTCATCAACAGCATTGCTAGATCTGGGATGGA |
| | | AAGGAAGAGTATAATCCTGGCAGTCAGGA |
| 80 | INCR1 probe11 | /5Biosg/AGTAGGGAAGGGTTTTATTCAGGTCGTCT |
| | | GGGCTCCATAATATCCCTTGTGTATCTGCAGTCT |
| | | CCTTTGCCATGGATCAACACAATAGGAAA |
| 81 | INCR1 probe 12 | /5Biosg/TGTCTCTTTAATTAAAGAGTTTTGATTTCA |
| | | GAGGAGGGTACCTGCCTTCGCAGTCCGCGTCCTC |
| | | GTTCCACCGAATTGGCCGCAGGTACCCG |
| 82 | INCR1 probe13 | /5Biosg/CCGGCTGCAGGCACGTCCTCCAGCCGGC |
| | | GGTCCGGGGTGGATGAAGTCGCTGCTGCCGAAA |
| | | ATCCCTGAGCCTCCCCGCGTTGGACCGACGGC |
| 83 | INCR1 probe 14 | /5Biosg/CGCGGCGTGCAGACTCCGCACGCTCCCCG |
| | | GCCGAGCCCAGACTTGTTCTTCTCTCCCTTCCCCC |
| | | ATTCCCCGAGGGACAAATTTGTTTAAACT |
| 84 | Scrambled probe | /5Biosg/AGGAACGCCACTCTATCCGACCACGGAT |
| | | CCTCGTCCATTCCGACACTAGGGTCTCAACCGGT |
| | | AAGTCCTGGTTGCGTGTTCATCTACGACCGA |
| SEQ | | |
| ID | | |
| NO: | sgRNAs for CRISPRi | SEQUENCE (5′ -> 3′) |
| 85 | sgRNA INCRI oligo1 | CACCGTACTTTAAAGGTCGCCGTTT |
| 86 | sgRNA INCRI oligo2 | AAACAAACGGCGACCTTTAAAGTAC |
| | RNA oligo- | |
| SEQ | nucleotides for | |
| ID | RNA EMSA and Micro- | |
| NO: | scale thermophoresis | SEQUENCE (5′ -> 3′) |
| 87 | INCR1 | GGGGCGGGCCGGUUGCCGGGGGGAGCUGGAGU |
| | | CACGUGACGGC/36-FAM/ |
| 88 | PD-L1 | UAAGGGGUGAGGGGCAGGAGAAUGGGUAUGGA |
| | | UGGAGGUAGA/36-FAM/ |
| 89 | JAK2 | AUACGUGGGAGUGGGGUGCUGAGCUGGGCAGG |
| | | GUGAGAGUAU/36-FAM/ |
| | Primers for determining | |
| | HNRNPH1 splice | |
| | variants | SEQUENCE (5′ -> 3′) |
| 90 | PDL1 E5E6 For set3 | TCTGGGAGCCATCTTATTATGC |
| | (spliced) | |
| 91 | PDL1 E5E6 Rev set3 | GTTTGTATCTTGGATGCCACATT |
| | (spliced) | |
| 92 | PDL1 E515 For set3 | GGTAATTCTGGGAGCCATCTT |
| | (unspliced) | |
| 93 | PDL1 E515 Rev set3 | CTTGCAGCCATGATCCAATTC |
| | (unspliced) | |

### REFERENCES

1. Pardoll D M. The blockade of immune checkpoints in cancer immunotherapy. Nat Rev Cancer. 2012; 12(4):252-64. Epub 2012/03/23.
2. Sharma P, Allison J P. The future of immune checkpoint therapy. Science. 2015; 348(6230):56-61. Epub 2015/04/04.
3. Beatty G L, Gladney W L. Immune escape mechanisms as a guide for cancer immunotherapy. Clin Cancer Res. 2015; 21(4):687-92. Epub 2014/12/17.
4. Garcia-Diaz A et al., Interferon Receptor Signaling Pathways Regulating PD-L1 and PD-L2 Expression. Cell Rep. 2017; 19(6):1189-201. Epub 2017/05/13.
5. Freeman G J, et al., Engagement of the PD-1 immunoinhibitory receptor by a novel B7 family member leads to negative regulation of lymphocyte activation. J Exp Med. 2000; 192(7):1027-34. Epub 2000/10/04.
6. Antonia S J, et al., Durvalumab after Chemoradiotherapy in Stage III Non-Small-Cell Lung Cancer. N Engl J Med. 2017; 377(20):1919-29. Epub 2017/09/09.
7. Larkin J, et al., Combined Nivolumab and Ipilimumab or Monotherapy in Untreated Melanoma. N Engl J Med. 2015; 373(1):23-34. Epub 2015/06/02.
8. Topalian S L, et al., Safety, activity, and immune correlates of anti-PD-1 antibody in cancer. N Engl J Med. 2012; 366(26):2443-54. Epub 2012/06/05.
9. Geisler S, Coller J. RNA in unexpected places: long non-coding RNA functions in diverse cellular contexts. Nat Rev Mol Cell Biol. 2013; 14(11):699-712. Epub 2013/10/10.
10. Prensner J R, Chinnaiyan A M. The emergence of lncRNAs in cancer biology. Cancer Discov. 2011; 1(5):391-407. Epub 2011/11/19.
11. Atianand M K, Fitzgerald K A. Long non-coding RNAs and control of gene expression in the immune system. Trends in molecular medicine. 2014; 20(11):623-31. Epub 2014/09/30.
12. Mineo M, et al., The Long Non-coding RNA HIF1A-AS2 Facilitates the Maintenance of Mesenchymal Glioblastoma Stem-like Cells in Hypoxic Niches. Cell Rep. 2016; 15(11):2500-9. Epub 2016/06/07.
13. Gutschner T, et al., The noncoding RNA MALAT1 is a critical regulator of the metastasis phenotype of lung cancer cells. Cancer Res. 2013; 73(3):1180-9. Epub 2012/12/18.
14. Huarte M, et al., A large intergenic noncoding RNA induced by p53 mediates global gene repression in the p53 response. Cell. 2010; 142(3):409-19. Epub 2010/08/03.
15. Tseng Y Y, et al., PVT1 dependence in cancer with MYC copy-number increase. Nature. 2014; 512(7512):82-6. Epub 2014/07/22.
16. Atianand M K, et al., A Long Noncoding RNA lincRNA-EPS Acts as a Transcriptional Brake to Restrain Inflammation. Cell. 2016; 165(7):1672-85. Epub 2016/06/18.
17. Carpenter S, et al., A long noncoding RNA mediates both activation and repression of immune response genes. Science. 2013; 341(6147):789-92. Epub 2013/08/03.
18. Mineo M, et al., Tumor Interferon Signaling Is Regulated by a lncRNA INCR1 Transcribed from the PD-L1 Locus. Mol Cell. 2020; 78(6):1207-23 e8. Epub 2020/06/07.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of selecting a treatment for, and optionally treating, a subject who has a tumor, the method comprising:
obtaining a sample comprising a plurality of cells from the tumor of the subject;
determining a tumor expression level of nuclear paraspeckle assembly transcript 1 (NEAT1) and/or Heterogeneous Nuclear Ribonucleoprotein H1 (HNRNPH1) in the sample;
comparing the tumor expression level of NEAT1 and/or HNRNPH1 to a reference expression level of NEAT1 and/or HNRNPH1;
selecting an immunotherapy treatment for a subject who has a level of NEAT1 above the reference level and/or a level of HNRNPH1 below the reference level; and
administering the immunotherapy treatment to the subject.

2. A method for determining if a subject who has a tumor is likely to benefit from an immunotherapy treatment, the method comprising:
obtaining a sample comprising a plurality of cells from the tumor of the subject;
determining an expression level of NEAT1 and/or HNRNPH1 in the sample; and
comparing the tumor expression levels of NEAT1 and/or HNRNPH1 to a reference expression level of the NEAT1 and/or HNRNPH1,
wherein the tumor expression level of NEAT1 and/or HNRNPH1 in comparison to the reference expression level of NEAT1 and/or HNRNPH1 indicates whether the subject is likely to benefit from treatment with immunotherapy.

3. The methods of claim 1, wherein an expression level of NEAT1 in the sample that is above the reference expression level of NEAT1 indicates a likelihood of response to the immunotherapy treatment.

4. The method of claim 1, wherein an expression level of HNRNPH1 that is below the reference expression level of HNRNPH1 indicates a likelihood of response to the immunotherapy treatment.

5. The method of claim 1, wherein the sample is a fresh tumor sample.

6. The method of claim 1, wherein the sample is a fixed tumor sample.

7. The method of claim 1, wherein the immunotherapy treatment comprises administration of an immune checkpoint inhibitor.

8. The method of claim 7, wherein the immune checkpoint inhibitor is selected from an inhibitor of PD-1, an inhibitor of PD-L1, an inhibitor of CTLA-4, an inhibitor of Lag3, an inhibitor of CD40, an inhibitor of CD137, an inhibitor of OX40, and an inhibitor of Tim3.

9. The method of claim 8, wherein the immune checkpoint inhibitor is an anti-PD-1 antibody or anti-PD-L1 antibody.

10. The method of claim 1, wherein the method further comprises administering an additional treatment.

11. The method of claim 10, wherein the additional treatment is selected from a second immune checkpoint inhibitor, a resection, a chemotherapy, and radiation.

12. The method of claim 11, wherein the second immune checkpoint inhibitor is not an inhibitor of PD-1 or is not an inhibitor of PD-L1.

13. The method of claim 11, wherein the second immune checkpoint inhibitor is an inhibitor or CTLA-4, an inhibitor of Lag3, or an inhibitor of Tim3.

14. The method of claim 12, wherein the method further comprises administering a second additional treatment.

15. The method of claim 14, wherein the second additional treatment is CAR T therapy.

16. The method of claim 1, wherein the sample is from a glioblastoma cancer tumor or a carcinoma cancer tumor.

17. The method of claim 16, wherein the carcinoma cancer tumor is a melanoma cancer tumor.

18. The method of claim 1, wherein the subject is a mammal.

19. The method of claim 18, wherein the mammal is a human or a non-human veterinary subject.

20. The method of claim 13, wherein the method further comprises administering a second additional treatment.
